SEQUENCE LISTING (3 sequences)

SEQ ID NO: 1 - DNA, 671 bp, Enterobacter sp.
cacatcgcatacgtcgcagaccaaagtgggggaccttcgggcctcatgccatcagatgtgcccagatgggattagctagtaggtggggtaatggctcacctaggcgacgatccctagctggtctgagaggatgaccagccacactggaactgatacacggtccagactcctacgggaggcagcagtggggaatattgatttatgggcgcaagcctgatgcagccatgccgcgtgtatgaacaaggccttccgattgtaaattgctttctccgaataggaaggcctgctggttaataaccttgcggattgactttactcgcaaacgaagcaccggctaactccgtgccttaagcccttcctcctcggagggtgcactttttaatccgaattactggttcttaagcgcacgctggctgcctgtcgcttgcgatgtgaaatccccgggctccacctgggaactgcattcgaaactggaccgctagagtcttgtagaggggggtggaattcctcgtgtaccggtgaaatgcgtacagatctggaagaataccccccaccaaggcggccccctggacaaagactgactctcaggtgcaaaaccgtggggagcccacttgattatataccctggtagtccactccgctaccgatgtcaacttgattcccccctccaa

SEQ ID NO: 2 - DNA, 20 bp, Artificial Sequence
  PCR Primer
tggagggggataactactgg

SEQ ID NO: 3 - DNA, 18 bp, Artificial Sequence
  PCR Primer
ggcacaacctccaagtcg

(19) United States
(12) Patent Application Publication
(10) Pub. No.: US 2009/0137013 A1
(43) Pub. Date: May 28, 2009

(54) MICROORGANISMS AND METHODS FOR INCREASED HYDROGEN PRODUCTION USING DIVERSE CARBONACEOUS FEEDSTOCK AND HIGHLY ABSORPTIVE MATERIALS

(75) Inventors: Elmar Schmid, Escondido, CA (US); James Gibson, Escondido, CA (US)

Correspondence Address: Patentique PLLC, P.O. Box 5803, Bellevue, WA, 98006, US

(73) Assignee: Sustainable Green Technologies, Inc., Escondido, CA (US)

(21) Appl. No.: 12/267,529

(22) Filed: Nov. 7, 2008

Related U.S. Application Data
(63) Continuation-in-part of application No. 12/132,574, filed on Jun. 3, 2008, now abandoned.
(60) Provisional application No. 61/002,435, filed on Nov. 7, 2007.
(60) Provisional application No. 61/083,374, filed on Jul. 24, 2008.

(51) Int. Cl. C12P 7/64; C12P 3/00; C12P 19/04; C12P 7/62; C12P 7/16; C12N 1/20

(52) U.S. Cl. 435/134; 435/168; 435/135; 435/158; 435/160; 435/252.1; 435/252.7

(57) ABSTRACT

The disclosed invention relates to an isolated hydrogen gas producing microorganism, termed Enterobacter sp. SGT-T4™ and derivatives thereof. Compositions and methods comprising the disclosed microorganisms are also provided. The disclosed invention also relates to a method to increase the hydrogen production rate and yield of hydrogen gas producing microorganism in the presence of diatomaceous earth and other absorptive materials. Further, the disclosure relates to the production of high microalgal biomass and microalgal oils suitable for economical industrial scale bio-diesel production from processed bacterial fermentation wastes as feedstock using the green microalga Chlorella protothecoides.

### RELATED APPLICATIONS

This application claims benefit of priority from U.S. Provisional Patent Application No. 61/002,435, filed Nov. 7, 2007 and entitled “Method for increased microbial hydrogen production using diatomaceous earth and other highly absorptive materials”; U.S. Non-Provisional patent application Ser. No. 12/132,574, filed Jun. 3, 2008 and entitled “Hydrogen producing microorganism useful for energy generation from diverse carbonaceous feedstock”; and U.S. Provisional Patent Application No. 61/083,374, filed Jul. 24, 2008 and entitled “Method for microbial oil production from microbial fermentation waste using the green microalgae Chlorella protothecoides”. All three of these applications are incorporated herein in their entireties as if fully each was fully set forth.

### FIELD OF THE DISCLOSURE

The disclosure relates to the field of hydrogen gas production, and increasing the effectiveness of hydrogen generating microorganisms. The disclosure includes a process and conditions to increase the hydrogen production rate of hydrogen producing microorganisms, such as the Enterobacter sp. SGT 06-1™, using diatomaceous earth (DE), silicates, cellulose, metal oxides and other absorptive materials. The disclosed process describes a method of increasing hydrogen gas production from microorganisms in the presence of defined amounts of absorptive materials, such as diatomaceous earth, zeolite or activated carbon. The process and described conditions not only allow cost-effective industrial scale generation of hydrogen energy, e.g. in the form of electricity and heat, but also the cost effective harvesting of marketable microbial metabolic end products in a bio-refinery setting.

This disclosure further relates to hydrogen gas producing microorganisms. These organisms provide an environmentally friendly and sustainable form of hydrogen-based energy production that is just starting to benefit human society. The disclosure includes an isolated and genetically unique microorganism, termed Enterobacter sp. SGT-T4™ which may be cultured in a disclosed method of the disclosure. The microorganism is metabolically versatile and generates high amounts of hydrogen gas from different renewable feedstock, including cellulosics- and hemicellulosics-derived sugars, alcoholic sugars, glycerol and glycerol-containing wastes as carbonaceous feedstock. The high hydrogen production rate of the disclosed microorganism with different feedstock is further increased in the presence of metallosilicates, such as natural zeolite. The disclosed microorganism implemented into a suitable bio-reactor environment will allow economical generation and on-site utilization of bio-hydrogen energy. This bio-hydrogen will be converted to electricity and heat by suitable means, such as a fuel cell. Sites with traditionally high amounts of cellulosics, hemicellulosics, starch, glycerol and other renewable bio-waste materials will have the ability to produce large amounts of bio-hydrogen.

The disclosure further relates to the field of economical and sustainable bio-fuel production utilizing photosynthesis-performing life forms, such as green algae and cyanobacteria. More specifically, it relates to the field of microalgal oil production from fermentation waste streams for economical and sustainable industrial scale bio-diesel production. Also disclosed are methods for the use of processed bacterial fermentation waste streams as low cost carbon- and nitrogen-containing feedstock to achieve high microalgal biomass and oil production.

### BACKGROUND OF THE DISCLOSURE

Due to the eminent danger of global warming caused by rising anthropogenic discharge of fossil fuel-derived green house gases, most prominently carbon dioxide (CO₂), into earth's atmosphere and due to escalating costs for non-renewable fossil fuels, most namely petroleum and natural gas, there is an urgent need to find ecologically more friendly fuels. Biofuels, such as bio-ethanol from corn (maize) or sugarcane and bio-diesel produced from diverse plant oils, e.g. rapeseed or palm oil, have been increasingly heralded as attractive sustainable alternatives to the currently used fossil fuels, such as petroleum, coal and natural gas. Large investments have been put into bio-ethanol and bio-diesel plants in the past years. Another alternative and attractive fuel candidate with much less media attention is hydrogen gas (H₂). Hydrogen is the single most abundant chemical element in the universe and huge amounts of hydrogen atoms are conserved in the chemical bonds of renewable biomass, such as green plants-derived sucrose, cellulose, hemicellulose, starch, lipids and fats. Combustion of hydrogen gas results in the formation of water with no emission of the green house gas carbon dioxide (CO₂) as opposed to burning fossil fuels, bio-diesel and bio-ethanol. Moreover, hydrogen gas can be directly converted to electricity with high conversion efficiencies using fuel cell technologies. Generation of hydrogen gas from suitable high hydrogenated materials can be achieved by different means, including electrochemical, steam reforming, or with biological organisms. Electrochemical generation of hydrogen gas from water requires high energy inputs to achieve the necessary hydrolysis. Industrial scale hydrogen gas production from fossil fuels by steam reforming or coal gasification bears the disadvantage that this process is accompanied with high emissions of the green house gases (GHG) carbon dioxide (CO₂) and nitrogen oxides (NOₓ) as well as of the highly poisonous carbon monoxide (CO). Therefore there is a high interest in developing economical hydrogen gas-generating technologies which are ecologically more advantageous. These technologies also show real benefits for the mandated global carbon dioxide abatement. Hydrogen energy concepts and technologies have to be developed which allow cost-effective sequestration of CO₂, and which allow hydrogen gas generation from renewable resources, e.g. plant biomass, to assure a closed carbon cycle.

Several alternative hydrogen energy generation concepts and technologies have been suggested in the past and especially the idea to produce hydrogen gas with the help of microorganisms, such as bacteria and algae, via fermentation processes has re-gained tremendous interest in the past years. Biological hydrogen can be produced both by photosynthetic and fermentative microorganisms. However, none of the described microorganisms and proposed concepts led to a proven and long-term functioning industrial scale bio-hydrogen production system. One of the main reasons which kept the studied microorganisms, including Rhodobacter sp. bacteria, the cyanobacterium Oscillatoria sp., the green algae Chlamydomonas reinhardtii, Clostridia species, Thermotogae, and diverse enterobacterial genera, from successfully and competitively entering the hydrogen energy market is their notoriously low hydrogen production rates. No process or technique has been described which allows utilization of a suitable hydrogen gas generating candidate microorganism for long term and low cost production of hydrogen gas.

Hydrogen gas generation in photosynthetic microorganisms, such as green algae or cyanobacteria, is very low primarily due to the photosynthesis-generated oxygen gas which inhibits the H₂-generating hydrogenase enzyme and also chemically reacts with the generated hydrogen gas. Photosynthetic hydrogen production further requires rather expensive incubation vessels with large, light-exposed surface areas and complex, alternating light-dark cultivation conditions. Heterotrophic microbial hydrogen producers have the advantage to be independent of solar energy input and do not require elaborate fermentation vessels for hydrogen production. However, even though heterotrophic hydrogen producers generally have much higher hydrogen production rates than photosynthetic hydrogen producing microbes, they are dependent on a suitably cheap feedstock to assure low cost hydrogen production.

Therefore, a facultative anaerobic and robust microorganism with high tolerance for oxygen levels and high hydrogen production from cheap feedstock would be advantageous for an industrial-scale biohydrogen production system. Furthermore, even though a series of mesophilic and moderate thermophilic microorganisms have been studied intensively for quantitative bio hydrogen production from common feedstock such as glucose, sucrose and maltose, no reports exist for more versatile bacteria capable of generating high amounts of hydrogen gas from other renewable biomass-derived feedstock, such as sucrose, maltose, xylose, arabinose, galactose, mannitol, sorbitol and glycerol.

Plant-derived cellulose and hemicellulose-containing materials (often referred to as cellulosics and hemicellulosics respectively) are the single most abundant renewable carbon source on earth and are annually produced by photosynthetic organism, such as grasses, shrubs and trees, on a Giga ton scale. Globally green plants convert about 190 Giga tons of carbon dioxide annually into renewable biomass mostly in the form of leaves, stems, wood, tubers and fruits. Industry-processed cellulosics, such as paper, newsprints, card board, and shopping bags, make up more than 40% of all municipal solid waste, a waste stream that to the vast extent ends up in land fills. Moreover, plant-derived oils serve as raw materials for the rapidly growing bio-diesel fuel industry which uses these renewable molecules to synthesize its biofuel using chemical methods. 3.8 million tons of bio-diesel was produced in 2005 via transesterification of oils that were extracted from a huge variety of sources including canola (rapeseed), corn, palm oil, and olives. Since glycerol is—together with salts and methanol—one of the major waste products generated during transesterification, it has in recent years flooded the glycerol market in the form of bio-diesel waste, lowered the glycerol price and started to generate a “glycerol waste problem”. In this respect it is of interest to know that for every tonne (=metric ton) of bio-diesel manufactured via the transesterification process, about 100 kg of glycerol waste is generated. Even though glycerol has traditionally been used in pharmaceuticals, cosmetics, toothpaste, paints and other commercial products, the rapidly developing bio-diesel industry with its large glycerol waste streams created a challenge to find profitably novel uses for this waste. Therefore, metabolically versatile microorganisms capable of generating hydrogen gas from glycerol and glycerol waste streams, such as bio-diesel wastes, could make significant contributions to generate clean bio-hydrogen energy.

And despite the fact that fermentative hydrogen production from corn, other renewable sources of glucose or readily fermentable carbohydrates shows comparatively higher H₂ production rates, it is currently assumed that yields of 8-12 moles H₂ per mole glucose are required to see this approach to become a commercially viable way of bio-hydrogen production. Hydrogen production with the help of bacterial fermentation of glucose is currently limited to a maximum of 4 moles of hydrogen per mole of glucose. Glucose fermentation in most facultative and obligate anaerobic bacteria results in the formation of fermentation end products, such as organic acids (acetate, lactate, butyrate, succinate), ethanol and 2,3-butanediol, most of which drastically lower the pH and are toxic for the bacteria and which bacteria are unable to further convert into hydrogen.

Despite the fact that relatively high hydrogen production rates have been reported for a series of fermentative microorganisms (see the Table A below), including Enterobacter aerogenes E82005 (Tanisho S., et al., Int. J. Hydrogen Energy 12(9): 623-627 (1987)), Enterobacter cloacae IIT-BT08 (Kumar N. & Das D., Process Biochemistry 35: 589-593 (2000)), Klebsiella oxytoca (Minnan L., et al., Res. Microbiol. 156(1):76-81 (2005); Beneman, J. Nature Biotechnol. 14:1101ff (1996)), Thermotoga sp. (Van Niel, E. W. J., et al., Int. J. Hydrogen Energy 27:1391-1398 (2002); Van Ooteghem S. A., et al., Appl Biochem Biotechnol. 98-100:177-89 (2002)), and Clostridium beijerinckii (Taguchi, F. et al., U.S. Pat. No. 5,350,692 (Sep. 27, 1994)), under experimental lab conditions and with purified glucose as the feedstock, there is currently no clear contender for a robust, versatile and industrially capable microorganism, which metabolic activities allow the production of more than 4 moles of hydrogen per mole of glucose. For example, for the high hydrogen producing Enterobacter cloacae species IIT-BT08, Kumar et al. reported a molar conversion efficiency of 2.2 moles of hydrogen per mole of glucose and of 3 moles of H₂ per mole of sucrose. For Enterobacter aerogenes E82005, a fermentative bacterium with the highest known hydrogen evolution rate, Tanisho et al. reported the generation of only 1 mole of hydrogen per mole of glucose with glucose as feedstock.

TABLE A
Comparative H₂ yield and production rates of different microorganisms

| | | | Max. H₂ | |
|---|---|---|---|---|
| | | Max. H₂ | Production | |
| | Max. H₂ Yield | production | Activity | |
| | (mol H₂/mol | Rate | (mmol H₂/g | Author |
| Microorganism | glucose) | (ml H₂/l × h) | dry cell × h) | (Year) |
| Photosynthetic | | | | |
| Chlamydomonas | n/a | 1.6^{#} | | Melis A., et al. |
| reinhardtii | | | | (2001) |
| Oscillatoria sp. Miami | n/a | 8.96^{#} | 0.4^{#} | Phillips E. J. |
| BG7 | | | | (1983) |
| Rhodopseudomonas | n/a | 118.7^{#} | 5.3^{#} | Hillmer P. et al. |
| capsulate | | | | (1977) |
| Rhodospirillum rubrum | n/a | 67.2^{#} | 2.5^{#} | Ormerod J. G. et |
| | | | | al. (1961) |
| Fermentative^{&} | | | | |
| Clostridium butyricum | n/a | n/a | 7.0^{#} | Miyake J. et al. |
| | | | | (1984) |
| Clostridium butyricum | n/a | n/a | 7.3^{#} | Karube I. et al. |
| | | | | (1976) |
| Clostridia sp. | 1.8^{#} | 231^{#} | n/a | Iyer P. et al. |
| | | | | (2004) |
| Citrobacter intermedius | n/a | 246.4^{#} | 9.5^{#} | Brosseau J. D. et |
| | | | | al. (1982) |
| Citrobacter freundii | 1.286 | 90 | n/a | Kumar G. R. et |
| | | | | al. (1989) |
| Klebsiella oxytoca | 1.0 | 87.5 | 9.6 | Minnan L. et al. |
| | n/a^{#} | 350^{#} | 15.2^{#} | (2005) |
| Enterobacter aerogenes | 1.0 | 270 | 11 | Tanisho S. et al. |
| E82005 | 1.1^{#} | 388^{#} | 11^{#} | (1987) |
| | | 470^{#}_{(Sucrose)} | 21^{#}_{(Sucrose)} | |
| Enterobacter cloacae | 2.2 | 447 | 29.5_{(Sucrose)} | Kumar N. et al. |
| IIT-BT08 | 6.0 | 660_{(Sucrose)} | | (2000) |
| Caldicellulosiruptor | 5.9^{#} | 202^{#}_{(Sucrose)} | 11.7^{#}_{(Sucrose)} | Van Niel E. W. J. |
| saccharolyticus | 3.3-3.6^{#} | 448^{#} | 30^{#} | et al. (2002) |
| | | | | deVrije T. et al. |
| | | | | (2007) |
| Thermotoga elfii | 3.3^{#} | 100.8^{#} | 4.5^{#} | Van Niel E. W. J. |
| | | | | et al. (2002) |
| Thermotoga neapolitana | n/a | 460^{#} | n/a | Van Ooteghem S. A. |
| | | | | et al. (2002) |
| Thermoanaerobacterium | 2.2 | 42 | n/a | Shin H. S. et al. |
| thermosaccharolyticum | | | | (2005) |

^{&}= if not indicated otherwise, rate and yield number for glucose as carbon feedstock
^{#}= Numbers reported for pH controlled incubations

Continuous high hydrogen production by known strictly anaerobic hydrogen producing bacteria, such as Clostridia sp. and Thermotoga sp., is hampered by the introduction of oxygen gas, a growth toxin to these microorganisms, usually carried in with the continuously supplied feedstock. Another major obstacle which prevents the successful industrial scale use of hydrogen producing fermentative microorganisms for cost-effective generation of hydrogen gas is the high risk of contamination of the reaction vessel with produced fermentation end products or from introduced toxins and/or growth adverse compounds via the continuously supplied feedstock material, e.g. soil or sludge. For example, the facultative anaerobic bacterium Klebsiella oxytoca shows drastically reduced hydrogen production after prolonged fermentation due to the accumulation of organic acids. Under batch fermentation conditions, this microorganism has been reported to show a maximum hydrogen production rate and yield of 87.5 ml/1 h and 1.0 mol H₂ per 1.0 mol glucose (Minnan L., et al., Res. Microbiol. 156(1):76-81 (2005)). Of all the factors that prevented the successful introduction of bio hydrogen fermentation platforms into the competitive, fossil fuel-dominated energy market, the relatively high costs of the feedstock together with the low hydrogen production rates of the microbes in use were the most significant.

To date only the two strictly anaerobic and thermophilic bacteria Caldicellulosiruptor saccharolyticus and Thermotoga elfii (see Table A) have been reported as having hydrogen production yields of more than 3 moles H₂ per mole of glucose/hexose under optimized continuous flow fermentation conditions. For the hydrogen producing bacterium Thermoanaerobacterium thermosaccharolyticum and most enterobacteria carbohydrate decomposition yields of around 2 mol H₂ per mol of hexose were reported (Shin H. S. & Youn J. H., Biodegradation 16(1): 33-44 (2005) & Table A).

In the past, several strategies have been pursued to significantly maximize the hydrogen production yield and rate of fermentative microorganisms and to push the hydrogen production yield of these contenders close or past the 4 mole H₂ per 1 mole glucose barrier. Efforts to increase H₂ yields of strictly anaerobic and facultative anaerobic bacteria included heat treatment of the sludge or soil inoculum (in case of strict anaerobes), dissolved gas removal, gas sparging and testing of various organic loading rates (OLR) and hydraulic retention times (HRT) of the reaction vessels (Kraemer J. T. & Bagley D. M., Biotechnol. Lett. 29(5): 685-695 (2007)). Gas sparging of the fermentation vessel was shown to increase the H₂ yields compared to un-sparged fermentation vessels by some currently unknown mechanism. Others used genetic and metabolic engineering approaches to improve microbial hydrogen gas production, e.g. by introducing the genes of hydrogen gas evolving hydrogenase enzymes into E. coli and other transformation suitable microorganisms. Morimoto K., et al. (FEMS Microbiology Letters 246: 229-234 (2005)) reported the successful cloning and overexpression of the [Fe]-hydrogenase gene (HydA) from Clostridium paraputrificum into E. coli and achieved a 1.7-fold increased hydrogen production compared with a wild-type strain. Genetic transformation of the cyanobacterium Synechococcus PCC7942 with a hydrogenase gene from Clostridium pasteurianum was reported to lead to an increased hydrogen production (Asada Y., et al.; J. Biosci. Bioeng. 88(1): 1-6 (1999)). Liu et al. used an integrational ‘knock out mutagenesis’ approach to create metabolically engineered mutants of Clostridium tyrobutyricum with an inactivated acetate kinase gene (ack) to improve butyric acid and hydrogen production in this bacterium. The resulting mutant was reported to produce 23.5% more butyrate (0.42 vs 0.34 μg glucose) and 50% more H₂ (0.024 μg) from glucose than the non-mutated version (Liu X. et al., Biotechnol. Prog. 22(5): 1265-1275 (2006)).

In 2006, Penfold D. W. et al. (FEMS Microbiol. Lett. 262(2): 135-137 (2006)) reported increased hydrogen production of the Escherichia coli strain MC4100 and its formate hydrogenlyase upregulated mutant (DeltahycA) after deleting the genes encoding the twin-arginine translocation (Tat) system. The hydrogen production rate of two developed Tat defective mutant strains doubled from 0.88 mL H₂ mg dry weight-1 L culture-1 in the parental strain to around 1.70 mL H₂ mg dry weight-1 L culture. Recently, it was reported that cloning the hoxEFUYH-encoded bidirectional hydrogenase from the cyanobacterium Synechocystis sp. PCC 6803 into E. coli, enhanced the hydrogen yields up to 41-fold and the transformed hoxEFUYH expressing E. coli cells produced twice as much hydrogen as Enterobacter aerogenes HU-101 a known high hydrogen generating bacterium (Maeda T. et al.; BMC Biotechnol. 23(7): 25ff (2007)).

Other studies showed increased hydrogen production yield in an anaerobic microbial fuel (MFC) cell system after augmenting the electrochemical potential of the anaerobes with the help of an externally applied electrode (Liu H., et al.; Environ. Sci. Technol. 39(11): 4317-4320 (2005)). This study showed that additional hydrogen was generated at the cathode from the fermentation waste product acetate after augmenting the electrochemical potential of the anaerobes by applying an electrode-supplied voltage of 250 mV. This MFC system which validity for other bacteria, i.e. facultative anaerobes, and long-term use has not been proven yet was claimed to have the potential to generate between 8-9 mol H₂ per mol glucose. Other approaches showed that removal or lowering of the CO₂ gas in the head space of the fermentation platform leads to increased biological hydrogen gas production (Park W. et al., Environ. Sci. Technol. 39(12): 4416-4420 (2005)). In this study, reduction of the head space CO₂ level in the anaerobic fermentation vessel from 24.5% to a level of 5.2% during the highest gas production phase, increased the hydrogen production yield by 43% from 1.4 to 2.0 mol of H₂ per mol of glucose.

Summarized, a low cost method that significantly increases the hydrogen production yield of hydrogen generating microorganism in the presence of diverse feedstock would make an important contribution to help to overcome technological hurdles that prevented bio hydrogen systems from cost effectively entering the competitive energy market.

Enterobacteria and other fermentative microbes are known to be metabolically very versatile and to generate a huge variety of fermentative end products, such as acetate, 2,3 butanediol, succinate and lactate, from different feedstock. Many of these fermentative end products have high commercial value and only need a low cost process to effectively extract them from fermentation reactions which could make a major contribution for the “green chemistry” industry. For example, the enterobacter end product 2,3-butanediol is an important industrial chemical and is of commercial interest because of its application as a solvent and liquid fuel additive. Butanediol also finds application as a plasticizer, as a humectant and as an important precursor molecule for tetrahydrofuran synthesis, an inert solvent used for numerous polymer and organometallic compound synthesis. The “green chemistry” market is expected to be a huge growth market. According to one study, the US solvents market is expected to grow to $3.4 billion in 2007, with the green solvents sector growing at about six percent annually to make up nearly 25 percent of the overall market.

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

### BRIEF SUMMARY OF THE DISCLOSURE

### I. Method for Microbial Hydrogen Gas Production

The competitive entry of bio-hydrogen generating systems operating with hydrogen gas (H₂) producing microorganisms requires significant improvement of the intrinsically low hydrogen production yields of the biological components. The instant disclosure includes a process or method which helps push the hydrogen production yield of microorganisms in use close to, or past the 4 mole H₂ per mole of glucose barrier. Several strategies and methods have been pursued in the past, from genetic engineering of existing hydrogen generating microbes to optimization of the fermentation platforms in use, but none of these elaborate, biosafety concerning or cost intensive approaches lead to increases in hydrogen gas production rates to much more than 50 percent.

This disclosure describes a simple, safe and low cost process which increases the gas production rate of a recently isolated and characterized bacterium, called Enterobacter sp. SGT06-1™ (patent pending) and of other hydrogen generating microorganisms by more than 250 percent using highly absorptive and low cost materials, most prominently, but not exclusively, diatomaceous earth (SiO₂), metal oxides (TiO₂, SnO₂, CeO₂, Ti/Fe/O, SnO₂:F, Al₂O₃, FeO₃), silicates, zeolites, activated carbon (charcoal), fibrous and microcrystalline cellulose. Many of the materials examined as part of this discovery have a long history of use as filtration materials, e.g. diatomaceous earth, zeolites, activated charcoal, cellulose, and metal oxides have been reported to increase the adhesion of a series of bacteria primarily due to the materials positive net charge as well as pronounced hydrophobicity. The presence of defined amounts of these absorptive materials during the fermentation process using the disclosed bacteria not only triggers increased hydrogen gas production, but also allows the easy and low cost post-fermentative extraction of marketable fermentation products, most prominently but not exclusively lactate, 2,3 butanediol, and succinate, with the help of the disclosed absorptive materials and method.

This disclosure further provides a process which allows low cost collection and extraction of fermentation end- or by-products of Enterobacter sp. SGT06-1™ and/or other microorganisms with the help of the disclosed absorptive materials, most prominently diatomaceous earth. This process is based on the high and reversible absorption of lipophilic and charged molecules to the disclosed absorptive material during the fermentation process using the disclosed microorganisms.

Enterobacter sp. SGT06-1™ has been previously described (U.S. patent application Ser. No. 11/829,599; filed Jul. 27, 2007) to be metabolically very versatile and to generate hydrogen gas from diverse feedstock, including glucose, sucrose, arabinose, cellobiose, maltose, D-xylose, L-rhamnose and D-mannitol. Aspects of the disclosure include the increase in hydrogen production rate of Enterobacter sp. SGT06-1™ from diverse carbohydrates, including glucose, cellobiose, sucrose, maltose and mannitol, in the presence of the disclosed absorptive materials. In other embodiments, a combination of two or more of the disclosed absorptive materials is used to increase the hydrogen production yield and rate of the disclosed microorganisms. A further aspect of the disclosure is a method of culturing a microorganism in the presence of the absorptive materials as described herein under defined cultivation conditions. In other embodiments, a disclosed microorganism is cultured under conditions that allow high production rates of hydrogen gas, such as by use of the carbohydrate(s).

Cultivation conditions used in the disclosed method includes the use of an aqueous based culture medium, or aqueous environment to which the disclosed absorptive material is added in its solid form to form a suspension. In some embodiments, a cultivation condition includes vessel components, such as an impeller, onto which surfaces the absorptive material has been immobilized, e.g. by sputtering or coating, to form thin films. In most cases, the salts of the aqueous culture medium are in milligram or microgram amounts, and the absorptive material is in gram amounts, such as by addition of exogenous salts and absorptive material to a culture medium. Non-limiting examples of the salts include those containing iron, selenium, molybdenum, nickel, magnesium, zinc, copper, borate and/or cobalt. Non-limiting examples of the absorptive materials include crude or purified diatomaceous earth, silica (SiO₂), crystalline or amorphous silicates, e.g. ZrSiO₄, zeolites, activated carbon (charcoal), metal oxides, e.g. titanium dioxide (TiO₂), tin dioxide (SnO₂), cerium dioxide (CeO₂), aluminum oxide (Al₂O₃), zinc oxide, microcrystalline and fibrous cellulose. The absorptive materials may be supplied to the fermentation platform in milli-, micro- or nanogranular, fibrous, or milli-, micro- or nanocrystalline form. In other embodiments, the absorptive materials may be implemented into the fermentation vessels in form of thin films prepared by depositing the absorptive material on suitable inorganic surfaces, e.g. uncoated glass surfaces, by chemical vapor deposition or other methods known in the arts.

A cultivation condition of the disclosure may also include the presence of a gaseous phase above the culture medium. The gas phase may be optionally continuously flushed, or replenished, with a desired gas. The desired gas preferably does not contain oxygen. In some embodiments, the desired gas is a noble gas, such as argon as a non-limiting example.

A cultivation condition of the disclosure also includes a temperature, and a pH level, suitable to achieve optimum effect of the disclosed absorptive materials on increased hydrogen production yield and rate of the microorganism in use. In some embodiments, the temperature is maintained at or below about 45° C. In other embodiments, the pH is maintained at a level from about 4.5 to about 7.5, such as at about 5.0, about 5.5, about 6.0, about 6.5, or about 7.0 or in a range between any of these values.

A cultivation condition of the disclosure may also include the continuous supplying of the absorptive material and of a liquid feedstock, or medium, to the microorganism. In some embodiments, the feedstock contains at least one component selected from monosaccharides, disaccharides, polysaccharides, alcohols, alcoholic sugars, amino acids, fatty acids, and combinations thereof. Non-limiting examples of monosaccharides and disaccharides include glucose, sucrose, maltose, cellobiose, other saccharides containing glucose units, or any combination of the foregoing. A non-limiting example of a suitable alcohol of this invention is glycerol. In some embodiments, a feedstock contains arabinose, xylose, galactose, rhamnose, mannitol or any combination of the foregoing.

So an additional aspect of the disclosure is a culture medium or formulation for use in the method as described herein. The medium or formulation may be a complex or enriched, or alternatively defined or synthetic, growth media which supports the effect of the absorptive material on hydrogen gas production by the used microorganism. In some embodiments, the medium or formulation contains tryptone, peptone, yeast extract or combinations thereof. In some embodiments, the medium or formulation allows optimum increase in hydrogen gas production yield and rate under the conditions used. In other embodiments, the medium or formulation is the defined or synthetic which allows for maximum hydrogen gas production.

In a further aspect, the disclosure includes a method of extracting fermentative microbial by-products or end products with the aid of the disclosed absorptive materials. The method may comprise incubating a hydrogen producing microorganism in the presence of one or more of the disclosed absorptive materials for a defined time period, at a defined temperature and at a defined pH to allow optimum binding of the fermentative products to the absorptive material. After passing of the defined time period, preferentially but not exclusively one to 24 hours, the absorptive material is separated from the growth media and the contained microorganism by spontaneous or enforced gravitational sedimentation of the absorptive material. Then the fermentative products are separated from the absorptive material using suitable extraction media, solvents, ultrasonic waves or other conditions known in the arts. Non-limiting examples suitable for extraction of the products from the absorptive material include the use of diethylether, ethylacetate, dichloromethane, hexane as extraction solvents alone or other methods known in the arts. In some embodiments, the method comprises concentration and identifying the fermentation products associated with and extracted from the absorptive material using chromatographic methods and equipment known in the arts of molecular detection and analysis. Non-limiting examples of fermentation products extractable by the disclosed method include hydrogen gas (H₂), acetate, lactate, succinate, formate, butanediol, and butanol.

### II. Hydrogen Gas Producing Microorganisms

The disclosure is also based in part on the isolation and characterization of a microorganism, referred to as Enterobacter sp. SGT-T4™ herein. The microorganism produces high amounts of hydrogen gas (or molecular hydrogen, H₂) from diverse carbon-made (or carbonaceous) feedstock and belongs to the bacterial family of enterobacteriaceae, a very ubiquitous and versatile group of gram-negative, facultative anaerobic bacteria. Enterobacteria are known to be metabolically versatile and are able to gain cell energy via respiratory (aerobic) or fermentative (anaerobic) degradation of a wide variety of different carbon containing molecules as starting materials. Enterobacteria which commonly occur in soil, water, sewage, food and are also found as normal intestinal inhabitants of humans and animals, are well studied and known to catabolize D-glucose and other carbohydrates, including L-arabinose, cellobiose, maltose, D-xylose, L-rhamnose, D-mannitol, D-sorbitol and trehalose. They are also known to produce organic acids and gas. Some enterobacterial species are known to generate hydrogen gas from other carbon-made molecules, such as pyruvate and glycerol. Glucose can be derived from many sources, but it is very abundant in green plants and in other renewable biomass-derived materials where it usually appears in the form of the disaccharide sucrose and of the polysaccharides starch and cellulose. Other monosugars, most prominently arabinose, xylose, galactose and rhamnose are common components of the hemicellulose and pectin fraction of renewable biomass, e.g., green plants and other phototrophic organisms. Another important renewable biomass-derived component is the 3-carbon molecule glycerol which is an integral compound of plant- or animal-derived oils, lipids and fats.

One aspect of the disclosure includes a hydrogen producing microorganism as described herein. Non-limiting examples of microorganisms of the disclosure includes a microorganism comprising a 16S rDNA sequence fragment represented by SEQ ID No: 1 (Table 5). The disclosure thus includes a microorganism of the enterobacteriaceae family which generates high amounts of hydrogen gas from carbohydrates derived from a diverse range of starch, cellulose, and hemicellulose containing materials, or a combination of two or more of such materials. In some embodiments, a disclosed microorganism of the enterobacteriaceae family utilizes one or more of the carbon containing compounds listed above. In some cases, the microorganism generates large amounts of hydrogen gas and at a high rate from glycerol and glycerol-containing feedstock, for example bio-diesel waste.

In another aspect, the disclosure includes a method of culturing a microorganism as described herein. In some embodiments, the microorganism is cultured with one, two or more carbon containing compound, including one or more carbohydrates as a non-limiting example, under defined cultivation conditions. In other embodiments, the disclosed microorganism is cultured under conditions that allow high production rates of hydrogen gas, such as by use of the carbohydrate(s). The disclosure thus includes a method of producing hydrogen gas by cultivating a disclosed microorganism. In further embodiments, hydrogen gas production is based upon growth of a disclosed microorganism on the glycerol content of waste streams derived from bio-diesel production. In some cases, the glycerol is produced by transesterification or other methods known in the field of producing the bio fuel.

The disclosure includes additional embodiments of a method of culturing a microorganism. In some embodiments, a disclosed microorganism is cultured with alcoholic sugars as feedstock, e.g. mannitol, under defined cultivation conditions. In other embodiments, a disclosed microorganism is cultured under conditions that allow high production rates of hydrogen gas, such as by use of the alcoholic sugars. In further embodiments, hydrogen gas production is based upon extracted alcoholic sugar products of brown algae (kelp) extracts.

In other embodiments, the disclosure includes a method of culturing a microorganism as described herein. In some embodiments, the method comprises use of the tertiary alcohol glycerol as feedstock under defined cultivation conditions. In some embodiments, a disclosed microorganism is cultured under conditions that allow high production rates of hydrogen gas, such as by use of glycerol. In further embodiments, hydrogen gas production is based upon cultivation of the microorganism in the presence of crude, extracted bio-diesel production wastes containing glycerol.

In most embodiments, a cultivation condition used in a disclosed method includes the use of an aqueous based culture medium, or aqueous environment. In some embodiments, a cultivation condition includes the presence of inorganic salts. In some cases, the salts are in milligram or microgram amounts, such as by addition of exogenous salts to a culture medium. Non-limiting examples of the salts include those containing iron, selenium, molybdenum, nickel, magnesium, zinc, manganese, copper, borate and/or cobalt. In other embodiments, a cultivation condition includes the presence of known co-substrates or prosthetic groups of crucial metabolic enzymes and other bio-catalysts. Non-limiting examples include nicotinic acid, nicotine amide, riboflavin, biotin, and/or thiamin, which may be exogenously added to a culture medium for use in a disclosed method. In yet other embodiments, a cultivation condition includes the presence of sulfur-containing compounds. Non-limiting examples include ammonium sulfate, cysteine, methionine, glutathione, N-acetyl cysteine and/or dithiothreitol, which may be exogenously added to a culture medium for use in a disclosed method.

In further embodiments, a cultivation condition includes redox-active compounds and/or compounds with antioxidant chemical characteristics. In some cases, the amount of such a compound is defined in the culture medium. Non-limiting examples of such a compound include ascorbic acid, tocopherols, cysteine, N-acetyl cysteine and/or glutathione.

In yet additional embodiments, a cultivation condition includes the presence of highly absorptive materials, crystals, minerals and/or mineral-like compounds. In some cases, the material is a metallosilicate, such as an aluminosilicate, and the amount of such a material or mineral is optionally defined in the culture medium. In other cases, such a material or mineral is added to the culture medium in granular, microgranular and/or nanogranular form. Non-limiting examples of a highly absorptive material or mineral include cellulose fibers, diatomaceous earth, Celite®, natural zeolite (clinoptilolite), a synthetic zeolite, silicon dioxide, titanium dioxide, zirconium dioxide, and/or cerium dioxide. Of course the disclosed microorganism may also be cultured in a method summarized in section I above.

A cultivation condition of the disclosure may also include the presence of a gaseous phase above the culture medium. The gas phase may be optionally continuously flushed, or replenished, with a desired gas. In some embodiments, the desired gas does not contain oxygen. In other embodiments, the desired gas is a noble gas, such as argon as a non-limiting example. In alternative embodiments, the gas is flushed in a discontinuous manner, such as at defined times, during the culturing of the microorganism with the desired gas. In further embodiments, the desired gas is bubbled through the aqueous environment, or culture medium. The bubbling may be continuous or discontinuous, such as at defined time points during the culturing of the microorganism.

In some embodiments, the introduction of gas may be used to remove carbon dioxide generated by the cultivation conditions. Alternatively, carbon dioxide may be chemically bound to an absorbent present under the cultivation conditions. In some cases, the absorbent is an alkali metal liquid matrix. Non-limiting examples include sodium hydroxide (NaOH), and/or a solid matrix, such as soda lime.

A cultivation condition of the disclosure also includes a temperature, salinity and a pH level (each of which is optionally defined), suitable for the growth and/or propagation of the microorganism as well as hydrogen gas production. In some embodiments, the temperature is maintained at or below about 45° C. In other embodiments, the salinity of the medium is maintained at a concentration of less than 6%. In other embodiments, the pH is maintained at a level as described herein.

In a further aspect, the disclosure includes a method of producing energy. The method may comprise producing hydrogen gas with a disclosed microorganism and supplying the hydrogen gas to a hydrogen gas energy converting device. Non-limiting examples include a fuel cell, gas turbine, internal combustion engine or other suitable hydrogen energy conversion device. The converting device may convert the hydrogen gas to either kinetic energy or potential energy. Kinetic energy is based on motion including that of waves, electrons, atoms, molecules, substances, and objects. Non-limiting examples of kinetic energy include electrical energy, radiant energy, thermal energy, motion energy, and sound. Potential energy is stored energy and the energy of position. Non-limiting examples of potential energy include chemical energy, stored mechanical energy, nuclear energy, and gravitational energy.

In a yet further aspect, the disclosure includes a method of identifying, or detecting a disclosed microorganism. In some embodiments, the method comprises identifying or detecting a microorganism as comprising a 16S rDNA sequence containing a sequence with more than 87% homology to SEQ ID No:1 (Table 5). Non-limiting examples include identifying or detecting a microorganism as comprising a 16S rDNA containing SEQ ID No: 1.

In other embodiments, the method comprises identifying or detecting a microorganism as containing a sequence which is amplified by a pair of primers comprising sequences represented by SEQ ID No: 2 and SEQ ID No: 3 (Table 4). The method may comprise use of the two sequences as the primers in a polymerase chain reaction (PCR) with DNA from a candidate microorganism followed by comparison of the amplified sequence with that amplified from SGT-T4™. Non-limiting examples include comparison of the length or base composition of the amplified nucleic acid, or of the sequence of amplified nucleic acid. Optionally, the method may further comprise assaying the candidate microorganism for hydrogen gas production.

The method of identifying or detecting may be of a candidate microorganism isolated from a naturally occurring source or as it is found in nature. Alternatively, the method may be performed with a candidate microorganism derived from a microorganism disclosed herein. In some embodiments, such a derivative, or mutant, microorganism may be one which occurs with passage of a disclosed microorganism in culture. Alternatively, a derivative microorganism may be the result of intentional mutagenesis of a disclosed microorganism.

In other embodiments, the disclosure includes a method of mutagenizing, or creating, derivative microorganisms from a disclosed microorganism. The method may comprise taking a disclosed microorganism and contacting it with a mutagen. Non-limiting examples of mutagens include mutagenic agents, such as chemical compounds, and radiation. The method may further comprise screening the treated microorganism(s) for an rDNA sequence as described herein and/or production of hydrogen gas. In some embodiments, the screening may comprise detection of increased hydrogen gas production. Non-limiting examples of increased production include an increased rate of production over a given period of time and/or increased total gas production over a given period.

Another aspect of the disclosure includes nucleic acid molecules for use in the methods as described herein. In some embodiments the molecules are isolated from the cellular or genomic DNA environment in which they are normally found. A non-limiting molecule is represented by SEQ ID No: 1 (Table 5). In other embodiments, the molecule may be a vector or plasmid, such as one comprising the molecules represented by SEQ ID No: 1. Other molecules of the disclosure are represented by SEQ ID Nos: 2 and 3 (Table 4).

### III. Production of Biomass

This disclosure is additionally based in part on the discovery that the green microalgae Chlorella protothecoides can be grown to very high cell densities under low cost conditions in a medium supplemented with processed bacterial fermentation waste streams as heterotrophic feedstock. High microalgal biomass can be achieved with the disclosed method under moderate and even dim light conditions, while at the same time producing large quantities of intracellular microalgal oils. This discovery makes a major contribution to pave the way for low cost, sustainable, environmentally friendly and socially more responsible production of bio-diesel fuel to meet the increasing transportation energy needs of an increasing world population. The disclosed invention can be used to produce bio-diesel fuel and other value products from the extracted bio-oils of the green microalgae Chlorella under industrial scale conditions using suitable bio-reactors.

In one example, the microalgae Chlorella protothecoides shows high biomass production and high-yield oil production in aerated or non-aerated batch cultures from processed bacterial fermentation waste as heterotrophic feedstock. In the presence of 0.5 liters of processed bacterial fermentation waste in the growth medium a maximum dry biomass of 5 g of microalgae per liter culture was achieved after 5 days of incubation under batch conditions and an microalgal oil yield of about 0.35 g oil per gram algal dry weight was achieved.

In some embodiments of the disclosure, Chlorella protothecoides responds with high biomass production and produces high amounts of microalgal oils in a medium supplemented with processed bacterial waste streams collected after fermentation of glycerol-containing bio-diesel waste stream utilizing the bacterium Enterobacter sp. SGT-T4 or other disclosed hydrogen producing microorganism. In other embodiments of the disclosure, Chlorella protothecoides shows high biomass production and produces high amounts of microalgal oils in a medium supplemented with processed bacterial waste streams collected after fermentation of carbohydrate-containing materials as feedstock, for example brewery waste, utilizing the bacterium Enterobacter sp. SGT06-1 or other disclosed hydrogen producing microorganism. In further embodiments, a combination of two or more bacterial fermentation waste streams are used as feedstock for Chlorella protothecoides biomass and microalgal oil production.

In another aspect, the disclosure includes a method of culturing the green microalage Chlorella protothecoides in a growth medium supplemented with defined amounts of processed bacterial fermentation waste under batch, chemostat or continuous flow conditions. In some embodiments, the Chlorella protothecoides is cultured with one, or combinations of two or more different bacterial fermentation waste streams under defined cultivation conditions. In other embodiments, Chlorella protothecoides is cultured under conditions that allow high production rates of biomass and microalgal oils at the same time such as by use of processed bacterial fermentation waste streams. The disclosure thus includes a method of producing microalgal biomass and oils by cultivating Chlorella protothecoides. In further embodiments, microalgal biomass and oils production is based upon the presence of defined amounts of ethanol, and/or organic acids in the bacterial waste streams derived from fermentation of bio-diesel refinery wastes for bio-hydrogen production. In further embodiments, microalgal biomass and oils production is based upon the presence of defined amounts of 2,3-butanediol, acetoin, ethanol, and/or organic acids in the bacterial waste streams derived from fermentation of cellulosics- and/or hemicellulosics wastes for bio-hydrogen production. In yet a further embodiment, microalgal biomass and oils production is based upon the presence of defined amounts of urea in the bacterial waste streams derived from fermentation of biomass-derived wastes in the presence of human or animal urine as nitrogen source.

In yet another aspect, the disclosure includes a process to prepare a bacterial fermentation waste stream suitable to serve as low cost feedstock for culturing Chlorella protothecoides for high microalgal biomass and oil production. This process includes collecting a liquid medium at defined time points, preferentially but not exclusively after 24 hours, after fermentation of bio-diesel waste utilizing the bacterium Enterobacter sp. SGT-T4 (or other disclosed hydrogen producing microorganism) using methods as described herein and separating the bacterium from the waste medium using separation methods known in the arts of bacterial separation. In alternative embodiments, fermentation is of carbohydrates, preferentially but not exclusively glucose, cellobiose, maltose, sucrose, xylose, utilizing the bacterium Enterobacter sp. SGT06-1 (or other disclosed hydrogen producing microorganism). In further embodiments, fermentation is of a carbohydrates-containing waste stream, such as brewery waste, utilizing the bacterium Enterobacter sp. SGT-T4 (or other disclosed hydrogen producing microorganism). Additional embodiments include fermentation of a carbohydrates-containing waste stream, such as brewery waste or processed office paper waste, utilizing the bacterium Enterobacter sp. SGT06-1 (or other disclosed hydrogen producing microorganism). Preferred separation methods include, but are not limited to, application of artificial gravitational force, e.g. centrifugation, or filtration.

In yet another aspect, the disclosure includes a process to prepare a bacterial fermentation waste stream suitable to serve as low cost feedstock for culturing Chlorella prothecoides for high microalgal biomass and oil production. This process includes preparing a fermentation waste medium by growing a bacterium, preferentially but not exclusively Enterobacter sp. SGT06-1 or Enterobacter sp. SGT-T4, in a liquid medium in the presence of a suitable feedstock and in the presence of defined amounts of a silicaceous material, preferentially but not exclusively of natural or synthetic zeolites as described herein.

Other embodiments of this disclosure include culturing Chlorella protothecoides as follows. In most embodiments, a cultivation condition used in a disclosed method includes the use of an aqueous based culture medium, or aqueous environment. In some embodiments, a cultivation condition includes the presence of inorganic salts. In some cases, the salts are in gram amounts, such as by addition of exogenous salts to a culture medium. Non-limiting examples of the salts include those containing sodium, potassium, phosphate, and/or sulphate. In some cases, the salts are in milli- or microgram amounts, such as by addition of exogenous salts to a culture medium. Non-limiting examples of these salts include those containing magnesium, iron, molybdenum, zinc, manganese, copper, borate and/or cobalt. In other embodiments, a cultivation condition includes the presence of known co-substrates or prosthetic groups of crucial metabolic enzymes and other bio-catalysts. Non-limiting examples include nicotinic acid, nicotine amide, pantothenic acid, riboflavin, biotin, and/or thiamin, which may be exogenously added to a culture medium for use in a disclosed method. In yet other embodiments, a cultivation condition includes the presence of nitrogen-containing compounds or solutions. Non-limiting examples include ammonium sulfate, urea, nitrate, glycine, glutamate, cysteine, animal or human urine, which may be exogenously added to a culture medium for use in a disclosed method.

Additional embodiments of this disclosure include culturing Chlorella protothecoides as follows. In these embodiments, a cultivation condition used in a disclosed method includes the use of an aqueous based culture medium, or aqueous environment, as described above which is supplemented in a defined ratio with processed bacterial fermentation waste. In some embodiments, a cultivation condition includes a preferred culture medium-bacterial fermentation waste ratio of 50:50 (v/v). In yet other embodiments, cultivation conditions include culture medium-bacterial fermentation waste ratios of 15:85 (v/v), 25:75 (v/v), 35:65 (v/v), 45:55 (v/v), 55:45 (v/v), 65:35 (v/v), and 75:25 (v/v).

In another embodiment of this disclosure, the processed bacterial fermentation waste stream is continuously supplied to the microalgal culture medium at defined time intervals to assure continuous and maximum microalgal biomass and oil production.

A cultivation condition of the disclosure may also include the continuous presence of a gas or gas mixture in the culture medium. The gas may be optionally or continuously bubbled into the culture medium, or alternatively flushed, or replenished, into the gas phase above the culture medium. In some embodiments, the desired gas is carbon dioxide as a non-limiting example. In yet another embodiment, the gas is derived from a commercial gas reservoir or supplied from a gas-emitting process, such as a fermentation vessel or bio-reactor. In alternative embodiments, the gas is flushed into the gas phase a discontinuous manner, such as at defined times, during the culturing of the microalgae with the desired gas. In further embodiments, the desired gas is bubbled through the aqueous environment, or culture medium in a continuous or discontinuous manner, such as at defined time points during the culturing of the microalgae.

A cultivation condition of the disclosure also includes a defined temperature, salinity and a pH level, suitable for the optimum growth and oil production of the microalgae from the supplied bacterial fermentation wastes as heterotrophic feedstock. In other embodiments, the temperature of the culture medium containing the microalgae is maintained at preferentially but not exclusively 25° C. In other embodiments, the pH is maintained at a level from about 4.5 to about 7.5, such as at about 5.0, about 5.5, about 6.0, about 6.5, or about 7.0 or in a range between any of these values.

Another embodiment of this disclosure includes the harvesting of the microalgae from the culture medium at defined time points, preferentially but not exclusively at 4 days, 5 days or 6 days, for microalgal oil extraction.

And a further embodiment of this disclosure is a method to effectively disrupt the microalgal cell integrity for quantitative microalgal oil extraction. For this, microalgae are harvested after a certain time period, preferentially but not exclusively 5-6 days, of cultivation in a suitable cultivation medium in the presence of processed bacterial fermentation waste and concentrated by methods known in the arts, for example centrifugation of filtration. The concentrated microalgae are dried, reconstituted in small volumes of distilled water and subsequently milled under low temperature conditions, e.g. in the presence of defined amounts dry ice, and in the presence of defined amounts of silicaceous materials, preferentially but not exclusively diatomaceous earth or Celite (silicon dioxide). The microalgal oils in the thus prepared cell lysates are extracted by methods known in the arts of lipid/oil extraction, for example with the help of chloroform, n-hexane, and/or mixture thereof.

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1a shows the comparative time-dependent total gas production of the bacterium Enterobacter sp. SGT-T4™ in comparison to the known gas producing enterobacteria Enterobacter sp. SGT06-1™ and Enterobacter aerogenes ATCC 13048. In this study, the bacteria under investigation were incubated at 37° C. in test tubes containing inverted Durham tubes filled with complex growth medium (10 ml) containing peptone and 2.5% glucose as feedstock. Gas production of the bacteria was measured and plotted as mm trapped gas in the inverted Durham tubes over time.

FIG. 1b shows the comparative time-dependent total gas production of the bacterium Enterobacter sp. SGT-T4™ in different growth media in the presence or absence of natural zeolite. In this study, SGT-T4™ bacteria were incubated at 37° C. in test tubes containing inverted Durham tubes filled with 10 ml of either peptone-glucose (PG) medium (=Medium 1) or with 10 ml tryptone-yeast-glucose (TYG) medium (=Medium 2). Gas production of the bacteria over time was measured and plotted as mm trapped gas in the inverted Durham tubes. The concentration of glucose and zeolite (Zeo) in the media was 2.5% in this study.

FIG. 2a shows the time-dependent hydrogen gas generation of the bacterium Enterobacter sp. SGT-T4™ in TYG medium in the presence or absence of natural zeolite. In this study, SGT-T4™ bacteria were incubated at 37° C. under microaerobic conditions in rubber-stoppered flasks filled with 50 ml of tryptone-yeast-glucose (TYG) medium. Gas production of SGT-T4™ over time was measured with the help of a liquid-gas exchange method consisting of an upside-down graduated cylinder filled with a 15% NaOH solution that trapped the carbon dioxide fraction of the evolved gas. In this study the concentration of glucose and zeolite (Zeo) in the media was 2% and 2.5%, respectively.

FIG. 2b shows the time-dependent hydrogen gas production rate of the bacterium Enterobacter sp. SGT-T4™ calculated in ml H₂ evolved per hour per liter growth medium. SGT-T4™ was incubated under the same conditions as described in FIG. 2a. in TYG medium in the presence or absence of natural zeolite.

FIG. 3a shows the time-dependent total gas production of Enterobacter sp. SGT-T4™ in the presence of the monosaccharides glucose, xylose, arabinose, galactose or of the disaccharides maltose and sucrose as feedstock. In this study, SGT-T4™ was incubated at 37° C. in test tubes containing inverted Durham tubes filled with peptone growth medium (10 ml) with 2.5% of the carbohydrates as feedstock. Gas production was measured and plotted as mm trapped gas in the inverted Durham tubes over time.

FIG. 3b shows the time-dependent total gas production of Enterobacter sp. SGT-T4™ in the presence of the alcoholic sugars mannitol or sorbitol or of the polyhydroxyalcohol glycerol as feedstock. In this study, SGT-T4™ was incubated at 37° C. in test tubes containing inverted Durham tubes filled with peptone growth medium (10 ml) with 2.5% of the feedstock. Gas production was measured and plotted as mm trapped gas in the inverted Durham tubes over time.

FIG. 4 shows the time-dependent total gas production of Enterobacter sp. SGT-T4™ with industrial glycerol or crude bio-diesel waste (BDW) as feedstock in the presence or absence of zeolite (Zeo) in the growth medium. In this study, SGT-T4™ was incubated at 37° C. in tryptone-yeast (TY) medium (10 ml) in the presence of either 300 mM glycerol or 0.8 ml of crude bio-diesel waste (BDW) as carbon feedstock. Gas production was measured in the presence or absence of 2.5% of zeolite material in the growth medium and plotted as mm trapped gas in the inverted Durham tubes over time. The known high gas production of Enterobacter sp. SGT-T4™ with glucose as feedstock is shown for comparison.

FIG. 5a shows the time-dependent hydrogen gas (H₂) production of Enterobacter sp. SGT-T4™ with 300 mM industrial glycerol or 3.8 ml of crude bio-diesel waste (BDW) as carbon feedstock in the presence or absence of 2.5% zeolite (Zeo) in the growth medium. In this study, SGT-T4™ bacteria were incubated at 37° C. under microaerobic conditions in rubber-stoppered flasks filled with 50 ml of tryptone-yeast (TY) medium. Gas production of SGT-T4™ over time was measured with the help of a liquid-gas exchange method consisting of an upside-down graduated cylinder filled with a 15% NaOH solution that trapped the carbon dioxide fraction of the evolved gas. The time-dependent H₂ production of Enterobacter sp. SGT-T4™ with 2.5% glucose as feedstock is shown for comparison.

FIG. 5b shows the time-dependent hydrogen production rates (in ml H₂ per hour per liter) of Enterobacter sp. SGT-T4™ with 300 mM industrial glycerol or with 3.8 ml of crude bio-diesel waste (BDW) as carbon feedstock in the presence or absence of 2.5% zeolite (Zeo) in the growth medium. The SGT-T4™ bacteria were incubated at 37° C. under the same conditions as described in more detail in FIG. 5a. The hydrogen production rate of glucose (2.5%) as feedstock in the absence of zeolite is shown for comparison.

FIG. 6a shows the increased gas production of Enterobacter sp. SGT-T4™ with pre-processed bio-diesel waste solution (BDWS) in comparison with crude bio-diesel waste (BDW) as feedstock in the presence or absence of zeolite (Zeo) in the growth medium. In the case of BDWS, SGT-T4™ was incubated at 37° C. in 5 ml 2× tryptone-yeast (TY) medium to which 5 ml of pre-processed bio-diesel waste solution (BDWS) as carbon feedstock was added. Pre-processed bio-diesel waste solution (BDWS) was prepared by dissolving 40 ml of pre-cleared bio-diesel waste (BDW) in 460 ml deionized water followed by pH-neutralization with 6N HCl. In the case of BDW as feedstock, 0.8 ml of crude bio-diesel waste (BDW) was directly added to 9.2 ml tryptone-yeast (TY) medium and the time-dependent accumulation of gas in the Durham tubes measured. Gas production was measured in the presence or absence of 2.5% of zeolite (Zeo) material in the growth medium and plotted as mm trapped gas in the inverted Durham tubes over time. The known high gas production of Enterobacter sp. SGT-T4™ with glucose as feedstock in the presence of 2.5% zeolite is shown for comparison.

FIG. 6b shows the effect of increasing concentration of zeolite (Zeo) material in the growth medium on the gas production of Enterobacter sp. SGT-T4™ with pre-processed bio-diesel waste solution (BDWS) as feedstock. In this study, SGT-T4™ was incubated at 37° C. in 5 ml 2× tryptone-yeast (TY) medium to which 5 ml of pre-processed bio-diesel waste solution (BDWS) was added as carbon feedstock. Pre-processed bio-diesel waste solution (BDWS) was prepared by dissolving 40 ml of pre-cleared bio-diesel waste (BDW) in 460 ml deionized water followed by pH-neutralization with 6N HCl. The accumulation of gas in the inverted Durham tubes in the absence or in the presence of defined amounts of zeolite (Zeo) material was measured after 7 hours incubation time and plotted as mm gas in the Durham tubes.

FIG. 7a shows the time-dependent hydrogen production rates (in ml H₂ per hour per liter) of Enterobacter sp. SGT-T4™ with pre-processed bio-diesel waste (BDWS) as feedstock in the presence or absence of 2.5% zeolite (Zeo). In this study, SGT-T4™ bacteria were incubated at 37° C. under microaerobic conditions in rubber-stoppered flasks filled with 25 ml of 2×-concentrated tryptone-yeast (TY) medium and 25 ml BDWS solution. Gas production of SGT-T4™ over time was measured with the help of a liquid-gas exchange method consisting of an upside-down graduated cylinder filled with a 15% NaOH solution that trapped the carbon dioxide fraction of the evolved gas. The BDWS solution was prepared by dissolving 40 ml of pre-cleared bio-diesel waste (BDW) in 460 ml of sterile distilled water followed by pH neutralization with HCl.

FIG. 7b shows the maximum hydrogen production rates, volumes and yield of Enterobacter sp. SGT-T4™ (in the presence or absence of 2.5% zeolite) in comparison to the published rates, volumes and yield of Enterobacter aerogenes HU-101 [Ito T., et al., J. Biosci. Bioeng. 100(3): 260-265 (2005)] and Klebsiella pneumonia DSM2026 [Liu F. & Fang B. Biotechnol. J. 2(3): 374-380 (2007)] with bio-diesel waste as feedstock. The numbers for Enterobacter sp. SGT-T4™ and Klebsiella pneumonia were from incubations of the microbes under batch conditions, while the shown numbers for Enterobacter aerogenes HU-101 result from continuous culturing of self-immobilized cells in a 60 ml packed-bed reactor (asterisk). The shown hydrogen gas volumes for the individual bacteria are given for a 50 ml fermentation volume and after 24 hours incubation at 37° C.

FIG. 8 shows the comparative sedimentation behavior of Enterobacter sp. SGT-T4™, Enterobacter sp. SGT06-1™ and Enterobacter aerogenes ATCC13048 (E.a.) in tryptone-yeast-glucose (TYG) medium. 15 hour cultures of the microorganisms were resuspended in the media by gentle shaking of the tubes and then left on the bench for 12 hours without further agitation of the tubes during this time period. The picture was taken after 12 hours.

FIG. 9 shows the concentration-dependent increase in total gas production of the microorganism Enterobacter sp. SGT 06-1™ in the presence of increasing amounts of diatomaceous earth (DE) measured as gas accumulation (in mm) in an inverted Durham tube. For this study, the bacterium was incubated at 37° C. in a Durham test tube under batch conditions in 10 ml of complex growth medium containing 2.5% cellobiose as carbon source. The amount of gas accumulated in the inverted Durham tube was measured after 10 hours incubation time.

FIG. 10a shows the time-dependent total gas production of Enterobacter sp. SGT 06-1™ in the absence (solid lines) or presence (dashed lines) of 1% diatomaceous earth (DE) with glucose (filled squares), xylose (filled diamonds) and mannitol (filled triangles) as feedstock. FIG. 10b shows the time-dependent total gas production of Enterobacter sp. SGT 06-1™ in the absence (solid lines) or presence (dashed lines) of 1% diatomaceous earth (DE) with sucrose (filled circles), maltose (filled squares) and cellobiose (filled diamonds) as feedstock. For both studies, the bacterium was incubated at 37° C. in a Durham test tube under batch conditions in complex growth medium containing 2.5% of the different carbohydrates under investigation.

FIG. 11 shows the time-dependent hydrogen production of Enterobacter sp. SGT 06-1™ in the absence (solid lines) or presence (dashed lines) of 1.5% of the absorptive materials diatomaceous earth (DE) and zeolite (Zeo) monitored as fuel cell voltages in volts (V). For this study, SGT 06-1™ was incubated at 37° C. in growth medium either with 2.5% sucrose (filled squares) or 2.5% maltose (filled diamonds) as carbon source under batch conditions.

FIG. 12 shows the comparative total gas production of the microorganisms Enterobacter sp. SGT 06-1™ (SGT06-1; solid squares), Enterobacter aerogenes ATCC 13048 (E. aerogenes; solid triangles), Enterobacter cloacae ATCC 15361 (E. cloacae; solid diamonds) and Citrobacter freundii ATCC 13316 (C. freundii; solid circles) in the absence (solid lines) or presence (dashed lines) of 1% diatomaceous earth (DE) in the culture medium. The upper panel shows the time-dependent total gas production and the lower panel shows the comparative total gas production of the different bacteria in the absence (black bars) or presence (dotted bars) of 1% diatomaceous earth (DE) after 10 hours incubation time. For this study, the bacteria were incubated at 37° C. in Durham test tubes filled with growth medium and in the presence of 2.5% glucose as carbon source.

FIG. 13a shows the comparative total gas production of Enterobacter sp. SGT 06-1™ in the absence (solid line) or presence (dashed lines) of different absorptive materials. The absorptive materials under investigation were diatomaceous earth (DE) (upper panel; filled squares), Zeolite (Zeo) (upper panel; filled triangles); Celite®545 (CT) (upper panel; filled circles), AquaPerl (AqP) (upper panel; open diamonds), activated charcoal (ACC) (upper panel; crosses), fibrous cellulose (fCel) (lower panel; open circles), microcrystalline cellulose (mCel) (lower panel; open triangles) and polyvinylpolypyrrolidone (PVP) (lower panel; open squares). No gas was produced in the absence of Enterobacter sp. SGT06-1™ with DE in the test tube (upper panel; open squares). Gas production of Enterobacter sp. SGT 06-1™ in the absence of absorptive material in the cultivation tube is shown as control in the upper and lower panels (filled squares with white cross). FIG. 13b shows the comparative total gas production of the Enterobacter sp. SGT06-1™ in the absence (white bar) or presence (grey bars) of the absorptive materials Celite®545 (CT), Zeolite (ZT), diatomaceous earth (DE), AquaPerl (AqP), activated charcoal (ACC), microcrystalline cellulose (mCel), fibrous cellulose (fCel) and polyvinylpolypyrrolidone (PVP) after 8 hours incubation time. For this study, the bacterium was incubated at 37° C. in Durham test tubes filled with complex growth medium containing 2.5% glucose as carbon source and 1% of absorptive material.

FIG. 14 shows the total gas production of the microorganism Enterobacter sp. SGT 06-1™ in the absence (solid lines) or presence (dashed lines) of 1% diatomaceous earth (DE) in the culture medium with brewery waste filtrate (BWF) as carbon feedstock. The gas production with maltose (1.5%) as carbon source is shown for comparison. For this study, the bacteria were incubated at 37° C. in Durham test tubes filled with 5 ml of twice concentrated complex growth medium and 5 ml of BWF (solid circles) or 5 ml of a 3% maltose solution (solid diamonds). Sterile brewery waste filtrate was received after incubation of freshly collected brewery malt waste (30%) in potassium phosphate buffer in the presence of calcium chloride and amylase enzyme. After incubation for 8 hours at 30° C. under constant stirring, the brewery waste was processed using a series of filtration steps and sterile filtered.

FIG. 15 shows the effect of increasing volumes of processed bacterial fermentation waste (BFW) on cell growth and biomass production of the green microalga Chlorella protothecoides. A 5-6 day culture of C. protothecoides (0.5 ml) was inoculated in 4× concentrated heterotrophic (HT) growth medium (1.25 ml) in the presence of 0.5 ml (10%), 1.25 ml (25%), 2.5 ml (50%) and 3.75 ml (75%; v/v) of processed bacterial fermentation waste (BFW) or with 1% (w/v) of glucose as heterotrophic control feedstock. The final volume in the test tubes was adjusted to 5 ml with sterile distilled water. For the phototrophic control culture, a 5-6 day C. protothecoides culture (0.5 ml) was inoculated in phototrophic Proteose growth (PT) medium (4.5 ml). If not indicated differently in the Figure, all cultures were grown in natural light conditions, at a temperature of 26° C., under occasional aeration. Samples (2 ml) were taken from the cultures at day 6, diluted with 3 ml of distilled water and the absorbance was measured with a spectrophotometer at a wavelength of 550 nm. Processed bacterial fermentation waste (BWF) used in this experiment was prepared as following. Bacterial fermentation waste was collected from a 24 hour culture of the bacterium Enterobacter sp. SGT-T4 after fermentation of glycerol-containing bio-diesel refinery waste in the presence of zeolite (2.5%) in the fermentation vessel. The collected fermentation waste was centrifuged for 30 minutes at 3,350 rpm to remove the bacteria and the resulting supernatant was sterile filtered using a 0.2 μm pore size filtration device (115 ml filter unit, SFCA, Nalgene).

FIG. 16 shows light microscopic images depicting the increased cell size and number of Chlorella protothecoides grown in the presence of bacterial fermentation waste (BFW) as heterotrophic feedstock. A 5-6 day culture of C. protothecoides (0.5 ml) was inoculated in 4× concentrated heterotrophic (HT) growth medium (1.25 ml) in the presence of either 40% (v/v) bacterial fermentation waste (2.5 ml) (right panel) or 1% (w/v) glucose as heterotrophic control feedstock (middle panel). The final volume in these test tubes was adjusted to 5 ml with sterile distilled water. For the phototrophic control culture (left panel), a 5-6 day C. protothecoides culture (0.5 ml) was inoculated in heterotrophic (HT) growth medium without feedstock. In this study, all cultures were grown in natural dim light conditions, at a temperature of 26° C., under occasional aeration. Samples (10-15 μl) were taken from the cultures at day 6 and observed through a light microscope (Olympus CX41, Japan) at 1000-times magnification power using immersion oil. Pictures were taken with the help of a microscope-mounted Microfire™ digital camera (Optronics, U.S.A.). The pictures were captured and analyzed using Pictureframe™ software. Processed bacterial fermentation waste (BWF) used in this experiment was prepared as following. Bacterial fermentation waste was derived from a 24 hour culture of the bacterium Enterobacter sp. SGT-T4 after fermentation of glycerol-containing bio-diesel refinery waste in the presence of zeolite (2.5%) in the fermentation vessel. The collected fermentation waste was centrifuged to remove the bacteria and the resulting supernatant was sterile filtered using a 0.2 μm pore size filtration device.

FIG. 17 shows the comparative growth of Chlorella protothecoides under phototrophic or heterotrophic conditions in the presence of glucose or bacterial fermentation waste as feedstock. A 5-6 day C. protothecoides culture (10 ml) was inoculated in heterotrophic growth medium in the presence of either 50% (v/v) processed bacterial fermentation waste (T4FW) or 1% glucose as heterotrophic feedstock. A third flask was inoculated with a 5-6 day C. protothecoides culture (10 ml) and the microalga was grown under phototrophic conditions (−) in Proteose growth medium (90 ml). The algae cultures (100 ml total volume) in all three flasks were grown under continuously aerated conditions and under natural dim light conditions and at a temperature of 26° C. The flasks were continuously aerated under sterile conditions with the help of an air pump. Samples (1 ml) were taken from all three cultures every 24 hours until day 8, diluted in 4 ml of distilled water and the absorbance was measured with a spectrophotometer at a wavelength of 550 nm. Bacterial fermentation waste (T4FW) was collected from a 24 hour culture of the bacterium Enterobacter sp. SGT-T4 after fermentation of glycerol-containing bio-diesel refinery waste. The collected fermentation waste was centrifuged to remove the bacteria and the resulting supernatant was filtered through a 0.2 μm sterile filtration device.

FIG. 18 shows the effect of processed bacterial fermentation waste obtained after fermentation of glucose or bio-diesel refinery waste by different enterobacteria on cell growth of the green microalga Chlorella protothecoides. A 5-6 day culture of C. protothecoides (0.5 ml) was inoculated in 4× concentrated heterotrophic (HT) growth medium (1.25 ml) in the presence of 2 ml (40% v/v) of processed bacterial fermentation waste derived from either Enterobacter sp. SGT06-1 (06Glc-FW), Enterobacter sp. SGT-T4 (T4Glc-FW, T4BDW-FW) or Enterobacter aerogenes ATCC13048 (EaBDW-FW) cultures cultivated in the presence of either glucose (Glc) or biodiesel waste (BDW) as feedstock. The final volume in the test tubes was adjusted to 5 ml with sterile distilled water. For the phototrophic control culture, a 5-6 day C. protothecoides culture (0.5 ml) was inoculated in heterotrophic growth medium (5 ml) without feedstock. If not indicated differently in the Figure, all cultures were grown in natural dim light conditions, at a temperature of 26° C., under occasional aeration. Samples (2 ml) were taken from the cultures after day 6 of cultivation, diluted in 3 ml of distilled water and the absorbance was measured with a spectrophotometer at a wavelength of 550 nm. Processed bacterial fermentation waste used in this experiment was prepared as following. Bacterial fermentation waste was derived from 24 hour cultures of the bacteria Enterobacter sp. SGT06-1 (06), Enterobacter sp. SGT-T4 (T4) and Enterobacter aeogenes ATCC 13048 (Ea) after fermentation of glucose (06Glc, T4Glc) or glycerol-containing bio-diesel refinery waste (T4BDW, EaBDW) in the presence of zeolite (2.5%) in the fermentation vessel. The collected fermentation waste was centrifuged to remove the bacteria and the resulting supernatant was sterile filtered using a 0.2 μm pore size filtration device.

FIG. 19 shows the comparative biomass production of Chlorella protothecoides under phototrophic or heterotrophic cultivation conditions in the presence of glucose or bacterial fermentation waste as feedstock. A 5-6 day C. protothecoides culture (10 ml) was inoculated in heterotrophic growth medium in the presence of either 50% (v/v) processed bacterial fermentation waste (HT+T4FW) or 1% glucose (HT+Glucose) as heterotrophic feedstock. A third flask was inoculated with a 5-6 day C. protothecoides culture (10 ml) and the microalga was grown under phototrophic conditions (PT−) in Proteose growth medium (90 ml). The algae cultures (100 ml total volume) in all three flasks were grown under continuously aerated conditions, under natural dim light conditions and at a temperature of 26° C. The flasks were continuously aerated under sterile conditions with the help of an air pump. After 5 days cultivation in the corresponding culture media, a 50 ml sample was taken from each flask. The samples were centrifuged for 15 minutes at 3,300 rpm to sediment the algal cells. After decanting of the supernatants, the algal pellets were dried for 2 days in a dry oven at a temperature of 42° C. to determine the dry biomass of the samples. Bacterial fermentation waste (T4FW) was collected from a 24 hour culture of the bacterium Enterobacter sp. SGT-T4 after fermentation of glycerol-containing bio-diesel refinery waste. The collected fermentation waste was centrifuged to remove the bacteria and the resulting supernatant was filtered through a 0.2 μm sterile filtration device.

FIG. 20 shows the comparative microalgal oil production of C. protothecoides after cultivation in processed bacterial fermentation waste. A 5-6 day culture of C. protothecoides (10 ml) was inoculated in 4× concentrated heterotrophic (HT) growth medium (25 ml) in the presence of 50 nm of processed bacterial fermentation waste. The final volume in the cultivation flask was adjusted to 100 ml with sterile distilled water. For the phototrophic control culture, a 5-6 day C. protothecoides culture (10 ml) was inoculated in phototrophic Proteose (PT) growth medium (90 ml). Both cultures were grown in natural dim light conditions, at a temperature of 26° C., under continuous aeration. A 50 ml sample was taken from each flask after 6 days cultivation and centrifuged to collect the algal cells. The cell pellet was dried, reconstituted in 1 ml of distilled water and the microalgal oils were extracted from the cells using conventional porcelain mortar and pistil method in the presence of dry ice and defined amounts of Celite or diatomaceous earth. The oils were extracted from the cell lysate by conventional n-hexane extraction known in the arts. The collected n-hexane fractions were slowly evaporated in the presence of the lipid/oil indicator dye Sudan IV. Pictures of the test tubes showing the carmine red oil droplets received after Sudan IV reaction were taken after complete evaporation of the hexane fraction.

### DETAILED DESCRIPTION OF THE DISCLOSURE

### IV. Method for Microbial Hydrogen Gas Production

General

A significant obstacle that has hindered the successful and competitive use of microorganisms for industrial scale bio-hydrogen generation was the low hydrogen production rates and yields of the currently favored bacteria. To the extent known, and prior to the instant disclosure, no studies exist that show the rate- and yield-increasing effect of silicates, most prominently zeolites, on bacterial hydrogen production, even though many different strategies have been tried in the past to significantly increase the low hydrogen production rates and yield of bacteria. These include process optimization and genetic engineering. Zeolites are quite common crystalline aluminosilicate minerals with more than forty natural zeolites known today. Clinoptilolite, a naturally occurring zeolite and the most researched of all natural zeolites, has a cage-like structure consisting of SiO₄ and AlO₄ tetrahedra which are joined by shared oxygen atoms. Since the negative charges of the AlO₄ units of zeolites are balanced by the presence of exchangeable cations, usually sodium, potassium, calcium, magnesium, and iron, which can be easily replaced by other ions, zeolites possess high cation exchange and ion absorptive capacity. Despite their diverse known roles as filter material, absorbants and chemical catalysts, this disclosure shows a novel function of zeolites as cheap, abundant and very effective bacterial hydrogen production rate and yield increasing material.

The instant disclosure includes a simple and cheap method based on the use of common absorbent materials such as diatomaceous earth, zeolites and cellulose that increase the hydrogen production rate of hydrogen gas evolving bacteria. The disclosure is expected to make a significant contribution to the demanded switch from a fossil fuel economy to a hydrogen gas-based economy. Cost effective bio hydrogen production from renewable and/or waste feedstock with the help of high hydrogen evolving microorganisms using the disclosed process could make significant contributions to conserve existing landfill space, create a new energy infrastructure, provide health, environmental, climate and economic benefits and reduce reliance on finite fossil fuel supplies.

Summarized, a simple, low cost method and process that dramatically improves the currently applied extraction methods, most importantly of the high energy input requiring distillation process, could make a significant contribution to help overcome the current technological hurdles that prevented bio hydrogen systems from cost effectively entering the energy market. The disclosed method, which is based on the known high absorptive behavior of diatomaceous earth, silicates, metal oxides, microcellulosics and other absorptive materials is expected to allow future low cost industrial scale extraction of commercially interesting fermentative end products. Even though many applications of silicious adsorbents have been described in the past, including the clarification and filtration of industrial liquids, treatment of hydrocarbons, such as petroleum or vegetable oil, for the purpose of absorptive purification, filtration and decolorizing of gases, no process has been described to use these absorptive materials for the purpose of enhancement of hydrogen production yield of hydrogenic microorganisms. Exemplary documents relating to the use of silicious materials as adsorbents, including some relating to diatomaceous earth adsorbents, include the following U.S. Pat. Nos. 1,555,639, 1,598,255, 1,634,514, 1,992,547, 2,044,341, 2,701,240 and 5,603,836.

The disclosure includes a low cost method that dramatically increases the hydrogen production yield of hydrogen generating microorganisms from diverse feedstock in the presence of different absorptive materials is described. The method is expected to make a significant contribution to help overcome technological hurdles that to date prevented bio-hydrogen energy systems from cost effectively entering the competitive energy market. The disclosure may help to begin the demanded switch from a fossil fuel economy to a clean hydrogen gas-based green economy. Cost effective bio hydrogen production from renewable and/or waste feedstock with the help of high hydrogen evolving microorganisms using the disclosed method could make significant contributions to conserve existing landfill space, create a new energy infrastructure, improve economic, public health, environmental and climate benefits and ultimately reduce global reliance on finite fossil fuel supplies.

Enterobacteria and other fermentative microbes are known to be metabolically very versatile and to generate a huge variety of fermentative end products, such as acetate, 2,3 butanediol, succinate and lactate, from different feedstock. Many of these fermentative end products have high commercial value and only need a low cost process to effectively extract them from fermentation reactions which could make a major contribution for the “green chemistry” industry. For example, the enterobacter end product 2,3-butanediol is an important industrial chemical and is of commercial interest because of its application as a solvent and liquid fuel additive. Butanediol also finds application as a plasticizer, as a humectant and as an important precursor molecule for tetrahydrofuran synthesis, an inert solvent used for numerous polymer and organometallic compound synthesis. The “green chemistry” market is expected to be a huge growth market. According to one study, the US solvents market is expected to grow to $3.4 billion in 2007, with the green solvents sector growing at about six percent annually to make up nearly 25 percent of the overall market.

This disclosure includes a simple, low cost method and process that improves currently applied extraction methods, such as the high energy input requiring distillation process. The simple and cheap extraction method which is based on the known high absorptive behavior of diatomaceous earth, zeolites, silicates, activated carbon (charcoal), metal oxides, microcellulosics and other absorptive materials is expected to allow future low cost industrial scale extraction of commercially interesting fermentative end products.

The disclosure is the result of extensive screening efforts to find suitable materials and conditions to further increase the already high hydrogen gas production rate of a recently isolated and characterized microorganism, i.e. Enterobacter sp. SGT06-1™ (patent pending; U.S. patent application Ser. No. 11/829,599). The disclosure includes the discovery and description of diatomaceous earth (DE) and other highly absorptive materials as abundant and low cost materials which dramatically increase the total gas and hydrogen gas production rates not only of Enterobacter sp. SGT06-1™, but also of a series of other known hydrogen generating microorganism, including Enterobacter aerogenes, Enterobacter cloacae and Citrobacter freundii.

As disclosed herein, hydrogen producing bacteria, including Enterobacter sp. SGT 06-1™, respond with a more than two times higher gas production rate in the presence of diatomaceous earth and other absorptive materials not only with glucose as fermentative carbon source but also with the carbohydrates xylose, cellobiose, maltose, sucrose, and mannitol (see Table B). Stated differently, the disclosed absorptive materials, most prominently DE, are capable of significantly increasing the hydrogen gas production rates of different hydrogen producing microorganisms and in the presence of different fermentative feedstock.

TABLE B
Comparative hydrogen production rates of Enterobacter sp.
SGT06-1 ™ in the presence or absence of diatomaceous earth
(DE) with different carbohydrates as feedstock for fermentation

| | Gas production rate | Gas production rate | |
|---|---|---|---|
| | without DE | with DE (1%) | % Rate |
| Carbohydrate | [ml gas/h × l] | [ml gas/h × l] | increase |
| Glucose | 301 | 696 | 2.31 |
| Sucrose | 286 | 811 | 2.83 |
| Cellobiose | 261 | 731 | 2.8 |
| Maltose | 119 | 365 | 3.06 |
| Xylose | 91.3 | 420 | 4.6 |
| Mannitol | 221 | 563 | 2.54 |

Without being bound by theory, and offered to improve the understanding of the instant disclosure, it is believed that the disclosed hydrogen gas production rate-enhancing effect of the highly absorptive materials, most prominently diatomaceous earth, on a series of microorganisms is due to absorptive removal of otherwise toxic fermentation end products by the disclosed materials. The disclosed method and bacteria together with the herein described increased fuel cell voltages in the presence of the absorptive materials under fermentation conditions may be used for long term and industrial scale generation of hydrogen gas in combination with known or future energy conversion technologies, i.e. fuel cells and/or gas turbines.

The method and microorganisms described herein contribute to the technical field of bio-energy generation from diverse renewable biomass-derived degradation products, most prominently glucose, xylose, sucrose, maltose, cellobiose and mannitol. The disclosed method allows the generation of hydrogen gas at high rates in the presence of not only the monosaccharide glucose, but also from other important biomass-derived mono- or disaccharides such as xylose, sucrose, maltose and cellobiose. It further allows high rate microbial hydrogen gas generation from mannitol, an alcoholic sugar which is an abundant and important storage molecule found in marine brown algae. The gas production rate-enhancing effect in the presence of absorptive materials, most prominently with diatomaceous earth, is also observed with processed brewery waste as a complex feedstock. The gas production rate-enhancing effect of the disclosed absorptive materials is not only restricted to the patent pending microorganism SGT06-1™, but is also observable with other fermentative microorganisms, i.e. Enterbacter aerogenes, Enterobacter cloacae and Citrobacter freundii. The disclosed methods are extendable and applicable with other known hydrogen producing microorganisms, e.g. Clostridia sp., Thermotogae sp., and the like.

The microbial gas production rate-enhancing method of this disclosure can be effectively implemented into existing or future bio-hydrogen energy systems to cost effectively generate electricity or heat with the help of system-coupled hydrogen fuel cell technology of hydrogen turbines. The disclosure is expected to make significant contributions to decentralized energy generation, energy cost savings, air quality improvement, natural resource conservation, land use protection and pollution prevention.

Absorptive Materials

As described herein, the disclosure includes a method that increases the total gas and hydrogen production rate of microorganism, including members of the enterobacteriaceae family, in the presence of defined amounts of absorptive materials during the fermentation process. Non-exclusive examples of absorptive materials usable for the method are Celite®545, diatomaceous earth (DE), silica (SiO₂), silicates (SiO₄), metal oxides, cellulose, activated carbon (charcoal), carbon nanotubes and other absorptive carbonaceous materials. Non-exclusive examples of silicates are mineral zeolites, i.e. aluminosilicates, olivine, feldspars, nepheline, vermiculite and epidote. The silicates may be a ring silicate, a chain silicate, a sheet silicate or a framework silicate. In some embodiments, however, a silicate of the disclosure is not glass or glass beads. In other embodiments, an absorptive material of the disclosure is not activated carbon (or activated charcoal), such as granules or particles (optionally those with a size of 0.6 to 1.1 mm in diameter) or cylinders (optionally with a diameter of 3 to 4 mm and a height of 10 mm). Non-exclusive examples of metal oxides are titanium oxide (TiO₂), ilmenite (FeTiO₃), titanite (CaTiSiO₅), tin oxide (SnO₂), cerium oxide (CeO₂), SnO₂:Sb, and aluminum oxide (Al₂O₃).

The dimension of the absorptive materials should be small enough, ideally in the micro- or nanometer scale, to create a sufficiently large surface-to-volume ratio for maximum absorption of charged and/or lipophilic molecules. Ideally, but not exclusively, the absorptive materials will be added to the microbial fermentation system in fibrous, amorphous, crystalline, microcrystalline or nanocrystalline form, or combinations thereof. Ideally, the absorptive material will be in purified solid form but may also be added to the microorganism in liquid or gel-sol form to achieve the disclosed gas production rate enhancing effect. Alternatively, the absorptive material is exposed to the fermentation vessel in form of thin films, e.g. metal oxide thin films, coated to suitable surfaces, e.g. glass or ceramic surfaces, by methods known in the arts, such as sputtering or chemical vapor deposition.

Increased Hydrogen Gas Production and Use

The disclosed method includes culturing a microorganism with a suitable medium in the presence of the absorptive materials under conditions to increase the microbial gas production rate. The disclosure also includes a method of culturing a microorganism as described herein to produce hydrogen gas with the help of an in line coupled fuel cell system.

The disclosure thus includes a cell culture comprising a microorganism of the disclosure and a culture medium or formulation as described herein which includes defined amounts of an absorptive material, preferentially but not exclusively diatomaceous earth (DE). In some embodiments, the medium or formulation includes the combination of a source of an absorptive material, of a carbohydrate(s), of a nitrogen source and one or more inorganic salts. In further embodiments, the cell culture may be exposed to an absorbent for carbon dioxide as described herein. The microorganism(s) in a culture of the disclosure may be immobilized or attached, such as on an absorptive material as described herein (as a non-limiting example), or be free (or in suspension) in a liquid culture. In some cases, the free or suspended cells may be in the form of a clump or aggregate, such as a floc. In other embodiments, the culture may be part of a packed-bed reactor, without or without a support material. Non-limiting examples of a support material include lignocellulosic materials, agar, agarose, gels and other suitable materials known to the skilled artisan (see for example Chang et al. Int. J. Hydrogen Energy, 27:1167-1174, 2002; Zhu et al., Int. J. Hydrogen Energy 24:305-310, 1999; Kumar et al. Enz. Microb. Technol., 29:280-287, 2002; and Yokoi et al., J. Ferment. Bioeng., 83:481-484, 1997).

The cell culture may be maintained or propagated under conditions that include a combination of a gaseous phase above the medium or formulation, a suitable temperature, suitable agitation of the medium or formulation and an acceptable pH, each as described herein. In some cases, the gaseous phase comprises an inert or noble gas, which is optionally bubbled through a liquid medium or formulation. Non-limiting examples of a suitable temperature include at or below about 45° C. or about 40° C., about 37° C., about 35° C., about 30° C., or about 25° C.

The disclosure further includes a method of producing energy that comprises releasing energy from hydrogen gas produced by a disclosed method. In some embodiments, the method may comprise delivery of hydrogen gas produced by a disclosed microorganism and supplying the hydrogen gas to a hydrogen gas energy converting device. In some cases, the hydrogen gas releases energy during combustion in the presence of oxygen to form water. In other cases, the energy release occurs via electrochemical conversion, such as in a fuel cell with hydrogen gas as a fuel and oxygen as the oxidant.

Extraction Methods

The disclosure further includes a method of using the disclosed absorptive material, preferentially but not exclusively diatomaceous earth (DE), as a means to extract charged and/or lipophilic molecules from a fermentation vessel containing one or more of the disclosed microorganism. The method may comprise taking a disclosed microorganism and incubating it for a defined time period in the presence of the defined amounts of the absorptive material to allow optimum binding of fermentation by- or end products to the material. Non-limiting examples of fermentation by- or end products being extractable by this method are acetate, formate, succinate, lactate, butanol and butanediol.

In one example, the absorptive material including the bound molecules are allowed to spontaneously sediment to the bottom of the fermentation vessel with the help of gravitational force. There the absorptive material including the bound molecules are removed from the fermentation vessel using means and tools known in the arts of biofermentation. In another example the absorptive material including the bound molecules are removed from the fermentation vessel with the help of forced gravitation or filtration. Following removal of the absorptive material from the fermentation vessel, the absorbed molecules are extracted from the absorptive material using solvents and method known in the arts.

In another example, the absorptive material is vortexed in the presence of one or more of an organic solvent, preferentially but not exclusively, dichloromethane or ethylacetate for a defined time period to allow removal of the absorbed molecules from the absorptive material. In yet another example the absorptive material is vortexed in the presence of one or more of an aqueous solution with a defined pH, preferentially but not exclusively, sulfuric acid, carbonic acid, sodium hydroxide, for a defined time period to allow removal of the absorbed molecules from the absorptive material.

In yet another embodiment the various fermentation products are monitored with the help of detection devices known in the arts of molecular analysis. In one example, the extracted fermentation products are monitored using a coupled HPLC/refractory index detector analysis system equipped with a cation-exchange column as immobile phase and diluted sulfuric acid as mobile phase.

Additional Methods for Hydrogen Gas Production and Use

In addition to disclosed methods for culturing a disclosed microorganism with a suitable medium and conditions to propagate it, the disclosure also includes a method of culturing a microorganism as described herein to produce hydrogen gas. In some embodiments, the microorganism is cultured with a source of carbohydrate(s) as described herein. The method may also comprise cultivation conditions that are suitable or advantageous to hydrogen gas production, such as the use of a culture medium and/or conditions as described herein.

The disclosure thus includes a cell culture comprising a microorganism of the disclosure and a culture medium or formulation as described herein. In some embodiments, the medium or formulation includes the combination of a source of carbohydrate(s), one or more inorganic salts, a processed protein extract, yeast extract, a sulfur-containing compound, and a redox-active compound and/or antioxidant compound, each of which is as described herein. In further embodiments, a cell culture may contain defined amounts of one or a combination of absorptive materials, for example cellulose, cellulose-derivatives, natural zeolites (clinoptilolite), synthetic zeolites, diatomaceous earth, or other alumino- or metal silicates, and may be exposed to an absorbent for carbon dioxide as described herein. In further embodiments, a cell culture may contain defined amounts of one or a combination of solid catalytic materials, for example a natural zeolite (clinoptilolite), synthetic zeolites, or other alumino- or metal silicates, and the bio-reactor containing the catalytic materials may be exposed to a form of electromagnetic energy, for example to visible or UV light.

A cell culture may be maintained or propagated under conditions that include a combination of a gaseous phase above the medium or formulation, a suitable temperature, suitable agitation of the medium or formulation, suitable osmolarity, suitable salt concentration and an acceptable pH, each as described herein. In some cases, the gaseous phase comprises an inert or noble gas, which is optionally bubbled through a liquid medium or formulation. Non-limiting examples of a suitable temperature include at or below about 45° C. or about 40° C., about 37° C., about 35° C., about 30° C., or about 25° C.

The disclosure further includes a method of producing energy that comprises releasing energy from hydrogen gas produced by a disclosed method. In some embodiments, the method may comprise delivery of hydrogen gas produced by a disclosed microorganism and supplying the hydrogen gas to a hydrogen gas energy converting device. In some cases, the hydrogen gas releases energy during combustion in the presence of oxygen to form water. In other cases, the energy release occurs via electrochemical conversion, such as in a fuel cell with hydrogen gas as a fuel and oxygen as the oxidant.

The disclosure thus includes a method of producing molecular hydrogen (H₂) by culturing a disclosed microorganism under conditions allowing hydrogen production. In some embodiments, the conditions include an aqueous environment containing gram amounts of added alkali phosphates, yeast extract, malt extract, and/or a protein hydrolysate extract. Non-limiting examples of a protein hydrolysate extract include tryptone and peptone. In other embodiments, the conditions include an aqueous environment containing milli- or microgram amounts of added inorganic salts, such as calcium, magnesium, manganese, iron, selenium, molybdenum, nickel and/or zinc, or any combination thereof. In further embodiments, the conditions include an aqueous environment containing defined amounts of redox-active compounds and/or compounds with either antioxidant or oxidant chemical characteristics, such as ascorbic acid, N-acetyl cysteine, methionine, cysteine, glutathione, and/or hydrogen peroxide.

In additional embodiments, the conditions include a gas phase above an aqueous environment that is continuously flushed with gas, optionally of a defined amount or composition. In some cases, the gas is a noble gas, such as argon as a non-limiting example. Further embodiments include flushing over time, such as at defined time points, with gas (optionally of a defined amount or composition). Non-limiting examples include flushing with a noble gas like argon as a non-limiting example. Alternatively, the conditions may include an aqueous environment that is continuously bubbled with gas of a defined amount or composition, such as with a noble gas like argon as a non-limiting example. As a further alternatively, the conditions may include an aqueous environment that is flushed with gas of a defined amount or composition, such as a noble gas like argon as a non-limiting example.

In further embodiments, the conditions may include maintaining a culture environment at a constant or relatively constant temperature. In some cases, the temperature may be about 45° C. or below. In other cases, the temperature may be about 40° C. or below, about 37° C. or below, about 35° C. or below, about 30° C. or below, about 25° C. or below, or about room temperature or below.

Additional embodiments include conditions with a continuously supplied liquid feedstock. Non-limiting examples include feedstock derived from a monosaccharide, a disaccharide, a polysaccharide, an alcoholic sugar, a polyhydroxyalcohol, an amino acid, a fatty acid, and any combination thereof. In some cases, a mono- or di-saccharide is selected from glucose, sucrose, maltose, cellobiose and/or other saccharides containing a glucose unit or a combination thereof. In other cases, the feedstock contains arabinose, xylose, galactose, rhamnose, sorbitol and/or mannitol or any combinations thereof. In additional cases, the feedstock contains a polyhydroxyalcohol, such as glycerol, monoacylglycerol and/or diacylglycerol, or a combination thereof as non-limiting examples.

Embodiments of the disclosure further include conditions with generation of carbon dioxide in the culture environment. In some cases, the carbon dioxide is chemically bound or sequestered by inclusion of an alkali metal liquid, or solid, matrix in the environment. Non-limiting examples of a matrix include sodium hydroxide (NaOH) in solution and soda lime as a solid matrix.

### V. Hydrogen Gas Producing Microorganisms

General

The disclosure is also based in part on extensive investigations on an ideal source of hydrogen producing microorganisms and systematically screened guts dissected from different termite (white ant) species for the presence of metabolically versatile and high hydrogen producing microorganisms. The disclosure includes the successful isolation and characterization of a suitable candidate bacterium, termed Enterobacter sp. SGT-T4™. The isolated bacterium has favorably fast growth rates and generates very high amounts of hydrogen gas from glucose as carbon feedstock and also from other renewable biomass-derived carbonaceous molecules, such as glycerol, cellobiose, maltose, sucrose, arabinose, xylose, galactose, rhamnose and alcoholic sugars, such as mannitol and sorbitol. Stated differently, the disclosed microorganism is capable of generating hydrogen gas not only from starch and cellulosics-degradation products, such as glucose, maltose and cellobiose, or from hemicellulosics-derived monosugars, such as rhamnose, xylose, galactose and arabinose, but also from glycerol and alcoholic sugars. Without being bound by theory, and offered to improve the understanding of the disclosed embodiments, the microorganism generates hydrogen gas by fermentation of degradation products of cellulosics materials, such as paper and cotton waste streams, from hemicellulosics degradation products, such as green plant biomass, from alcoholic sugars, such as mannitol, the predominant storage sugar form in brown algae (phaeophytes) and from glycerol, a key component of biological lipids and fats and a major waste product of bio-diesel processing system. The already high hydrogen production rate of the disclosed bacterium with glucose, glycerol, and other carbonaceous feedstock can be further enhanced in the presence of highly absorptive and catalytically active materials, most prominently zeolite and diatomaceous earth, although another metallosilicate may be used.

One non-limiting example of a metallosilicate is a crystalline aluminosilicate, such as a zeolite. More than forty natural zeolites are known and contemplated for use in the practice of the disclosed methods and compositions. One non-limiting example is clinoptilolite,

Also without being bound by theory, the isolated and characterized hydrogen gas-generating bacterium, termed SGT-T4™, is believed to belong to the genus Enterobacter. The bacterium, and derivatives thereof, as well as the cultivation conditions using absorptive materials as described herein, may be used for long term and large scale generation of hydrogen gas in combination with known or future energy conversion technologies, i.e. fuel cells and/or gas turbines.

The microorganisms and methods described herein contribute to the technical field of bio-energy generation from renewable biomass-derived molecules and components, i.e. sucrose, starch, glycerol, alcoholic sugars, as well as cellulose- and/or hemicellulose-containing materials. Throughout this document, cellulose-containing materials are herewith referred to as cellulosics, e.g. paper waste, card board, cotton-made fabrics. Hemicellulose-containing materials, e.g. food processing wastes, agriculture and forestry plant biomass, will be termed hemicellulosics. The disclosed microorganism generates hydrogen gas in the presence of structurally diverse carbohydrates, including the monosaccharides glucose, mannose, xylose, arabinose, galactose, rhamnose, from the disaccharides cellobiose, maltose and sucrose, from the alcoholic sugar mannitol, from glycerol and also from glycerol-containing bio-diesel production wastes.

The disclosed bacteria and processes are suitable for utilization of sucrose, starch, cellulosics and hemicellulosics-derived carbohydrate feedstock, as well as waste streams rich in alcoholic sugars, e.g., mannitol, or glycerol, e.g. bio-diesel production wastes, for industrial scale bio-hydrogen gas production. Proposed industrial scale biohydrogen energy production systems will utilize the microorganism SGT-T4™, or a derivative thereof, at sites with traditionally large starch, cellulosics, hemicellulosics, and glycerol-containing waste loads, such as food processing industries, breweries, large office buildings, government offices, educational institutions, shopping malls, hospitals, farms, nurseries, and bio-diesel processing plants. The microorganism may also be utilized for industrial bio-hydrogen production from sources rich in alcoholic sugars, such as brown algae, and at sites with high amounts of alcoholic sugar-containing waste streams containing mannitol and/or sorbitol. The microorganism, cultivation methods and processes of the disclosure can be effectively used for on-site, decentralized industrial scale production of bio-energy in the form of electricity and/or heat from renewable materials under ultra-low green house gas-emitting conditions. Therefore, the disclosed invention is expected to make significant contributions to domestic energy security, air quality improvement, natural resource conservation, land use protection and pollution prevention.

Microorganisms

As described herein, the disclosure includes a microorganism belonging to the enterobacteriaceae family. The bacterium generates high amounts of hydrogen gas (H₂) from sucrose, different starch, cellulosics- and hemicellulosics-derived carbohydrates, namely glucose, maltose, cellobiose, xylose, rhamnose, galactose and arabinose, and glycerol in different culture media and under defined cultivation methods. In some embodiments, the hydrogen gas produced by the microorganism SGT-T4™ is directly used in a bio-reactor-coupled fuel cell system for long-term electricity generation under ambient temperatures. Because the microorganism only generates hydrogen and carbon dioxide gas from the supplied carbonaceous feedstock and does not release potentially noxious gases, such as hydrogen sulfide (H₂S) and carbon monoxide (CO). Both of these are known to cause fuel cell membrane poisoning. Because of these properties the microorganism SGT-T4™ is ideal for use in combination with fuel cell energy systems.

One non-limiting example of a disclosed microorganism is a hydrogen gas producing microorganism comprising a 16S rDNA sequence represented by SEQ ID No:1. This microorganism is termed SGT-T4™, and it has been isolated from a naturally occurring source to be free of other microorganisms found with it in nature. The genetic material of the microorganism may be further isolated and sequenced by methods known to the skilled person to identify additional sequences that are unique, or specific, to the microorganism and/or hydrogen gas production. These additional sequences may also be used to identify or characterize additional hydrogen gas producing microorganisms of the disclosure.

Additional microorganisms of the disclosure include derivatives, or mutants, of SGT-T4™, such as those which occur spontaneously with passage or cultivation. In some cases, derivative microorganisms may be considered progeny microorganisms of SGT-T4™. In other cases, the derivative microorganisms are spontaneous mutants containing genetic changes at one or more locations in the genomes of SGT-T4™. Non-limiting examples of genetic changes includes insertion and/or deletion of sequences, and/or substitution of one or more base residues. In many embodiments, the derivative or mutant microorganisms retains the hydrogen gas production phenotype of SGT-T4™ and/or a 16S rDNA sequence as described herein.

Whether a derivative, a mutant, or isolated, a microorganism of the disclosure may be identified as comprising a 16S rDNA sequence containing SEQ ID No:1 (Table 5), or a sequence with more than 87% identity or homology to SEQ. ID No:1. In other embodiments, the microorganism comprises a 16S rDNA sequence containing a sequence with more than 87%, more than 88%, more than 89%, more than 90%, more than 91%, more than 92%, more than 93%, more than 94%, more than 95%, more than 96%, more than 97%, more than 98%, or more than 99% identity to SEQ ID No:1. Of course some microorganisms may comprise SEQ ID No:1. Percent identity or homology between two sequences may be determined by any suitable method as known to the skilled person. In some embodiments, a PSI BLAST search, such as, but not limited to version 2.1.2 (Altschul, S. F., et al., Nucleic Acids Rec. 25:3389-3402, 1997) using default parameters may be used.

Identification of Microorganisms

The disclosure includes a method of identifying, or detecting a disclosed microorganism based on the nucleic acid sequences of the microorganism, optionally in combination with the detection of hydrogen production by the microorganism. Thus in some embodiments, the method comprises identifying or detecting a candidate or test microorganism as comprising 16S rDNA containing a sequence with more than 87% identity or homology to SEQ. ID No:1, which identifies it as a microorganism of the disclosure. Microorganisms with such levels of sequence identity are described herein, and they include a microorganism comprising a 16S rDNA containing SEQ ID No:1.

In other embodiments, the method comprises identifying or detecting a microorganism as containing a 16S rDNA sequence which hybridizes to SEQ ID No:1 under “stringent conditions.” Hybridization refers to the interaction between two single-stranded nucleic acids to form a double-stranded duplex molecule. The region of double-strandedness may be full-length for both single stranded molecules, full-length for one of the two single stranded molecules, or not full-length for either of the single-stranded nucleic acids. “Stringent conditions” refer to hybridization conditions comprising, or equivalent to, 68° C. in a solution consisting of 5×SSPE, 1% SDS, 5×Denhardt's reagent and 100 μg/ml denatured salmon sperm DNA followed by washing in a solution comprising 0.1×SSPE, and 0.1% SDS at 68° C., or the above conditions with 50% formamide at 42° C. Stringent condition washes can include 0.1×SSC to 0.2×SSC, 1% SDS, 65° C., for about 15-20 min. A non-limiting example of stringent wash conditions is 0.2×SSC wash at 65° C. for about 15 minutes (see, Sambrook et al., Molecular Cloning—A Laboratory Manual (2nd ed.) Vol. 1-3, Cold Spring Harbor Laboratory, Cold Spring Harbor Press, NY, 1989, for a description of the SSC buffer). Other exemplary stringent conditions include 7% SDS, 0.25 M sodium phosphate buffer, pH 7.0-7.2, 0.25 M sodium chloride at 65° C. to 68° C. or such conditions with 50% formamide at 42° C.

The test or candidate microorganism may be isolated from a naturally occurring source or as found in nature. Alternatively, the method may be performed with a progeny microorganism derived from SGT-T4™. Alternatively, a derivative microorganism may be the result of intentional mutagenesis of a disclosed microorganism.

Mutagenesis Methods

The disclosure includes a method of mutagenizing, or creating, derivative microorganisms from a disclosed microorganism, such as SGT-T4™. The method may comprise taking one of the disclosed microorganisms and treating it with a mutagen. Non-limiting examples of a mutagen include UV or ionizing irradiation, a deaminating agent (such as nitrous acid), an alkylating agent (such as methyl-N-nitrosoguanidine (MNNG)), sodium azide, an intercalating agent (such as ethidium bromide), or phage, transposon or group II intron-mediated mutagenesis. In some embodiments, the method may further comprise the screening of the treated microorganism with a method described herein for the identification of microorganisms by detection of 16S rDNA sequences and/or recA sequences, optionally in combination with detection of hydrogen gas production.

In further embodiments, the mutagenesis method may be used to generate mutated, or altered, microorganisms for identification of microorganisms with increased production of hydrogen gas, relative to SGT-T4™, as a phenotype. Thus the method may comprise contacting a disclosed microorganism with a mutagen and then screening the treated microorganism for increased hydrogen gas production in comparison to SGT-T4™. Non-limiting examples of increased production include an increased rate of production over a given period of time and/or increased total gas production over a given period.

The disclosure also includes a method of genetically engineering a disclosed microorganism. In some embodiments, the method includes genetically engineering a disclosed microorganism, such as SGT-T4™, by transformation of the microorganism with the use of one or more DNA-, RNA- or PNA-based vehicles, such as a plasmid or a bacteriophage. Additional embodiments further include screening a transformed microorganism for increased hydrogen production rates and/or output.

Additional Microorganisms

Additional microorganisms of the disclosed method include other hydrogen gas generating enterobacteria and derivatives, or mutants, of Enterobacter sp. SGT 06-1™, such as those which occur spontaneously with its passage or cultivation. In some cases, a derivative microorganism may be considered a progeny microorganism of SGT 06-1™. In other cases, the derivative microorganism is a spontaneous or induced mutant containing genetic changes at one or more locations in the genome of SGT 06-1™. Non-limiting examples of induced genetic changes includes treatment with a mutagen, insertion and/or deletion of gene sequences, and/or substitution of one or more base residues of critical genes. In many embodiments, the derivative or mutant microorganism retains the high hydrogen gas production rate phenotype of SGT 06-1™ in the presence of the disclosed absorptive materials.

### VI. Production of Biomass

General

Microalgae, such as the green algae Chlorella protothecoides, are ideal candidate organisms for sustainable oil production because of a series of advantages over currently favored terrestrial crop plants. Like plants, microalgae use sunlight to produce oils but they can produce more biomass per time on less space than crop plants, such as palm oil, canola, soybeans and corn. They have a high photosynthesis efficiency leading to higher biomass production and faster growth compared to agricultural crops which are currently used for oil production. They store much more oil per dry weight than currently used agricultural plants. For example, the green microalgae C. protothecoides, stores more than 50% of its weight as extractable oils, more than double the highest oil content of oil palms. Algae have relatively simple growth requirements and grow in low cost composition media. They only need water, sunlight and carbon dioxide, and require significantly less nitrogen as fertilizer than agricultural plants. They are metabolically very versatile.

For example, the microalgae C. protothecoides is able to grow not only under phototrophic conditions, i.e. in the presence of light, but also under heterotrophic conditions, i.e. in the absence of light but presence of glucose and other carbon molecules. Heterotrophic cultivation of Chlorella protothecoides with glucose as carbon feedstock has been reported to result in the accumulation of a much higher microalgal oil contents (>50%) compared with phototrophic growth conditions. However, currently supplied feedstock for heterotrophic growth and high oil production of Chlorella protothecoides are too costly and compete with human food production, therefore preventing its successful economical use.

In order to commercially exploit these very attractive heterotrophic high oil producing properties of Chlorella and to utilize the extracted microalgal oils for economical industrial bio-diesel production, it is important to reduce the cost and source of the microalgal feedstock. As disclosed herein, processed bacterial fermentation waste streams are used as low cost carbon- and nitrogen-containing feedstock to achieve high microalgal biomass and oil production. The fermentation waste is generated after bacterial conversion of waste streams, such as high glycerol-containing bio-diesel refinery waste, for commercial bio-hydrogen generation. Since in recent years, increasing global bio-diesel production from agricultural crop and vegetable oil became more costly, partially due to rising fertilizer and transportation costs, it makes the herewith introduced microalgal oil production from low cost fermentation wastes a very attractive alternative for future bio-diesel fuel generation. This positive outlook for the concept of using microalgal oils instead of oil derived from crops to make bio-diesel is further strengthened by the recently observed negative societal impact of the vegetable oil-based bio-diesel industry on global human food supply. The recent years have seen a dramatically increasing use of prime quality vegetable oils for bio-diesel production which reduced the availability of these oils for human nutrition and food production triggering social unrest and political instability in many parts of the world.

The instant disclosure shows that the green microalgae Chlorella protothecoides produces high biomass and accumulates about 35% of its biomass as oil within its cells in the presence of low cost microbial fermentation waste streams containing ethanol-, organic acids- and human urine. The disclosed methods, implemented into a suitable bio-reactor environment, allow economical generation of quantitative amounts of microalgal oils usable for several purposes, such as industrial scale bio-diesel production.

The disclosure is based in part on investigations to find economical uses for liquid waste streams generated by the bacteria Enterobacter sp. SGT-T4 and Enterobacter sp. SGT06-1 after fermentation of biomass-derived waste streams for commercial bio-hydrogen production. The disclosure includes the use of processed liquid bacterial fermentation wastes as feedstock for heterotrophic cultivation of microalgae, preferentially but not exclusively, the microalgae Chlorella protothecoides (UTEX strain #25). In the presence of defined volumes of the bacterial fermentation waste feedstock supplemented to the algal culture medium, the microalgae shows very high biomass and microalgal oil production within less than 6 days cultivation time. Heterotrophic microalgal biomass production in the presence of bacterial fermentation waste in the culture medium is 20-times higher than compared with growth of Chlorella in standard culture Proteose Bristol medium under phototrophic conditions and 8-times higher than Chlorella growth in the presence of 1% glucose as heterotrophic carbon feedstock. Without being bound by theory, and offered to improve the understanding of the disclosed embodiments, the microalgae Chlorella protothecoides uses some of the supplied fermentation end products of the bacterium SGT-T4, most prominently ethanol, lactic acid and other organic acids, as suitable carbon source for heterotrophic growth and microalgal oil production.

Enterobacteria, to which the fermentation waste producing bacterium Enterobacter sp. SGT-T4 belongs to, are reported to generate high amounts of ethanol, lactate and other organic end products from glycerol- and glycerol-containing bio-diesel waste streams (Ito T., et al., Hydrogen and ethanol production from glycerol-containing wastes discharged after bio-diesel manufacturing process. J. Bioscience & Bioengineering 100(3): 260-265 (2005)). Ethanol has been reported to be a suitable, growth-stimulating carbon source for the green microalgae Chlorella vulgaris (Street H. E., et al., Ethanol as a carbon source for the growth of Chlorella vulgaris. Nature 182(4646): 1360-1 (1958)). The increased growth rate and biomass production of Chlorella protothecoides in the presence of liquid SGT-T4 fermentation waste as feedstock is further accompanied by significantly larger Chlorella cell sizes and much higher intracellular microalgal oil production. The observed high microalgal biomass- and oil production from bacterial fermentation wastes under the described cultivation conditions is expected to make a significant contribution to the field of bio-fuels production from renewable biomass.

The microalgae and growth process described herein may be used for low cost, high efficiency and more ecological production of the oil feedstock for industrial scale bio-diesel production. In this respect one needs to understand that currently the main oil feedstock for bio-diesel production are oils extracted from agricultural plants, most prominently soybean, palm oil, rapeseed and corn. Oil feedstock production from the above agricultural plants is low efficiency due to the low oil content of the used seeds and fruits (usually around 5-20%), require long growth periods (usually a couple of months if not years) and occupy large agricultural areas, often accompanied with the destruction of natural, species-diverse habitats. In contrast, microalgal oil production, especially under heterotrophic conditions, is high efficiency and can be performed under controlled and automated production conditions in bio-reactor environments.

The disclosed heterotrophic microalgal growth and oil production from bacterial fermentation waste as low cost feedstock also has the advantage of very short cultivation periods, with maximum biomass- and oil production achieved in less than 7 days (not months). Finally, due to the high oil production of the microalgae Chlorella under the disclosed conditions, which is about 7-8 times more than in commonly used oil plants, less land will have to be occupied for microalgal oil production. A recent study suggested that the land required for microalgal oil generation is only 0.5% compared with soybeans to produce equal amounts of oil (Chisti Y. Biodiesel from microalgae. Biotechnol. Adv. dio: 10.1016/j.biotechadv.2007.02.001).

The microalgae and method described herein contribute to the technical field of renewable oil production for cost-effective and sustainable bio-fuels, i.e. bio-diesel, generation. The method described herein is of further importance in helping to find novel ways to use waste streams generated from biofuels production processes. The disclosed microorganism and method allows the production of microalgal oils from low cost fermentation waste streams.

Preparation of Bacterial Fermentation Waste

Processed bacterial fermentation waste suitable as heterotrophic, low cost microalgal feedstock may be prepared as follows. A suitable microorganism, such as one disclosed herein, is grown for a defined time period, preferentially but not exclusively 24 hours or more, in a defined growth medium in the presence of a suitable feedstock, preferentially but exclusively bio-diesel refinery waste or a sugar-containing broth, under conditions as described herein. Preferred microorganisms of this invention suitable for generation of bacterial fermentation waste are members of the enterobacteriaceae family, preferentially but not exclusively Enterobacter sp. SGT-T4, Enterobacter sp. SGT06-1, Enterobacter aerogenes, Enterobacter cloacae, Citrobacter freundii, Klebsiella oxytoca.

The microorganism may be cultivated in defined growth medium in the presence or absence of defined amounts of a silicaceous material, preferentially but not exclusively a natural or synthetic zeolite, activated charcoal, or any other absorptive material. The material may be supplied to the fermentation medium in granular, macro-, milli-, micro- or nano-particular size. The growth medium used for fermentation contains, preferentially but not exclusively, following ingredients in grams, milligrams or micrograms per liter: one, two or more phosphate-containing compounds, preferentially but not exclusively mono- or dibasic potassium phosphate, a yeast-derived extract, a peptide-containing extract, preferentially but not exclusively tryptone or peptone, a nitrogen-containing compound or solution, preferentially but not exclusively ammonium sulfate, urea, or urine, a magnesium-containing compound, a calcium-containing compound, a iron-containing compound, a salt, e.g. sodium chloride, and defined volumes of a solution containing following trace elements: nickel, molybdenium, borate, cobalt, selenium, zinc.

A carbonaceous feedstock is supplied to the growth medium for metabolic conversion by the microorganisms. Suitable feedstock of this invention for bacterial fermentation are preferentially but not exclusively glucose, sucrose, fructose, glycerol, xylose, arabinose, rhamnose, galactose, mannitol, sorbitol, maltose, cellobiose, or solutions containing any of the before mentioned feedstock or combinations thereof. After fermentation of the supplied feedstock, e.g. a bio-diesel refinery waste, by a microorganism, the growth medium is collected at defined time points, preferentially but not exclusively after 24 hours. The microorganism will be separated from the growth medium by separation methods known in the arts, preferentially but not exclusively by centrifugation for example for 30 minutes at 3,300 rpm. The resulting cell-free supernatant is collected and sterilized by method known to the skilled person, preferentially but not exclusively by filtering the supernatant with the help of a filtration device possessing a defined pore size, preferentially but not exclusively a pore size of 0.2 μm (micron).

Microorganisms for Biomass Production

The fermentation waste streams suitable for this disclosure derive from fermentation of biomass-derived materials as feedstock as described in more detail in the previous section utilizing suitable microorganisms. Microorganisms usable to prepare the fermentation waste streams belong preferentially, but not exclusively, to the enterobacteriaceae family. Within the family of enterobacteriaceae especially the genera Enterobacter, Citrobacter, Escherichia, and Klebsiella are suitable to prepare the fermentation waste stream of the disclosed invention. Preferred biomass materials of this disclosure to serve as feedstock to generate the fermentation waste streams include glycerol-containing bio-diesel refinery waste, carbohydrate-rich brewery waste or any biomass-derived material containing one, two or more of the following carbohydrates: glucose, fructose, mannose, xylose, arabinose, galactose, mannitol, rhamnose, cellobiose, maltose and/or sucrose.

Microorganisms of the Enterobacter genus of this disclosure suitable for generation of the fermentation waste streams as feedstock for the green microalgae Chlorella protothecoides are preferentially (but not exclusively) Enterobacter sp. SGT-T4 and Enterobacter sp. SGT06-1. The microalgae and process described herein are suitable for conversion of metabolic waste products-containing bacterial fermentation wastes into oils or other microalgal value products for industrial scale production of oil or value product derivatives. Proposed industrial scale microalgae oil-producing systems will operate with fermentation wastes generated by the Enterobacter sp. SGT-T4™, Enterobacter sp. SGT06-1™, or derivatives of these microorganisms, at sites with traditionally large starch, cellulosics, hemicellulosics, and glycerol-containing waste loads, such as food processing industries, breweries, and bio-diesel refineries. The microalgae, waste feedstock processing procedure and cultivation methods of the disclosure can be effectively used for on-site, decentralized industrial scale production of bio-fuels in form of bio-diesel or bio-hydrogen. Therefore, the disclosed invention is expected to make significant contributions to food price stability, domestic energy security, natural resource conservation, land use protection and pollution prevention.

Microorganisms suitable to use the generated fermentation waste streams as feedstock for growth, oil- and other value products generation includes microorganisms belonging to the algal division of green algae (chlorophyta). Green algae are highly adaptable life forms which can grow under photoautotrophic as well as heterotrophic conditions. A preferred green algae of this disclosure is an algae which can build high biomass not only in the presence of light and carbon dioxide (CO₂) as sources of energy and carbon, but also can thrive under chemoheterotrophic cultivation conditions, i.e. in the presence of external organic compounds as carbon- and energy sources. Green microalgae, preferentially but not exclusively a species belonging to the Chlorella genus, e.g. Chlorella protothecoides, fulfill these criteria. Chlorella protothecoides not only grows well in light with only carbon dioxide as carbon source, but also has been reported to produce biomass and to generate high amounts of microalgal oils when cultivated with glucose as carbon source. In this disclosure, Chlorella protothecoides is used as a non-limiting example and shown to grow to high biomass and to generate high amounts of microalagal oil when cultivated under heterotrophic conditions using processed bacterial fermentation wastes as carbon feedstock. Microorganisms suitable for generation of the bacterial fermentation wastes of this disclosure belong to the bacterial family of enterobacteriaceae, and includes preferentially but not exclusively the enterobacteria species Enterobacter sp. SGT-T4 and Enterobacter sp. SGT06-1.

Microalgal Cultivation and Oil Production

The disclosure includes a method of culturing a green microalgae with a suitable algal culture medium and defined volumes of processed bacterial fermentation waste, generated by microorganisms such as Enterobacter sp. SGT-T4, Enterobacter sp. SGT06-1, or other members of the enterobacteriaceae family, to reach high biomass and to produce microalgal oil. Processed bacterial fermentation waste is added to the algal culture medium at a concentration chosen from a concentration between 20% (v/v) and 75% (v/v). The method may also comprise cultivation conditions that are suitable or advantageous for microalgal oil production, such as the use of an algal culture medium and/or conditions as described herein.

The disclosure thus includes an algal culture comprising a microalga of the disclosure and a culture medium or formulation as described herein. In some embodiments, the medium or formulation includes the combination of a source of one, two or more alkali phosphates, one or more inorganic salts, a processed protein extract, one or more nitrogen-containing compounds, biotin, nicotinic acid, pantothenic acid, and bacterial fermentation waste as feedstock, each of which is as described herein. The algal culture may be maintained or propagated under conditions that include a combination of a gaseous phase or formulation above the medium or directly bubbled into the medium, a suitable temperature, suitable agitation of the medium or formulation, suitable osmolarity, suitable salt concentration and an acceptable pH, each as described herein. In some cases, the gaseous phase comprises sterile-filtered air, carbon dioxide, or an inert or noble gas, which is optionally bubbled through a liquid medium or formulation. Non-limiting examples of a suitable temperature for the microalgae bio-reactor include at or below about 30° C. or about 28° C., about 26° C., about 24° C., about 22° C., or about 20° C. Non-limiting examples for the agitation of the algae-containing culture medium or formulation include induced convection caused by gas flow into the medium or by integrating a stirring device into the bio-reactor. Non-limiting examples of acceptable pH values for the algal culture medium include a pH at or below about 5.5, a pH at or below about 6.0, a pH at or below about 6.5, a pH at or below about 7.0, or a pH at or below about 7.5.

The disclosure also includes a method of extracting microalgal oil from the microalgae Chlorella protothecoides after cultivation in the presence of defined percentages of bacterial fermentation waste as carbon feedstock. The latter method includes pretreatment of the microalgal cell wall with defined amounts of a bio-catalyst, preferentially but not exclusively an exo- or endoglucanase (cellulase) enzyme, for a defined time, mechanical disintegration of the microalgal cell wall in the presence of dry ice and defined amounts of silicaceous materials, preferentially but not exclusively diatomaceous earth or Celite.

Having now generally described the invention, the same will be more readily understood through reference to the following examples which are provided by way of illustration, and are not intended to be limiting of the disclosed invention, unless specified.

### EXAMPLES

### Example 1

### General

### Environmental Sampling

The SGT-T4™ microorganism of the disclosure was isolated from the dissected gut of a termite species found in the U.S.A. For isolation, the gut of the surface-sterilized and dissected termite was carefully removed under sterile conditions, minced and transferred into sterile basic growth medium (6 g Tryptone, 3 g yeast extract, 10 g glucose, 0.3 g MgSO₄, 0.02 g CaCl₂, 67 mM K₂HPO₄/NaH₂PO₄ buffer, pH 7.0 in 1 liter distilled water). Serial solutions of the dissolved gut homogenates were made in the basic growth media and then incubated under aerobic and anaerobic conditions at 30° C. for several days. Aliquots of test tubes showing bacterial growth were streaked onto the surface of selective agar plates containing growth media and incubated at 35° C. for one to three days in a humified incubator.

### Growth Medium, Isolation and Cultivation

Single colonies of the plates grown under aerobic and anaerobic conditions were picked, re-inoculated in basic growth medium and restreaked on agar plates. Single colonies of these plates were picked and tested for hydrogen production as described below.

### Example 2

### Measurement of Gas and Hydrogen Production

Picked individual colonies were tested for total gas production using inverted Durham test tubes filled with either basic growth medium (10 ml) as described above or filled with other complex media (10 ml) as described in more detail in the examples below. Alternatively, total gas production of isolated colonies was detected using the BBL enterotube testing system. During this screening effort a microorganism, termed SGT-T4™, was discovered as a bacterium with the highest total gas production within a time period of less than 24 hours.

Hydrogen gas production of the microorganism was measured and achieved using following experimental set-up and incubation conditions. An aliquot (350 μl) of an over night culture of SGT-T4™ (grown in modified basic growth medium as described above) was inoculated into 50 ml of sterile complex medium 1 (14 g K₂HPO₄, 6 g KH₂PO₄, 5 g peptone, 2 g (NH₄)₂SO₄, 0.2 g MgSO₄×7H₂O, 1 ml trace element solution, 15 g glucose (or other carbon feedstock), pH 7.0 in 1 liter distilled water) and transferred into a 250 ml size fermentation vessel. After sealing off the fermentation vessel with a two-way inlet rubber stopper, the content of the vessel was flushed for 10 minutes with pure argon gas at a flow rate of 10 ml/min. The incubation vessel with the inoculated bacteria was placed in a shaking water bath or stirred fermentation platform and incubated at 37° C. Time-dependent generation of hydrogen gas in the vessel was monitored with the help of a linked fuel cell system (Hydro-Genius™, HelioCentris, Berlin, Germany).

The hydrogen gas-induced increase in current and voltage at the fuel cell was recorded with the help of a fuel cell-connected amperemeter (DT 830B multimeter) and voltmeter (Fluke 10 multimeter). Alternatively, the time-dependent evolution of hydrogen gas of the inoculated bacteria was measured by a liquid-gas exchange method using an upside-down graduated measuring cylinder (250 ml) which was tube-connected with the incubation vessel. The liquid in the cylinder was a freshly prepared 15% NaOH solution which quantitatively (>97%) absorbed the CO₂ fraction of the evolved gas from the fermentation vessel. Therefore, the gas production rate measured by the graduated measuring cylinder was considered as the hydrogen gas (H₂) production and was standardized to ml H₂ evolved per hour per liter fermentation volume. Using both hydrogen production monitoring and measurement methods, the isolated microorganisms SGT-T4™ was found to be a rapid and high hydrogen gas producer. The discovered hydrogen producing microorganism SGT-T4™ was further characterized and its cultivation conditions optimized for maximum and long term hydrogen production under batch conditions.

### Example 3

### Biochemical Analysis and Identification

An isolated colony of SGT-T4™ was inoculated into basic growth medium and grown at 37° C. between 12-24 hours. Morphological examinations and cell counting were performed with a compound light microscope (Olympus, Japan) using the oil immersion method. Gram-staining, which was performed by the Hucker method, and motility testing using the semisoft agar medium method revealed that the high hydrogen gas producing microorganism is a gram-negative, motile, non-sporulating and non-capsulated short rods which grow under aerobic and anaerobic growth conditions (see ‘General Properties’ in Table 1).

TABLE 1
GENERAL PROPERTIES OF SGT-T4

| | | Growth Ability | |
|---|---|---|---|
| Strain | SGT-T4 | Aerobic | Yes |
| Gram Staining | Negative | Anaerobic | Yes |
| Shape | short rod | Tryptone-Yeast Water | Yes* |
| Motility | Motile | Tryptone-Yeast Water | Yes* |
| | | (+Glucose) | |
| Spore | Non-spore | Minimum Medium | No* |
| Capsule | No | Minimum Medium | Yes* |
| | | (+Glucose) | |

*= under aerobic growth conditions; 1% glucose as carbon source

Based upon further examined biochemical properties of the isolated microorganism using the BBL Enterotube II system (BD Diagnostics, U.S.A.) and individual biochemical tests (see Table 2), the biochemical profile of SGT-T4™ appeared most similar to the reported features of the enterobacterium Enterobacter aerogenes (see Bergey's Manual of Determinative Bacteriology; 9th edition).

TABLE 2
BIOCHEMICAL PROPERTIES OF SGT-T4

| | SGT-T4 ™ | SGT06-1 ™ | E.a.* |
|---|---|---|---|
| Strain | | | |
| Gram stain | − | − | − |
| Spore stain | − | − | − |
| Motility | + | + | + |
| Indole | − | − | − |
| Voges-Proskauer (Acetoin) | + | + | + |
| Methyl Red | − | − | − |
| Citrate | + | + | + |
| Gas (f. Glucose) | + | + | + |
| H₂S | − | − | − |
| Urease | + | − | − |
| Phenylalanine deaminase | − | − | − |
| Lysine decarboxylase | + | − | + |
| Ornithine decarboxylase | + | + | + |
| Oxidase | − | − | − |
| Catalase | + | + | + |
| Growth &Acid (aerobic)#: | | | |
| D-Glucose (plus gas) | + | + | + |
| D-Adonitol | + | + | + |
| L-Arabinose (plus gas) | + | + | + |
| Cellobiose (plus gas) | + | + | + |
| Dulcitol | − | − | − |
| Lactose | − | − | + |
| Sucrose (plus gas) | + | + | + |
| Maltose (plus gas) | + | + | + |
| D-Xylose (plus gas) | + | + | + |
| D-Mannose (plus gas) | + | + | + |
| D-Sorbitol (plus gas) | + | + | + |
| D-Mannitol (plus gas) | + | + | + |
| L-Rhamnose (plus gas) | + | + | + |
| D-Galactose (plus gas) | + | + | n/a |
| Glycerol | + | − | n/a |
| Starch | + | + | n/a |
| Cellulose | − | − | n/a |

*E.a. = Enterobacter aerogenes; information based on Bergey's Manual of Determinative Bacteriology’ (9th edition)
n/a = data not available in Bergey's Manual of Determinative Bacteriology
#concentration for all carbohydrates under investigation = 1.5%; results observed and recorded after 24 hours incubation time

Despite the similarities to the enterobacterium Enterobacter aerogenes, SGT-T4™ showed three major differences to the reported characteristics of Enterobacter aerogenes. First it did not grow well with lactose as feedstock and did not generate significant amounts of gas after 24 hours incubation in the growth media. Second, in variation to known Enterobacter aerogenes bacteria, SGT-T4™ was able to grow and to evolve gas with urea as sole nitrogen source in the medium. Without being bound by theory, and offered to improve the understanding of the disclosure, this is consistent with the bacterium being urease-positive. Third, SGT-T4™ shows minimum spontaneous sedimentation in growth media after prolonged non-agitated incubation when directly compared with the commercially available bacterium Enterobacter aerogenes ATCC13048.

When directly compared with the biochemical properties of Enterobacter sp. SGT06-1™ (another hydrogen producing microorganism), SGT-T4™ showed three significant differences. First, SGT-T4™ but not SGT06-1™ was able to grow in pure glycerol as carbon feedstock and to generate gas. Second, in variation to the SGT06-1™ bacteria, SGT-T4™ was able to grow and to evolve gas with urea as sole nitrogen source in the medium. Finally, SGT-T4™, but not SGT06-1™, is a lysine decarboxylase enzyme-positive microorganism.

### Example 4

### Total Gas Production of SGT-T4 with Glucose and Zeolite Effect

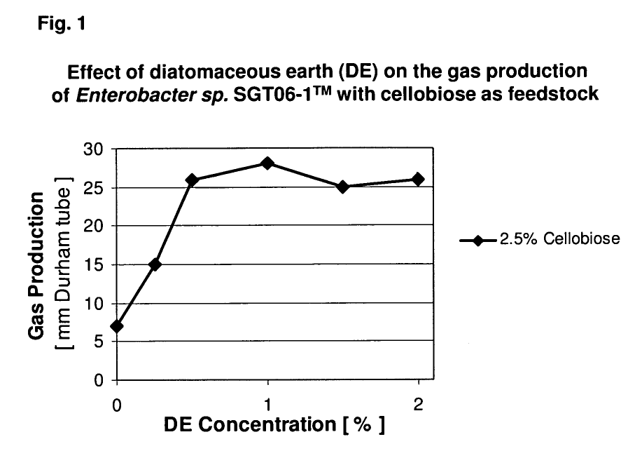

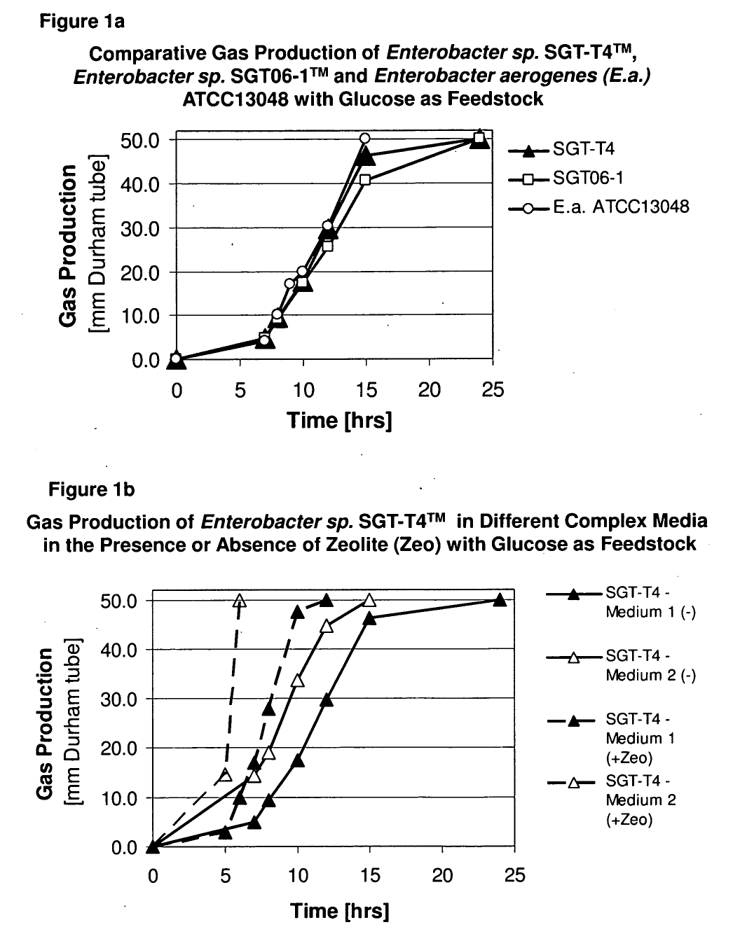

The high evolution of gas from SGT-T4™, especially under anaerobic conditions, as indicated by the BBL Enterotube analysis system was further analyzed. Direct comparisons were made of the total gas production from SGT-T4™ with Enterobacter sp. SGT06-1™ and Enterobacter aerogenes ATCC 13048 (E.a.), both known high hydrogen gas producing bacteria. Each of the three microorganisms was inoculated in Durham test tubes filled with 10 ml of peptone-glucose (PG) medium (14 g K₂HPO₄; 6 g KH₂PO₄; 5 g peptone; 2 g (NH₄)₂SO₄; 0.2 g MgSO₄×2H₂O; 15 g glucose per 1 liter). The bacteria were incubated at 37° C. in an incubator and monitored for the evolution of gas at defined time intervals over 24 hours. The results of this set of experiments, which are shown in FIG. 1a (plotted as mm total gas accumulation in the inverted Durham tubes) confirm the high gas production from SGT-T4™. The total gas production of SGT-T4™ with glucose as carbon feedstock showed no significant difference to that of SGT06-1™ or of Enterobacter aerogenes ATCC 13048. Similar high gas production under anaerobic conditions was observed by incubating the newly isolated microorganism in Durham test tubes filled with glucose minimum (synthetic) medium (results not shown).

Next a series of experiments was conducted to find conditions which further increase the high hydrogen production rate of the isolated microorganism. For this, SGT-T4™ was inoculated in Durham test tubes filled with 10 ml of either peptone-glucose (PG) medium (14 g K₂HPO₄; 6 g KH₂PO₄; 5 g peptone; 2 g (NH₄)₂SO₄; 0.2 g MgSO₄×7H₂O; 21 mg CaCl₂×2H₂O; 20 g glucose per 1 liter) or tryptone-yeast-glucose (TYG) medium (7 g K₂HPO₄; 5.5 g KH₂PO₄; 5 g tryptone; 5 g yeast extract; 1 g (NH₄)₂SO₄; 0.25 g MgSO₄×7H₂O; 0.12 g Na₂MoO₄×2H₂O; 2 mg nicotinic acid; 0.172 mg Na₂SeO₃; 0.02 mg NiCl₂; 2 mg MnCl₂×4H₂O; 20 g glucose per 1 liter) in the presence or absence of 2.5% of the strongly absorbent aluminosilicate zeolite (Zeo). The bacteria were incubated at 37° C. in an incubator and monitored for the evolution of gas at defined time intervals over 24 hours. The results of this set of experiments, which are shown in FIG. 1b, show that the high gas production of SGT-T4™ in PG medium is further increased in TYG medium and that the gas production in both media is significantly increased in the presence of 2.5% zeolite in the growth medium.

The calculated total gas production rate of SGT-T4™ was about 43 percent (43%) higher when it is cultivated in TYG medium (261 ml gas/hour per liter in PG medium versus 374 ml gas/hour per liter in TYG medium). Presence of zeolite in the growth medium increased the gas production in PG medium by a factor of 1.87 (87%) and in TYG medium to more than 8.6 times (865%). The effect of zeolite on the gas production of SGT-T4 in TYG medium is dramatic and is the highest percent gas production rate increase ever reported in the published literature and known to the present inventors.

### Example 5

### Hydrogen Production of SGT-T4 with Glucose and Zeolite Effect

The hydrogen production rate of SGT-T4™ with glucose as carbon feedstock was measured in the presence or absence of 2.5% zeolite in the medium. For this a liquid-gas exchange method was used which consisted of an upside-down graduated cylinder filled with a 15% NaOH solution that was tube-connected with the gas outlet of the bio-reactor containing the cultivated bacterium under investigation.

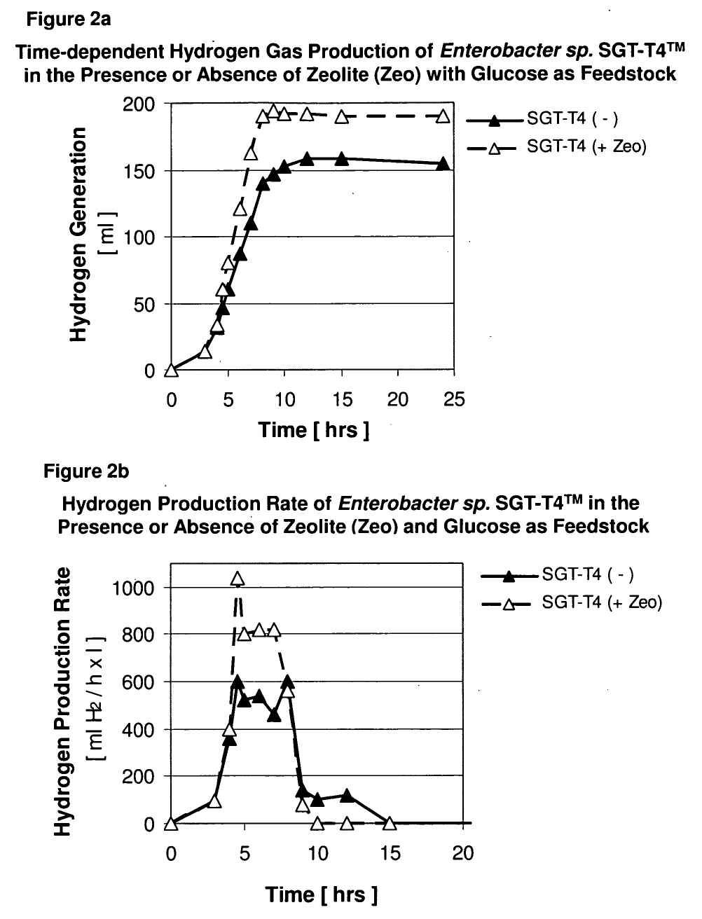

Due to the absorption of carbon dioxide—the only concomitantly released gas by the isolated bacterium SGT-T4™—by the NaOH solution in the inverted cylinder, the gas production rate measured with the help of a graduated cylinder was considered to be the hydrogen evolution rate of the bacteria under investigation. Using this method and incubating the bacteria under batch conditions in 50 nm tryptone-yeast medium at a temperature of 37° C. and with 2% glucose as feedstock, SGT-T4 evolved 154 ml of hydrogen gas (H₂) in 24 hours (FIG. 2a) and the maximum hydrogen production rate of SGT-T4™ was measured to be 600 ml hydrogen gas (H₂) produced per hour per liter (ml/h×l) (FIG. 2b). This rate is the highest hydrogen production rate under batch conditions ever reported for a hydrogen producing microbe with glucose as feedstock (for comparison see Table 3 below) and exceeds the high total gas and hydrogen production rates reported by Taguchi et al. (U.S. Pat. No. 5,350,692) for the anaerobic microorganisms AM21B and AM37 in peptone-yeast glucose (PYG) medium. The high hydrogen production rate of SGT-T4 with glucose as feedstock was increased about 77 percent (77%) in the presence of 2.5% zeolite in the growth medium. At about 4.5 hours incubation time a hydrogen production rate of more than 1 liter of H₂ per hour per liter (1,060 ml H₂/h×l) was measured (FIG. 2b). The very high hydrogen gas production rate of the microorganism SGT-T4™ under the chosen incubation conditions was further confirmed by detecting the generated hydrogen gas with the help of a vessel-connected and calibrated fuel cell system (Hydro-Genius™, HeliCentris, Berlin, Germany) (Data not shown).

TABLE 3
COMPARATIVE HYDROGEN PRODUCTION RATES

| | Hydrogen Gas Rate | |
|---|---|---|
| Species* | (ml H₂/h × l) | References |
| Enterobacter sp. SGT-T4 ™ | 600 | Schmid E. et al. |
| | 1,060⁺ | (unpublished results) |
| Enterobacter sp. SGT06-1 ™ | 460⁺ | Schmid E. et al. |
| | | (unpublished results) |
| Klebsiella oxytoca | 87.5 | Minnan L. et al., Res. Microbiol. |
| | | 156(1): 76-81 (2005) |
| Citrobacter freundii | 90 | Kumar G. R. et al., Indian J. Exp. |
| | | Biol. 27(9): 824-825 (1989) |
| Enterobacter aerogenes | 253 | Tanisho S. et al., J. Chem. Eng. |
| E.82005 | | (Japan) 16: 529ff (1983); |
| | | Tanisho S. et al., Int. J. Hydrogen |
| | | Energy 12: 623ff (1987) |
| Enterobacter aerogenes | 372 | Ito T., et al., J. Biosci. Bioeng. 97(4): |
| HU-101 | | 227-232 (2004) |
| Enterobacter aerogenes | 120 | Yokoi H. et al., J. Ferment. Bioeng. |
| | | 80: 571ff (1995) |
| Clostridium beijerinckii | 210 | Taguchi F. et al., United States |
| | | Patent #5,350,692 (Sept. 27, 1994) |
| Clostridium butyricum | 75 | Ogino H. et al., Biotechnol. Prog. |
| | | 21(6): 1786-1788 (2005) |
| Mixed Anaerobes | 230 | Iyer P. et al., Appl. Microbiol. |
| | | Biotechnol. 66: 166-173 (2004) |
| Mixed bacterial cultures | 74.7 | Van Ginkel S. et al., Environ. Sci. |
| | | Technol. 35(24): 4726-4730 (2001) |
| Thermotoga elfii | 125 | Van Niel E. W. J. et al., Hydrogen |
| | | Energy 27: 1391-1398 (2002) |
| Caldicellulosiruptor- | 250 | Van Niel E. W. J. et al., Hydrogen |
| saccharolyticus | | Energy 27: 1391-1398 (2002) |
| Caldicellulosiruptor- | 250 | Kadar Z. et al., Appl. Biochem. |
| saccharolyticus | | Biotechnol. 113-116: 497-508 (2004) |
| Thermotoga neapolitana | 460 | Van Ooteghem S. A. et al., Appl. |
| | | Biochem. Biotechnol. 98-100: 177-189 |
| | | (2002) |

*all microorganisms cultivated in batch cultures in the presence of glucose
⁺cultivated in the presence of 2.5% natural zeolite (clinoptilolite) in the medium

SGT-T4™ shows rapid growth and reaches high optical densities not only in the presence of the carbohydrate glucose, but also when cultivated in the presence of other carbohydrate feedstock, such as sucrose, cellobiose, maltose, xylose, arabinose, rhamnose, galactose, sorbitol, mannitol, and mannose (data not shown).

### Example 6

### Total Gas Production of SGT-T4™ with Different Carbon Feedstock

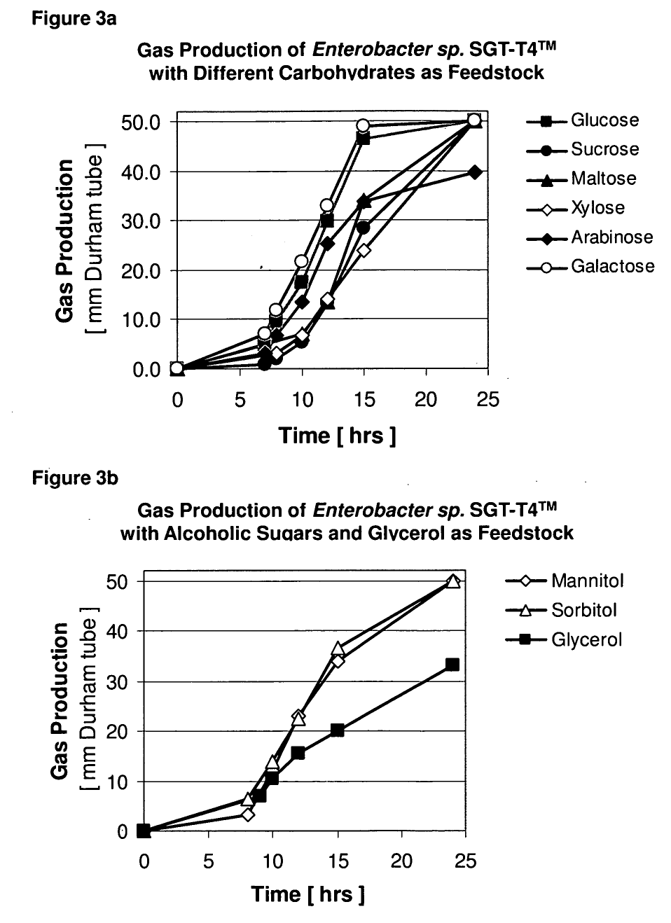

The gas production capacity of SGT-T4™ was tested in the presence of carbohydrates and carbon feedstock other than glucose. This tested whether SGT-T4™ is metabolically versatile and is capable of generating comparatively high amounts of gas in the presence of important biomass-derived carbon compounds as feedstock. The present inventors were especially interested whether the disaccharides sucrose and maltose, the hemicellulosics-derived carbohydrates xylose, arabinose and galactose, the alcoholic sugars mannitol and sorbitol, as well as the phospholipid and fat-derived carbon compound glycerol also serve as suitable feedstock for the isolated microorganism. As shown in FIG. 3a, SGT-T4™ generates high amounts of gas with glucose as feedstock (grey squares), and also when cultured in the presence of maltose, sucrose, arabinose, xylose and galactose. It is of interest that the time-dependent gas production of SGT-T4™ shows a distinctive prolonged lag phase with maltose, sucrose, xylose and arabinose as feedstock when directly compared with glucose, while SGT-T4 responded with an even stronger gas production than with glucose in the presence of the monosaccharide galactose as carbon source. This finding, where the isolated bacterium SGT-T4™ is able to generate high amounts of gas from more than monosaccharide glucose as feedstock, but also from the important plant-derived disaccharides sucrose and maltose, as well as in the presence of key hemicellulosics sugars, such as xylose and arabinose, is of high commercial value. It allows simplified and cost saving future industrial scale hydrogen production from traditionally high sucrose-containing wastes, such as bagasse and food industry wastes, maltose-containing waste streams, such as brewery wastes, and from materials abundant in hemicellulose, such as plant matter.

### Example 7

### Gas Production from Alcoholic Sugars and Glycerol

Yet another important set of studies conducted was the capability of SGT-T4™ to generate high quantities of gas in the presence of the alcoholic sugars mannitol and sorbitol, and when cultured in the presence of the tertiary alcohol glycerol. As shown in FIG. 2b, SGT-T4™ generates very high amounts of gas in the presence of the alcoholic sugars mannitol and sorbitol in the growth medium within 24 hours incubation time. The gas production of SGT-T4™ with glycerol as carbon feedstock was not as high as with mannitol or sorbitol under the chosen incubation conditions. The observation whereas SGT-T4™ is capable of generating high amounts of hydrogen gas from the alcoholic sugars mannitol and sorbitol, makes it a potentially attractive microorganism for future industrial scale generation of hydrogen energy from sources and waste streams rich in these alcoholic sugars, such as brown algae and nutritional industry.

### Example 8

### Increased Gas Production of SGT-T4 with Glycerol or Crude Bio-Diesel Production Waste in the Presence of Zeolite

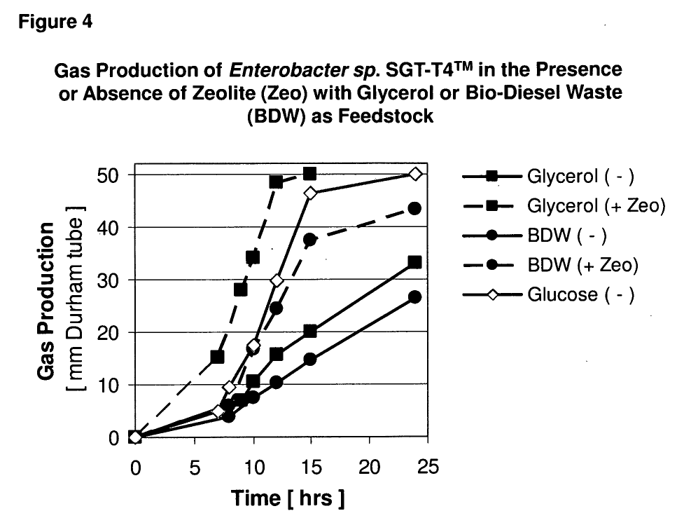

A set of studies was conducted to study the effect of an aluminosilicate mineral on the gas production of SGT-T4™ when cultured in the presence of the tertiary alcohol glycerol. As shown in FIG. 4, the low gas production rate of SGT-T4™ with glycerol (300 mM) and without zeolite in the growth medium (91 ml gas per hour per liter) was increased 2.4 times when defined amounts of zeolite (2.5%) were present in the growth medium during the incubations. In the presence of zeolite the gas production rate of SGT-T4™ increased to about 220 ml gas per hour per liter. Since bio-diesel waste contain high (>40%) concentrations of glycerol, the authors of this disclosure conducted experiments to test whether SGT-T4™ is capable to generate high amounts of gas when cultivated in the presence of a defined volume of glycerol-containing crude bio-diesel waste (BDW) collected from a local bio-diesel processor. For this SGT-T4™ was incubated in 10 ml tryptone-yeast growth medium containing 0.75 ml of crude BDW in the presence or absence of defined amounts of zeolite. As shown in FIG. 4, the low total gas production rate of SGT-T4™ with bio-diesel waste (BDW) and without zeolite in the growth medium of about 53 ml gas per hour per liter) was increased more than 2.7 times when defined amounts of zeolite (2.5%) were present in the growth medium during the incubations. In the presence of zeolite the gas production rate of SGT-T4™ increased to about 148 ml gas per hour per liter, which was almost as high as the gas evolution rate observed with glucose as carbon feedstock (173 ml gas per hour per liter; in the absence of zeolite). The observation that SGT-T4™ is capable of generating high amounts of gas from the tertiary alcohol glycerol and also from high glycerol-containing bio-diesel waste in the presence of zeolite mineral in the growth medium makes it a potentially attractive microorganism for industrial scale hydrogen production for the rapidly developing bio-diesel processing industry.

### Example 9

### Increased Hydrogen Gas Production of SGT-T4 with Glycerol or Crude Bio-Diesel Production Waste in the Presence of Zeolite

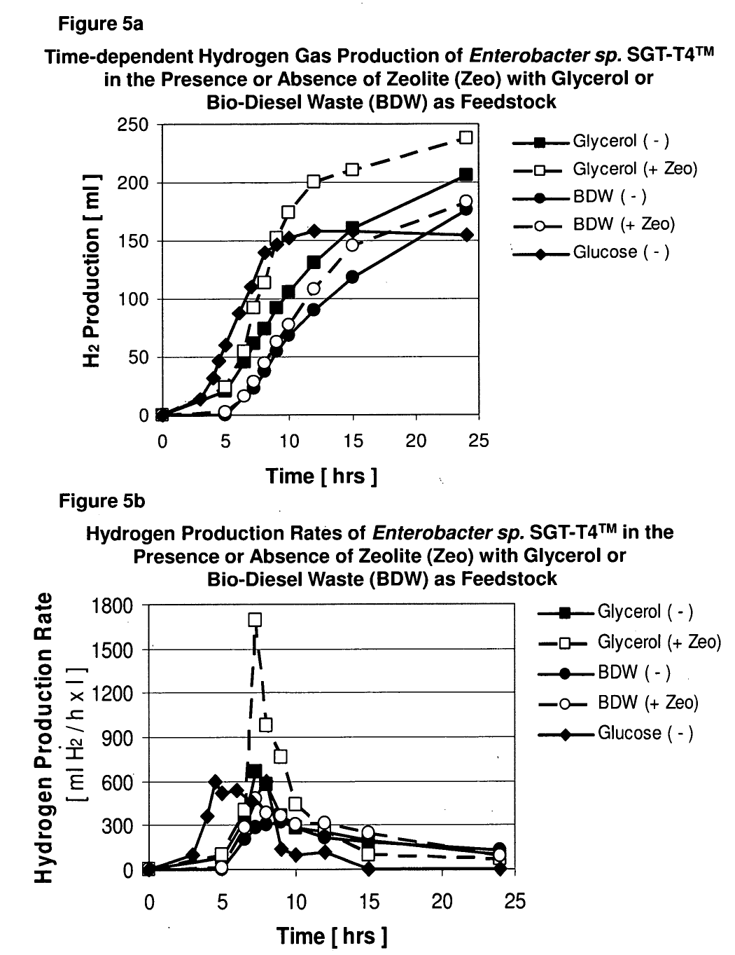

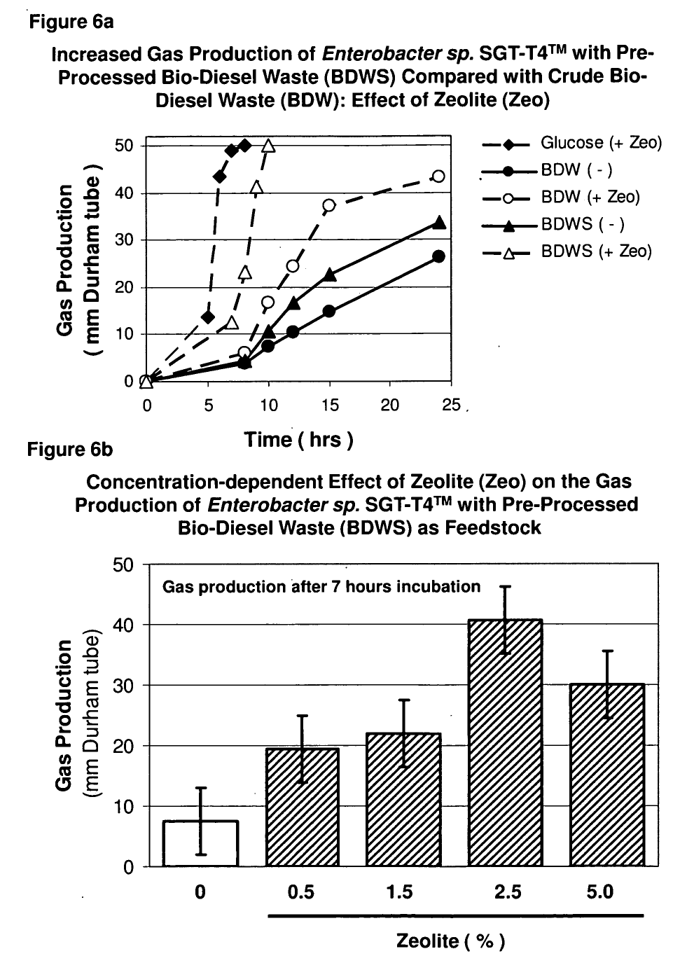

The amount of hydrogen gas evolved over time and the hydrogen production rate of SGT-T4™ with glycerol or crude bio-diesel waste (BDW) as carbon feedstock was examined in the presence or absence of zeolite in the growth medium. For this, the same liquid-gas exchange method was used as described in more detail in Example 5. It consisted of an upside-down graduated cylinder filled with a 15% NaOH solution that was tube-connected with one of the outlets of the bio-reactor. The bio-reactor was filled with 50 ml of growth medium containing the cultivated bacterium under investigation and where indicated in FIG. 5 with 2.5% zeolite material. Due to the absorption of carbon dioxide—the only concomitantly released gas by the isolated bacterium SGT-T4™—by the NaOH solution in the inverted graduated cylinder, the gas production rate measured with the use of a graduated cylinder was considered to be the hydrogen evolution rate of the bacteria under investigation. As shown in FIG. 5a, using this method and incubating the bacteria under batch conditions in 50 ml tryptone-yeast medium at a temperature of 37° C. and with pure industrial glycerol as feedstock, SGT-T4 evolved 206 ml of hydrogen gas (H₂) in 24 hours in the absence of zeolite in the growth medium. The volume of hydrogen gas evolved by SGT-T4™ after 24 hour incubation increased more than 15% to 237 ml in the presence of 2.5% zeolite in the growth medium. The calculated maximum hydrogen production rate of SGT-T4™ with glycerol as feedstock occurred at around 7 hours incubation time and was 667 ml hydrogen gas (H₂) produced per hour per liter (667 ml/h×l) in the absence of zeolite (FIG. 5b). This remarkably high rate further increased to 1,689 ml hydrogen gas (H₂) produced per hour per liter (1,689 ml/h×l) when 2.5% zeolite was present in the growth medium (FIG. 5b) accounting for a rate increase of more than 250%.

This high rate of SGT-T4™ with glycerol as feedstock and in the presence of defined amounts of zeolite in the growth medium is the highest hydrogen production rate under batch conditions ever reported for a hydrogen producing microbe with this feedstock. For comparison, the volumes of hydrogen gas evolved by SGT-T4™ from 300 mM glycerol in the presence (237 ml; 209 mM) or absence (206 ml; 182 mM) of zeolite within 24 hours both exceed the reported H₂ volume of 93 ml (53 mM) generated by Enterobacter aerogenes HU-101 in 24 hours in the presence of 110 mM glycerol as feedstock [Ito T., et al.; J. Bioscience & Bioengineering 100(3): 260-265 (2005)]. A 300 ml culture of Enterobacter aerogenes NBRC12010 was recently reported to generate about 265 ml (40 mM) H₂ from glycerol (110 mM) in 24 hours under chemostat conditions (see Sakai S. & Yagashita T.; Biotechnology & Bioengineering 98(2): 340-348 (2007)). Based on these comparisons, the present inventors believe that SGT-T4™ is an ideal candidate microorganism for conversion of glycerol and glycerol-containing waste streams into clean hydrogen energy under favorably high production rate. To test this, SGT-T4™ was incubated in 50 ml tryptone-yeast growth media together with 3.8 ml of collected bio-diesel waste in the presence or absence of zeolite.

As shown in FIG. 5a, using the earlier described liquid-gas exchange method and incubating SGT-T4™ under batch conditions in 50 ml tryptone-yeast medium at a temperature of 37° C. and with crude bio-diesel waste (BDW) as feedstock, SGT-T4 evolved 176 ml of hydrogen gas (H₂) in 24 hours in the absence of zeolite in the growth medium. The volume of hydrogen gas evolved by SGT-T4™ after 24 hour incubation increased to 183 ml in the presence of 2.5% zeolite in the growth medium. The calculated maximum hydrogen production rate of SGT-T4™ with BDW as feedstock occurred between 7 and 8 hours incubation time and was 320 ml hydrogen gas (H₂) produced per hour per liter (320 ml/h×l) in the absence of zeolite (FIG. 5b). This rate further increased to almost 500 ml hydrogen gas (H₂) produced per hour per liter (480 ml/h×l) when 2.5% zeolite was present in the growth medium (FIG. 5b) accounting to a rate increase of about 50%.

### Example 10

### Increased Hydrogen Gas Production of SGT-T4 with Pre-Processed Bio-Diesel Waste in the Presence of Zeolite

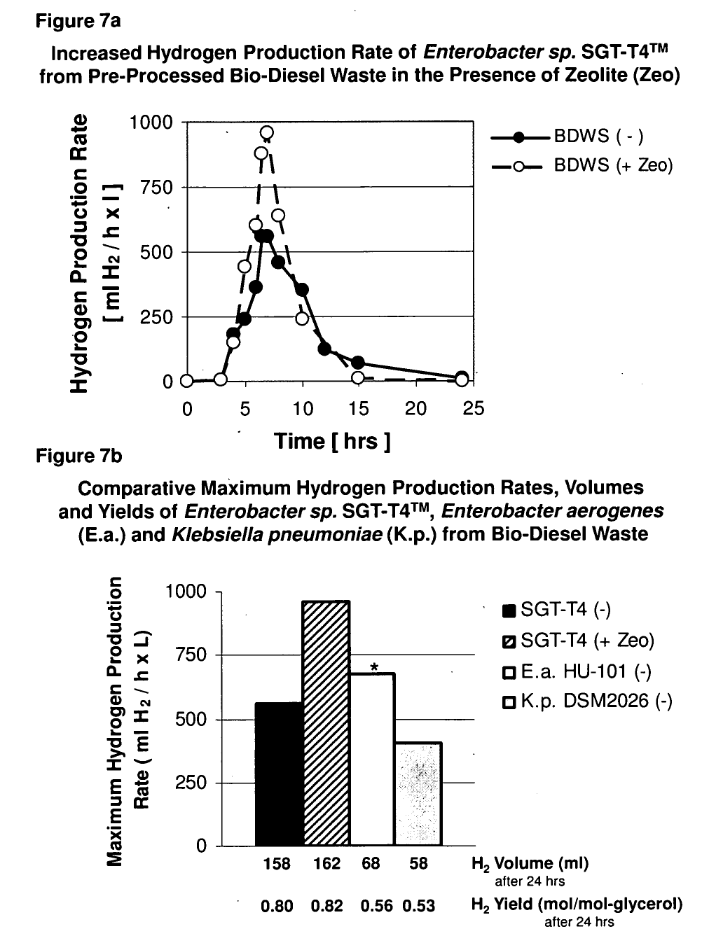

The amount of hydrogen gas evolved over time, hydrogen production rate and hydrogen production yield of SGT-T4™ with pre-processed bio-diesel waste (BDWS) as carbon feedstock was examined in the presence or absence of zeolite in the growth medium. Pre-processed bio-diesel waste solution (BDWS) was freshly prepared before the experiment by dissolving 40 ml of pre-cleared bio-diesel waste (BDW) in 460 ml of sterile distilled water followed by pH-neutralization of the alkaline pH of the BDWS with a 6N HCl solution. In this example, the same liquid-gas exchange method was used as described in more detail in Examples 5 and 9 to measure the generation of hydrogen gas by SGT-T4™. It consisted of an upside-down graduated cylinder filled with a 15% NaOH solution that was tube-connected with one of the outlets of the bio-reactor. The bio-reactor was filled with 25 ml of complex growth medium and 25 ml of BDWS containing the cultivated bacterium under investigation and where indicated in FIG. 7a with 2.5% zeolite material. Due to the absorption of carbon dioxide—the only concomitantly released gas by the isolated bacterium SGT-T4™—by the NaOH solution in the inverted cylinder, the gas production rate measured with the help of the graduated cylinder was considered to be the hydrogen evolution rate of the bacteria under investigation.

As shown in FIG. 7a, using this method and incubating the bacteria under batch conditions in 25 ml tryptone-yeast medium at a temperature of 37° C. and with 25 ml BDWS as feedstock, SGT-T4 evolved 158 ml of hydrogen gas (H₂) in 24 hours in the absence of zeolite in the growth medium. The calculated maximum hydrogen production rate of SGT-T4™ with BDWS as feedstock and in the absence of zeolite in the reaction vessel occurred at around 7 hours incubation time and was 560 ml hydrogen gas (H₂) produced per hour per liter (560 ml/h×l). Under identical incubation conditions, this high hydrogen production rate further increased to 960 ml hydrogen gas (H₂) produced per hour per liter (960 ml/h×l) when 2.5% zeolite was present in the growth medium accounting to a rate increase of more than 70%. The measured hydrogen production rate of SGT-T4™ with pre-processed bio-diesel waste solution (BDWS) as feedstock and in the presence of 2.5% zeolite in the growth medium is the highest hydrogen production rate under batch conditions reported for a hydrogen producing microbe with bio-diesel refinery waste as feedstock to date. For comparison, the hydrogen production rate (960 ml H₂/h×l), yield (0.82) and 24 hour volume of hydrogen gas (162 ml) evolved by SGT-T4™ from BDWS in the presence of zeolite exceeds the reported hydrogen production rates, yields and 24 hour volumes of the known hydrogen producing microbes Enterobacter aerogenes HU-101 (678 ml H₂/h×l, 0.56, 68 ml) (see Ito T., et al.; J. Bioscience & Bioengineering 100(3): 260-265 (2005)) and Klebsiella pneumoniae DSM2026 (402 ml H₂/h×l, 0.53, 58 ml) [Liu F. & Fang B.; Biotechnol. J. 2(3): 374-380 (2007)] from bio-diesel waste as feedstock (FIG. 7b). Based on these comparisons, the authors of this disclosure believe that SGT-T4™ is an ideal candidate microorganism for economical conversion of glycerol-containing waste streams, most prominently bio-diesel waste refinery waste, into clean hydrogen energy under favorably high production rate conditions.

### Example 11

### Comparison of the Sedimentation Behavior of SGT-T4™ with Other Enterobacteria

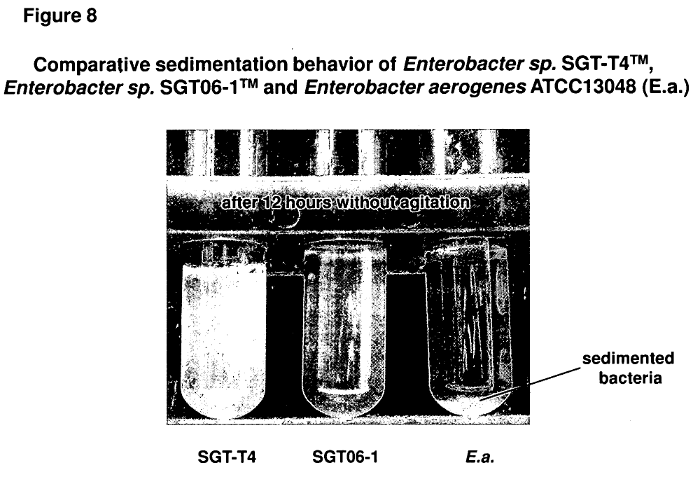

During comparative functional studies with the isolated microorganism SGT-T4™ and commercially available enterobacteria, such as the biochemically most closely related Enterobacter aerogenes species, the present inventors observed a strikingly different sedimentation behavior of SGT-T4™ in the growth media used in these studies. As shown in FIG. 8, when directly compared with Enterobacter aerogenes ATCC13048, SGT-T4™ showed no signs of sedimentation and no bacterial cell pellet formed at the bottom of the test tube after 12 hours incubation in the absence of test tube agitation during this incubation period. That significant difference between the sedimentation behavior of SGT-T4™ and a commercially available Enterobacter aerogenes species is indicative of significant differences in cell morphologies and/or motility. This low sedimentation behavior of SGT-T4™ might be beneficial in large scale bio-reactor environments where continuous stirring of the media is usually required which is a significant operation cost factor.

### Example 12

### PCR and 16S rRNA Gene Sequence Analysis

To better assign microorganism SGT-T4™ to the genus Enterobacter aerogenes within the enterobacteria family, molecular biological methods were used to identify the isolate by 16S-rRNA gene sequence analysis. For direct comparison and for serving as an internal control of the following procedure, PCR with DNA isolated from Enterobacter aerogenes (ATCC13048 strain) was used as an internal standard and control of the applied method (data not shown).

PCR-dependent 16S rRNA gene sequence analysis was carried out as follows. Isolates were grown in basic growth medium A for 20-24 hours at 37° C. and genomic DNA was isolated from pellets of collected bacterial cells (1 ml) using the Qiagen silica spin column method. A fragment of about 700 bp of the 16S rRNA gene of the isolated genomic DNA of SGT-T4™ was amplified by PCR using a designed “universal” 16S rRNA primer pair (SGT-UNI04fw3 and SGT-UNI04rv2 (see Table 4 below). SGT-UNI04fw3 and SGT-UNI04rv2 recognize highly conserved nucleotide sequences of the GenBank-deposited 16S rDNA sequence (nucleotide 140-160; nucleotide 824-841) of Citrobacter freundii ATCC 29935 (gi: 174064), and span a hypervariable region of the C. freundii 16S rRNA gene.

TABLE 4
USED PCR PRIMER FOR 16S rDNA ANALYSIS OF SGT-T4™

| | | |
|---|---|---|
| SGT-UNI04-fw3 | 5′- TGGAGGGGGATAACTACTGG -3′ | (SEQ ID |
| | | No:2) |
| SGT-UNI04-rv2 | 5′- GGCACAACCTCCAAGTCG -3′ | (SEQ ID |
| | | No:3) |

Twenty picomoles of forward primer (SGT-UNI04-fw3) and reverse primer (SGT-UNI04-rv2) were used in the PCR reaction. The PCR reaction mixture further contained 0.5 units Taq polymerase (Invitrogen), 500 ng of genomic DNA, 0.1 mmol/l of each nucleotide (dNTPs) and 1.5 mM MgCl₂, in a total volume of 201. A fragment of the 16S rRNA gene was amplified after 35 cycles in an automated thermal cycler (Mycycler, BioRad, Inc., CA) using following temperature profile: (4 min at 95° C.; (30 s at 95° C., 30 s at 53° C., 2 min at 72° C.)_{35×}; 5 min at 72° C.).

After separation by low melting agarose gel electrophoresis, the 16S-rRNA PCR product was excised and purified with use of the Qiagen gel purification kit. The base sequence of the purified 16S rRNA gene segment was determined by using the Tag Dye Deoxy Terminator Cycle Sequencing method (Seqxcel Inc., San Diego, Calif.) and compared with the nucleotide sequences deposited with the NCBI (National Center for Biological Information) database (all GenBank+EMBL+DDBJ+PDB sequences). A comparative analysis of the retrieved 671 base sequence (see Table 5) of SGT-T4™ was done with the GenBank database using NCBI BLAST (blastn & MegaBlast). It revealed that SGT-T4™ is related to gram-negative bacteria showing highest sequence similarity to members of the enterobacteriaceae family. The four top scoring sequence similarities reported for the submitted 16S rRNA gene sequences of the following databank-deposited microorganisms are listed below (rankings based on lowest Expect (E) values and highest maximum score):

1. Uncultured bacterium clone SJTU_B_14_72
(gi: 126113270; Accession #: EF402955.1)

| | | |
|---|---|---|
| Maximum Score: 756 | E Value = 0.0 | Max. Identity: 87% |

2. Enterobacter sp. DAP21
(gi: 163931346; Accession #: EU302846.1)

| | | |
|---|---|---|
| Maximum Score: 754 | E Value = 0.0 | Max. Identity: 87% |

3. Uncultured Enterobacteriaceae bacterium clone M7-54
(gi: 175941128; Accession #: EU530476.1)

| | | |
|---|---|---|
| Maximum Score: 752 | E Value = 0 | Max. Identity: 87% |

4. Uncultured Enterobacteriaceae bacterium clone M7-52
(gi: 175941126; Accession #: EU530474.1)

| | | |
|---|---|---|
| Maximum Score: 752 | E Value = 0.0 | Max. Identity: 87% |

Of the 100 reported most significant matches with the isolated 16S rDNA fragment, 27 out of 100 were enterobacteria species, 17/100 were uncultured enterobacteriaceae bacteria and 38/100 of the sequence homologies were reported for uncultured bacteria. Summarized, genomic DNA was isolated from SGT-T4™, and the base sequence has been successfully analyzed with an obtained 16S rDNA fragment. The microorganism SGT-T4™ is believed to belong to the enterobacteriaceae family based on this sequence analysis. Because the level of 16S rDNA gene identity with their closest taxonomically named relatives was less than 88%, and due to observed differences in urea utilization, lactose metabolism and motility between microorganism SGT-T4™ and Enterobacter aerogenes species, the isolated microorganism is believed to be an Enterobacter and perhaps represents a new species based on the presented unique biochemical and genetic features. Via the 16S rDNA gene analysis, which closely related the isolated microorganism to the enterobacterial species Enterobacter sp. DAP21, the microorganism is named Enterobacter sp. SGT-T4™ for further reference and preliminary classification. The isolated microorganism Enterobacter sp. SGT-T4™ was deposited with the American Type Tissue Collection (ATCC) on Apr. 10, 2008 with accession no. ATCC No: PTA-9150.

TABLE 5

BASE SEQUENCE OF 16S rRNA GENE FRAGMENT OF
SGT-T4™ (SEQ ID No:1)

CACATCGCAT ACGTCGCAGA CCAAAGTGGG GGACCTTCGG
GCCTCATGCC ATCAGATGTG CCCAGATGGG ATTAGCTAGT
AGGTGGGGTA ATGGCTCACC TAGGCGACGA TCCCTAGCTG
GTCTGAGAGG ATGACCAGCC ACACTGGAAC TGATACACGG
TCCAGACTCC TACGGGAGGC AGCAGTGGGG AATATTGATT
TATGGGCGCA AGCCTGATGC AGCCATGCCG CGTGTATGAA
CAAGGCCTTC CGATTGTAAA TTGCTTTCTC CGAATAGGAA
GGCCTGCTGG TTAATAACCT TGCGGATTGA CTTTACTCGC
AAACGAAGCA CCGGCTAACT CCGTGCCTTA AGCCCTTCCT
CCTCGGAGGG TGCACTTTTT AATCCGAATT ACTGGTTCTT
AAGCGCACGC TGGCTGCCTG TCGCTTGCGA TGTGAAATCC
CCGGGCTCCA CCTGGGAACT GCATTCGAAA CTGGACCGCT
AGAGTCTTGT AGAGGGGGGT GGAATTCCTC GTGTACCGGT
GAAATGCGTA CAGATCTGGA AGAATACCCC CCACCAAGGC
GGCCCCCTGG ACAAAGACTG ACTCTCAGGT GCAAAACCGT
GGGGAGCCCA CTTGATTATA TACCCTGGTA GTCCACTCCG
CTACCGATGT CAACTTGATT CCCCCCTCCA A (671 bp)

### Example 13

### Growth with Absorptive Material

Even though exemplars of disclosure utilize an enterobacterium, termed Enterobacter sp. SGT06-1™, the disclosed methods can be applied to any microorganism capable of generating hydrogen gas. To achieve the hydrogen production rate-enhancing effect with the disclosed absorptive materials, the hydrogen generating microorganism was incubated in a defined volume of a sterile basic growth medium containing (per liter) 14 g K₂HPO₄, 6 g KH₂PO₄, 5 g peptone, 2 g (NH₄)₂SO₄, 5-25 g of a carbon source, e.g. glucose, 0.2 g MgSO₄ with a pH of 7.0. The inoculated sample was incubated at a temperature of 37° C. in the presence of 10 grams (per liter) of an absorptive material, preferentially but not exclusively diatomaceous earth (DE) in a water bath (fuel cell experiments) or incubator (Durham tube experiments) with or without occasional stirring.

### Example 14

### Measurement of Gas and Hydrogen Production

Microorganisms used in these studies were tested for gas production in the absence or presence of absorptive materials using either inverted Durham test tubes or a rubber stoppered fuel cell-coupled fermentation vessel filled with basic growth medium as described above. For most total gas and hydrogen gas experiments the concentration of the carbohydrate feedstock, e.g. glucose or sucrose, was 2.5%. The total gas accumulated in the inverted Durham tubes was measured as mm change of trapped gas within the tubes at time intervals shown in the Figures. The effect of the absorptive materials on the hydrogen production rate of the microorganism was measured and achieved using the following experimental set-up and incubation conditions.

An overnight culture (0.35 ml) of SGT 06-1™ was inoculated into 50 ml of modified basic growth medium as described above and placed into a 250 ml vessel, e.g. Erlenmeyer flask (Pyrex quality). As indicated in the Figures of this disclosure, in some cases 500 mg of diatomaceous earth (or other absorptive materials) were added to the vessel to make a 1% final concentration of the absorptive material. After closing the incubation vessel with a two-way inlet rubber stopper, the content of the vessel was flushed with pure argon gas for 5 minutes at a flow rate of 10 ml/min. The incubation vessel with bacteria was placed in a water bath and incubated at 37° C. with gentle shaking every 2-3 hours. After 3 hours incubation, a constant argon flow of 10 ml/min was applied to the vessel using one of the inlets of the fermentation vessels. The time-dependent generation of hydrogen gas in the vessel was monitored with the help of an in-line linked fuel cell system (Hydro-Genius™, HelioCentris, Berlin, Germany). The hydrogen gas-induced increase in current and voltage at the fuel cell was recorded with the help of a fuel cell-connected amperemeter (DT 830B multimeter) and voltmeter (Fluke 10 multimeter). Under these conditions, the microorganism SGT 06-1™ was found to show much faster and higher hydrogen production with absorptive material present in the fermentation vessel.

### Example 15

### Increased Hydrogen Production Rate with Increasing Amounts of Diatomaceous Earth

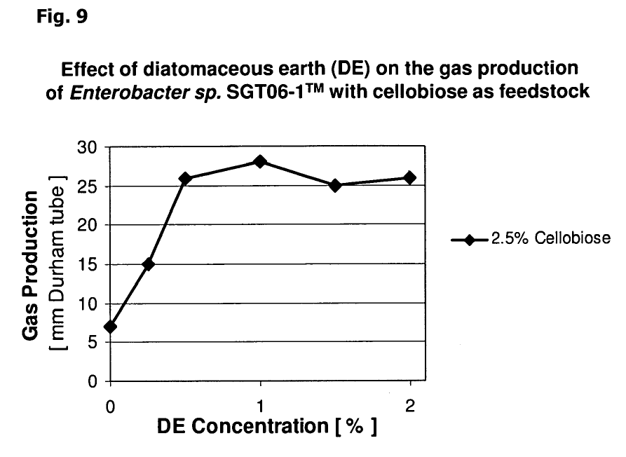

The effect of increasing amounts of the absorptive material diatomaceous earth (DE) on total gas production of the microorganism SGT 06-1™ was tested with 2.5% cellobiose as carbon feedstock (FIG. 9). For this, SGT 06-1™ was inoculated in 10 ml basic growth media in Durham test tubes containing increasing amounts of DE. The total gas generated after 10 hours incubation at 37° C. was measured as mm of gas bubble trapped within the inverted Durham tube. The gas production of SGT 06-1™ increased to about four fold from 7 mm gas without DE to about 28 mm gas with 1% DE in the test tube during the experiment. Maximum effect on gas production occurred with 1% DE present during the incubation. No gas production was observed in control test tubes filled with basic growth media and 1% DE in the absence of SGT 06-1™ (data not shown).

### Example 16

### Increased Hydrogen Production Rate in the Presence of Diatomaceous Earth with Different Carbohydrates as Feedstock

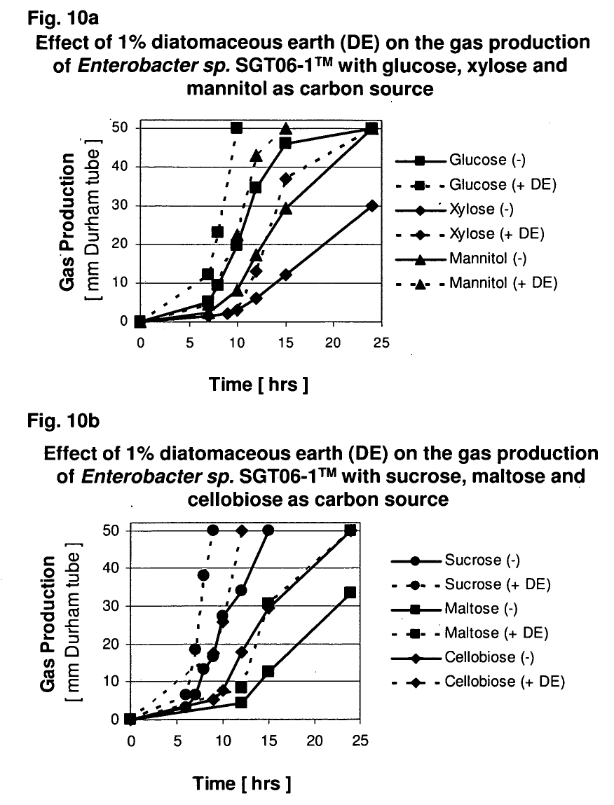

Next the effect of diatomaceous earth (DE) on the hydrogen production rate of SGT 06-1™ was tested in the presence of carbohydrates other than the cellulose-derived disaccharide cellobiose (FIGS. 10a and 10b). There was interest in whether SGT 06-1™ responds with a higher gas production rate in the presence of DE with other important renewable biomass-derived disaccharides, e.g. sucrose and maltose, as well as with the mono-sugars glucose, xylose and mannitol as carbon feedstock. As shown in FIG. 10a, SGT 06-1™ responds with much higher gas production with glucose as feedstock, and also when incubated in the presence of the hemicellulosics-contained monosugar xylose and with the alcoholic sugar mannitol as carbon source. As shown in Table B herein, the percent increase in hydrogen production rate of SGT 06-1™ was 231% for glucose, 254% for mannitol and 460% for xylose. In another set of experiments which results are shown in FIG. 10b, the effect of diatomaceous earth (DE) on the gas production of SGT 06-1™ was tested with the disaccharides sucrose (filled circles), maltose (filled squares) and cellobiose (filled diamonds). Solid lines represents absence of 1% DE and dashed lines represents presence of 1% DE. As shown in FIG. 10b in the presence of DE, SGT 06-1™ responds with much higher gas production for all three sugars. As shown in Table B, the percent increase in hydrogen production rate was with 306% highest for maltose, while SGT 06-1™ showed a percent increase of gas production rate of 283% in the presence of sucrose and 280% in the presence of cellobiose as carbon feedstock.

### Example 17

### Increased Hydrogen Production Rate of SGT06-1™ in the Presence of Diatomaceous Earth and Zeolite

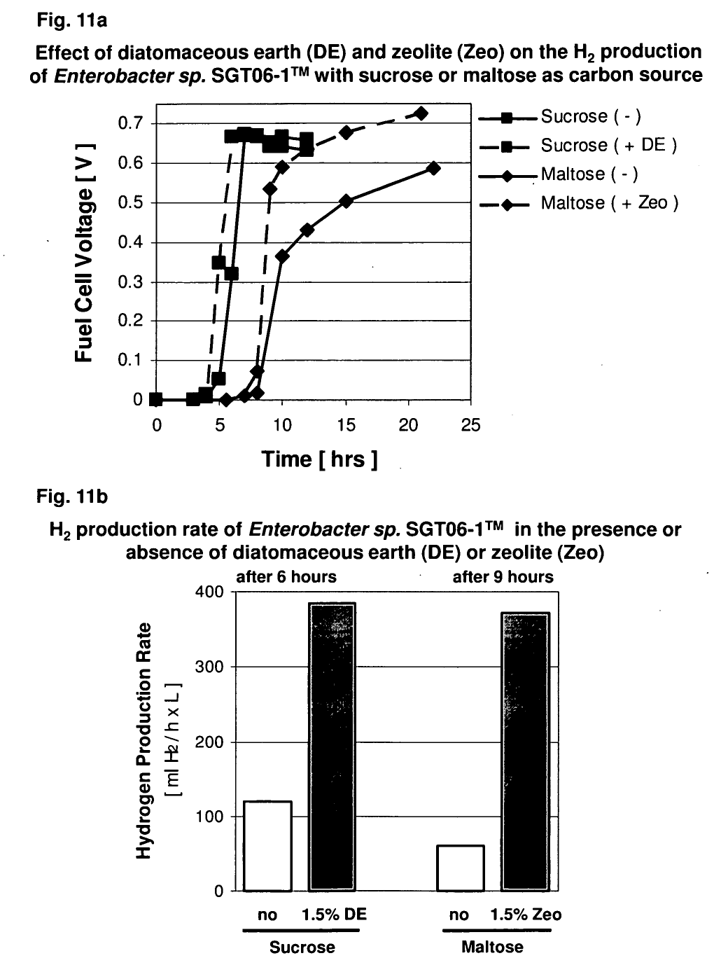

A further set of studies was conducted to show that diatomaceous earth (DE) and zeolite (Zeo) not only increased the total gas production rate but also lead to an increased hydrogen gas production rate of Enterobacter sp. SGT06-1. For this, SGT06-1™ was inoculated and incubated in the absence (solid lines) or presence (dashed lines) of 1.5% DE (filled squares) or 1.5% Zeo (filled diamonds) under conditions as further described in Example 14 and in the presence of 2.5% sucrose (filled squares) or 2.5% maltose (filled diamonds) as carbon source. Time-dependent generation of hydrogen gas (H₂) was continuously monitored with the help of an in-line coupled fuel cell system (FIG. 11a) and the hydrogen production rate of SGT06-1™ (FIG. 11b) for the different sugars was determined using an established calibration curve of the fuel cell system (not shown).

In the presence of DE and Zeo, SGT06-1™ responds with a clearly earlier generation of hydrogen and also shows significantly higher hydrogen production rates. With 2.5% sucrose as feedstock and after 5 hours incubation, SGT06-1™ shows a calculated hydrogen production rate of 120 ml H₂ per liter per hour in the absence of DE while a hydrogen production rate of 384 ml H₂ per liter per hour was observed in the presence of 1.5% DE in the fermentation vessel (FIG. 11b). With 2.5% maltose as feedstock and after 9 hours incubation, SGT06-1™ shows a calculated hydrogen production rate of 60 ml H₂ per liter per hour in the absence of zeolites and a hydrogen production rate of 374 ml H₂ per liter per hour in the presence of 1.5% zeolites in the fermentation vessel (FIG. 11b). Summarized, presence of the absorptive materials DE or Zeolite in the fermentation vessel lead to a 320% (DE; sucrose) and 620% (Zeo; maltose) increase in hydrogen production rate under the described batch incubation conditions.

### Example 18

### Effect of Diatomaceous Earth on the Gas Production Rate of Different Enterobacteria

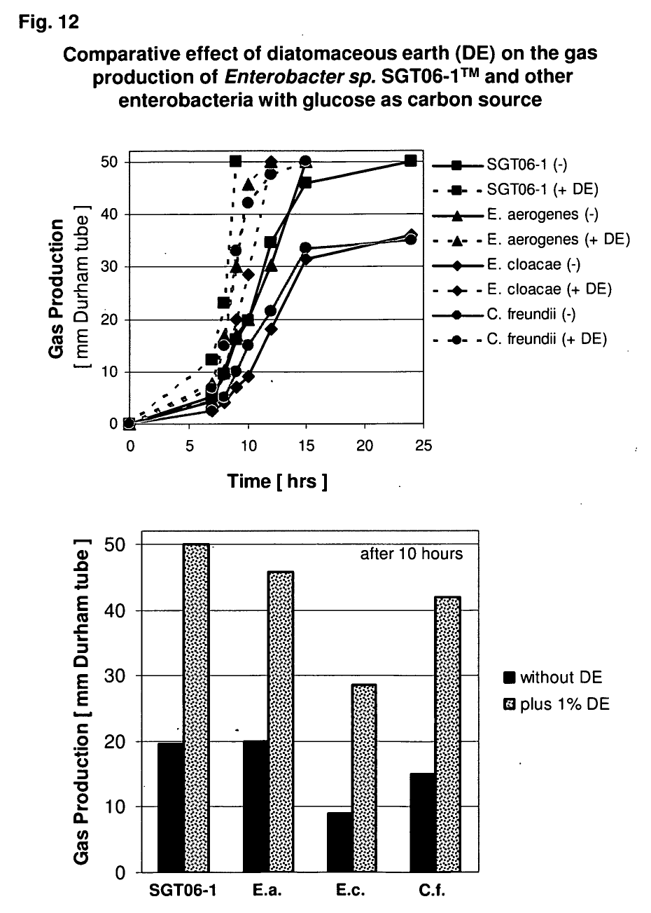

Another set of studies was conducted to show that the presence of diatomaceous earth (DE) during the disclosed incubation conditions not only increased the gas production rate of Enterobacter sp. SGT06-1™ but of other known gas producing bacteria as well. As shown in FIGS. 12a and 12b, the presence of 1% DE during the incubation of different bacteria in Durham tubes (dashed lines), lead to increased gas production from Enterobacter sp. SGT06-1™ (filled squares), Enterobacter aerogenes ATCC 13048 (E. aerogenes; filled triangles), Enterobacter cloacae ATCC 15361 (E. cloacae; filled diamonds), and Citrobacter freundii ATCC 13316 (C. freundii; filled circles). In the presence of DE, the gas production after 10 hours incubation with the different bacteria was about 250% higher with Enterobacter sp. SGT06-1™, about 235% higher with E. aerogenes, about 310% higher with E. cloacae and about 300% higher with C. freundii, when compared to test tubes containing these gas producing microbes in the absence of DE (dotted bars versus filled bars of FIG. 12b). Because DE under fermentation conditions leads to an increased gas production rate with SGT 06-1™, and other gas producing bacteria, it makes the disclosed process broadly usable for industrial scale generation of hydrogen energy using diverse microorganisms.

### Example 19

### Increased Gas Production Rate in the Presence of Other Absorptive Materials

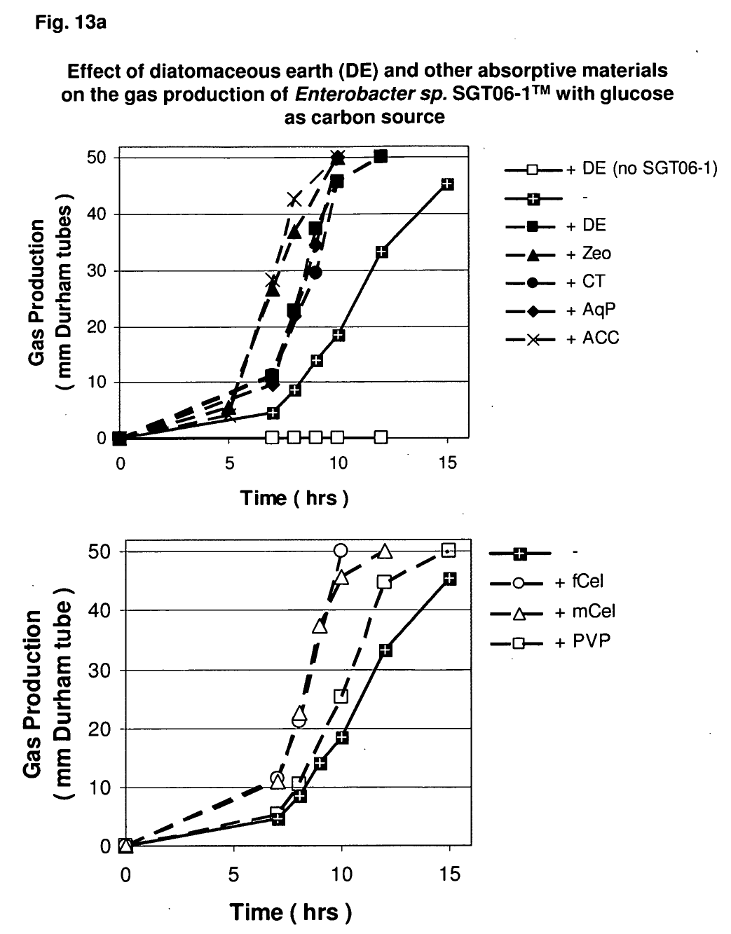

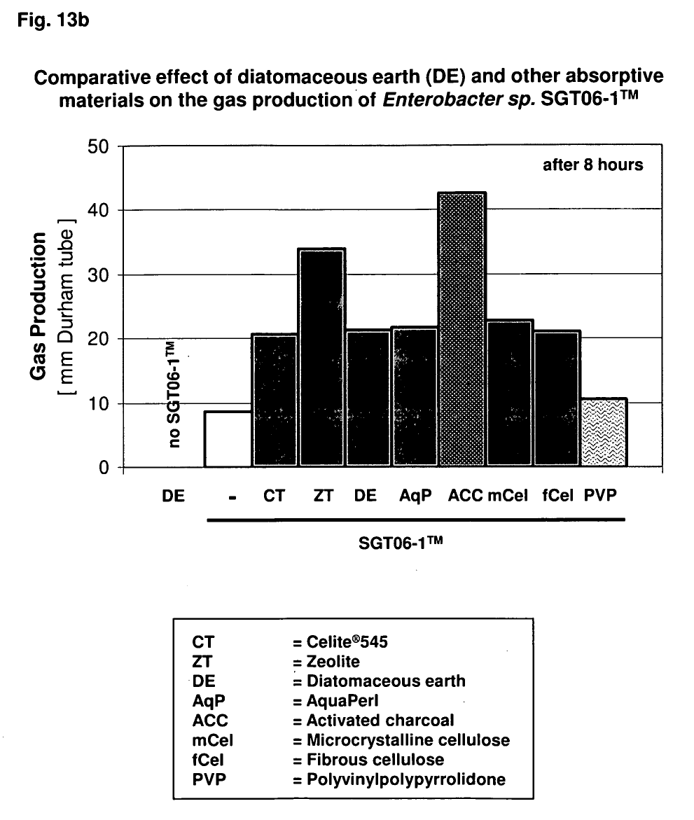

Another set of studies was conducted to show increased gas production rate of Enterobacter sp. SGT06-1™ not only with diatomaceous earth (DE) present during the incubation conditions (37° C., basic growth medium), but also when incubated with other known highly absorptive materials. As shown in FIG. 13a, SGT06-1™ shows an increased gas production not only in the presence of DE (filled squares; dashed line) during the incubations, but also in the presence of natural zeolite (Aquatic Gardens™) (Zeo; filled triangles; dashed line), the silicaceous material Celite®545 (Sigma) (CT; filled circles; dashed line); AquaPerl material (Harborlite Inc. Lompoc, Calif.) (AqP; filled diamonds; dashed line), activated charcoal (ACC; crosses; dashed line), microgranular cellulose powder (Sigma) (mCel; open triangles; dashed line), and of fibrous cellulose powder (Sigma) (fCel; open circles; dashed line). Interestingly, a much less pronounced increase in gas production was observed when SGT06-1™ was incubated in the presence of the absorptive synthetic polymer polyvinylpolypyrrolidone (Sigma) (PVP; open squares; dashed line) under identical incubation conditions. The results of experiments conducted with SGT06-1™ under identical incubation conditions but without absorptive materials in the reaction tubes are shown for comparison (filled squares with center cross; solid line).

Control experiments conducted with tubes containing DE but no SGT06-1™ bacteria did not show any gas production (open squares; solid line). The concentration of the absorptive materials under investigation was 1% and all incubations were conducted with 2.5% glucose as carbon source. Based on these experiments, it appears that the increased gas production effect is strongly connected with their (known) highly absorptive nature. Without being bound by theory, and offered to improve the understanding of the disclosure, the materials may contribute to increased hydrogen gas production by a mechanism involving absorbing (currently unknown) fermentative intermediate- and/or end products which would otherwise be detrimental or toxic to the gas generating enzyme system(s) of the bacteria.

### Example 20

### Increased Gas Production Rate in the Presence of Diatomaceous Earth with Processed Brewery Waste as Feedstock

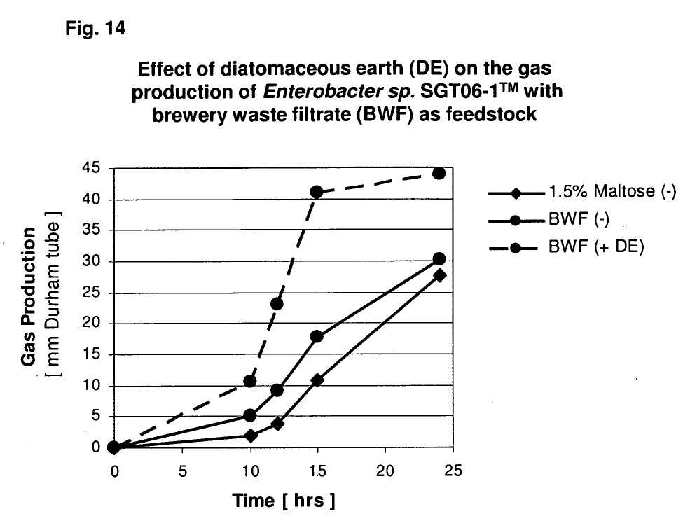

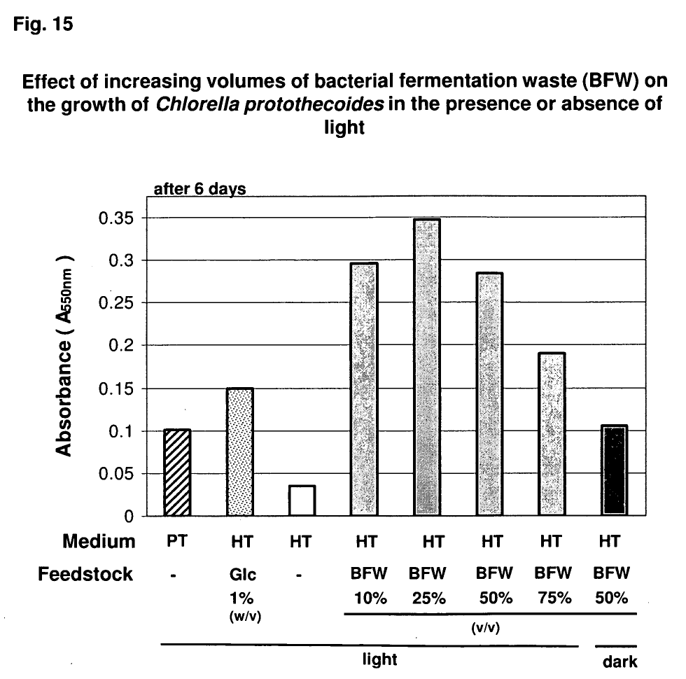

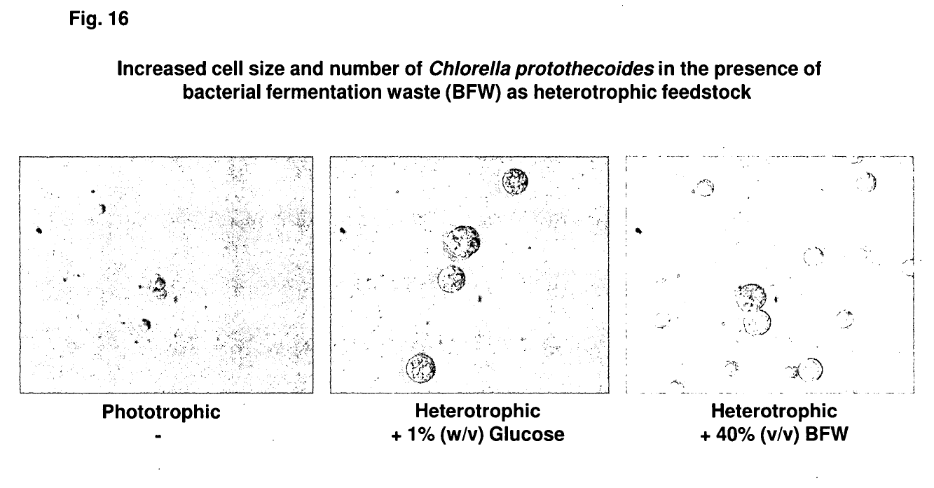

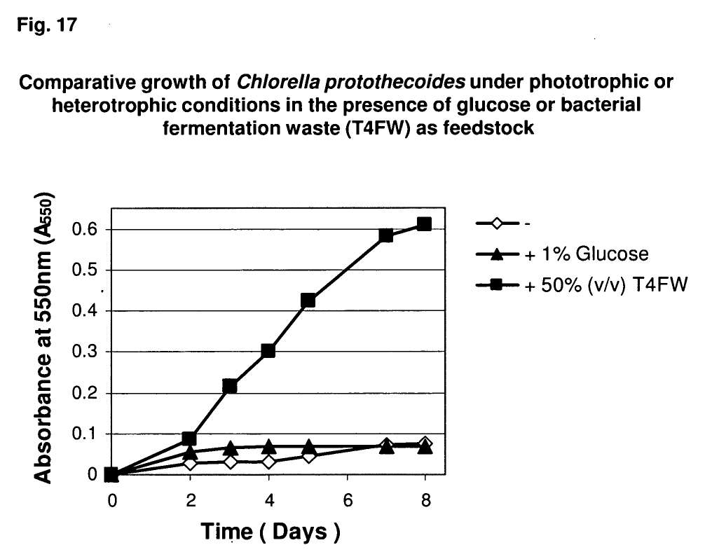

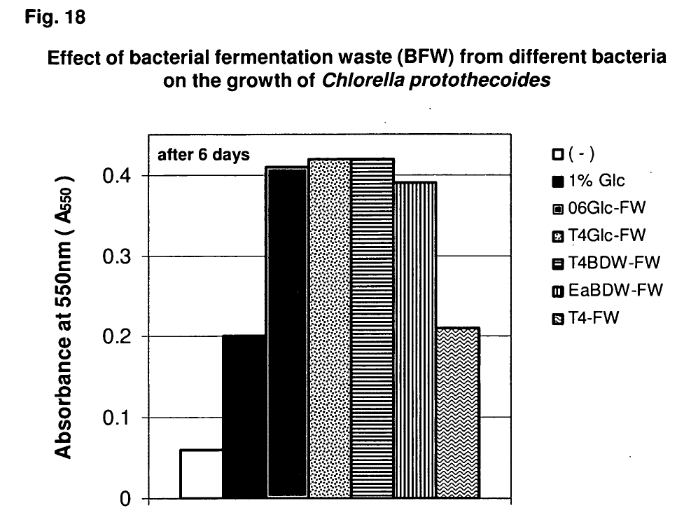

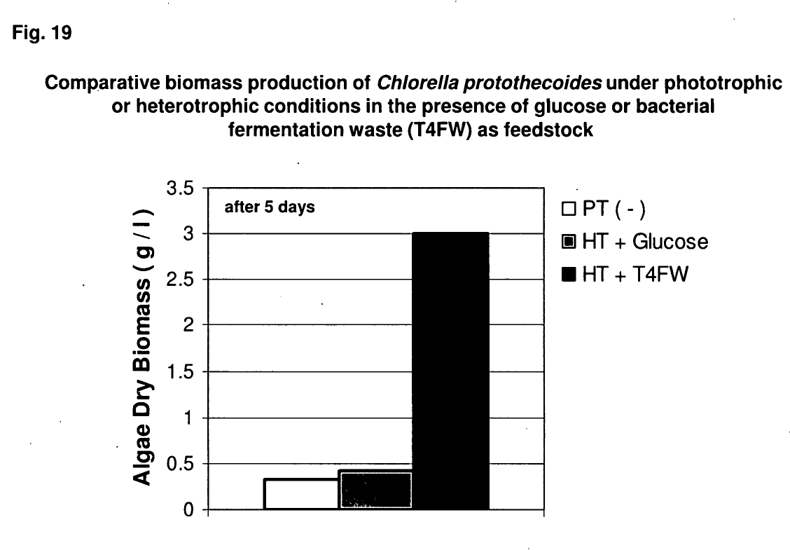

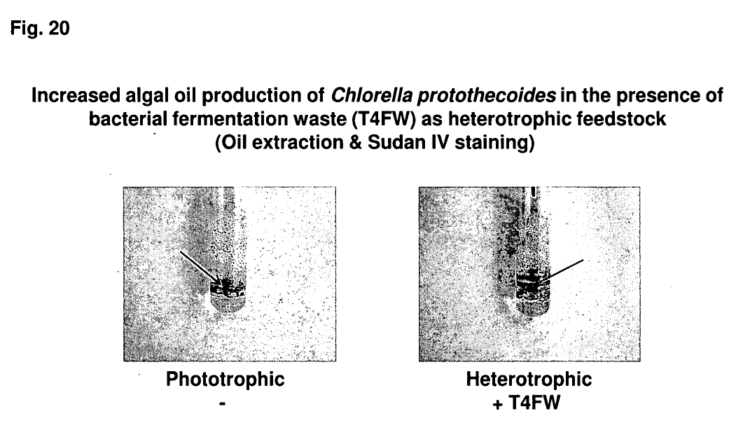

Another set of studies was conducted to show increased gas production rate of Enterobacter sp. SGT06-1™ in the presence of diatomaceous earth (DE) with processed brewery waste instead of chemically defined sugars, e.g. glucose or cellobiose, used in the previous studies as carbon feedstock. As shown in FIG. 14, SGT06-1™ was incubated in Durham test tubes at 37° C. containing basic growth medium and sterile brewery waste filtrate (BWF; filled circles) in the absence (solid line) and presence (dashed line) of 1% DE. Sterile brewery waste filtrate (BWF) was prepared using following procedure. Freshly collected and blended brewery waste (San Marcos Brewery, San Marcos, Calif.) was incubated at a concentration of 30% (w/v) in 20 mM potassium phosphate buffer, pH 7.0, supplemented with 1 mM CaCl₂ and 30 U/ml pancreatic α-amylase (Sigma) at 37° C. for 8 hours under constant stirring. Then the brewery waste slurry was pressed through a cheese cloth and further filtered from debris and fibrous materials using a series of Whatman quality filter materials each with different pore sizes. Finally, the filtrate was filtered using a 0.45 mm sterile filtration unit (Corning) before used in the fermentation experiments described above.

As shown in FIG. 14, DE increased the gas production rate of SGT06-1™ with brewery waste filtrate as feedstock about 260%. Also DE increased the gas production rate 400% compared to 1.5% maltose (solid diamonds) as carbon source.

### Example 21

### Microalga and Algal Growth

A strain of Chlorella protothecoides (Krueger UTEX strain #25) disclosed herein was provided by the Culture Collection of Algae at the University of Texas (Austin, Tex., U.S.A.). The algae was routinely grown and propagated in sterile Proteose Bristol growth medium (2.94 mM NaNO₃, 0.17 mM CaCl₂×2H₂O, 0.3 mM MgSO₄×7H₂O, 0.43 mM K₂HPO₄, 1.29 mM KH₂PO₄, 0.43 mM NaCl, 1 g peptone per liter, pH 6.8) as recommended by the supplier. The algae was routinely grown photoautotrophically and axenically in batch cultures at a temperature of 25° C. (+/−1.5° C.) under dim natural day light conditions. Continuous aeration was provided by bubbling micro-filtered air (0.22 μm pore size) at regular pressure using an air pump (Aquatic Gardens™ model 1500). For organoheterotrophic growth of C. protothecoides with processed bacterial fermentation wastes as carbon feedstock, a special cultivation medium was devised.

For heterotrophic cultivation of the microalgae in the presence of processed bacterial fermentation waste following heterotrophic (HT) cultivation medium was used: 0.3 g K₂HPO₄, 0.7 g KH₂PO₄, 0.3 g MgSO₄×7H₂O, 3 mg FeSO₄×7H₂O, 0.1 g glycine, 10 ml 100× Arnon's A solution (per liter). Arnon's A solution was prepared following a standard recipe known in the field of algal cultivation. For microalgal growth in the presence of aqueous bacterial fermentation wastes, 1 liter of double-concentrated HT cultivation medium was mixed with 1 liter of processed bacterial fermentation waste and the culture medium was inoculated with 10 ml of a 4-6 day old phototrophic microalgae culture. The algae were grown axenically and without stirring under batch cultures at a temperature of 25° C. (+/−1.5° C.) under dim natural day light conditions. Continuous aeration was provided by bubbling micro-filtered air (0.22 μm pore size) at regular pressure using an air pump (Aquatic Gardens™ model 1500).

### Example 22

### Processing of Aqueous Bacterial Fermentation Waste Stream

Processed bacterial fermentation waste suitable as heterotrophic, low cost microalgal feedstock was prepared as following. A suitable microorganism, such as Enterobacter sp. SGT-T4 or Enterobacter sp. SGT06-1, is grown for 24 hours in tryptone-yeast (TY) medium in the presence of a suitable feedstock, preferentially but exclusively bio-diesel refinery waste under conditions as described herein and in the presence or absence of a silicaceous material, such as, but not limited to, zeolite. After fermentation of the supplied feedstock, e.g. bio-diesel refinery waste, the growth medium is collected at defined time points, preferentially but not exclusively after 24 hours, and the included microorganisms were separated from the fermentation medium by centrifugation for 30 minutes at 3,300 rpm. The resulting supernatant was collected and sterile filtered using 0.2 μm filtration system (115 ml filter unit, SFCA, 0.2 microns, Nalgene).

### Example 23

### Microalgal Oil Extraction and Analysis

The microalgae is cultivated under aerated heterotrophic conditions in the presence of processed bacterial fermentation waste in heterotrophic (HT) cultivation medium (0.3 g K₂HPO₄, 0.7 g KH₂PO₄, 0.3 g MgSO₄×7H₂O, 3 mg FeSO₄×7H₂O, 0.1 g glycine, 10 ml 100× Arnon's A solution (per liter)). The microalgae is grown axenically and under batch cultures at a temperature of 25° C. (+/−1.5° C.) under dim natural day light conditions in cultivation flasks. Continuous aeration is provided by bubbling micro-filtered air (0.22 μm pore size) at regular pressure using an air pump (Aquatic Gardens™ model 1500). After 5-6 days, the microalgae culture is quantitatively harvested and concentrated by centrifugation for 15 minutes at 3,500 rpm. The pelleted microalgae are exposed to moderate temperatures in a heater oven until reaching complete dryness. The dried microalgal biomass is reconstituted in an equal volume of distilled water and milled in the presence of dry ice and 5-10% Celite (silicon dioxide) using a conventional porcelain mortar-pistil device. The microalgal oils in the thus prepared aqueous cell lysates are extracted by methods known in the arts of lipid/oil extraction, for example by repeatedly adding defined volumes of an organic solvent, such as chloroform, n-hexane, and/or mixtures thereof, to the microalgal lysates. The presence of oils in the subsequently evaporated organic solvent was established by adding defined amounts of the known oil indicator dye Sudan IV to the extractions.

### BIBLIOGRAPHY

### U.S. Patents and Published Applications

| | | | |
|---|---|---|---|
| 2,429,589 | October, 1947 | Wiley | 435/167. |
| 3,383,309 | May, 1968 | Chandler | 48/197. |
| 3,711,392 | January, 1973 | Metzger | 435/167. |
| 3,764,475 | October, 1973 | Mandels et al. | 435/209. |
| 4,480,035 | October, 1984 | Roychowdhury | 435/168. |
| 5,350,692 | September, 1994 | Taguchi et al. | 435/252.7 |
| 6,887,692 | December, 2002 | Paterek JR | 438/168. |
| 6,860,996 | March 2005 | Noike et al. | 210/603. |
| 6,942,998 | September, 2005 | Ooteghem | 435/168. |
| 5,213,690 | MAY 25, 1993 | WOLLRICH GARY W. | |
| 5,570,973 | NOV. 5, 1996 | HUNT SETH C. | |
| 5,603,836 | FEB. 18, 1997 | WOLLRICH GARY W. | |
| 20040216367 | NOV. 4, 2004 | KLEIN ARNOLD G. | |
| 5,792,631 | Aug. 11, 1998 | Running | |

Microbial process for the production of ascorbic acid
using Chlorella protothecoides

| | | |
|---|---|---|
| 7,063,957 | Jun. 20, 2006 | Chen F. |

Methods for production of astaxanthin from green microalgae
Chlorella in dark-heterotrophic cultures

| | | |
|---|---|---|
| 20030152587 | Aug. 14, 2003 | Kralovec J. A. |

Methods for making and using a Chlorella extract having
immunomodulatory properties

| | | |
|---|---|---|
| 22080096267 | Apr. 14, 2008 | Howard E. E., et al. |

Systems and methods for large scale production and
harvesting of oil-rich algae

| | | |
|---|---|---|
| 20070202582 | Aug. 30, 2007 | Bush R. A. et al. |

Process for production of ethanol from algae

| | | |
|---|---|---|
| 20070048848 | Mar. 1, 2007 | Sears J. T. |

Method, apparatus and system for biodiesel production from algae

### Other References

“List of UTEX Cultures”, Culture Collection of Algae, Department of Botany, University of Texas at Austin, pp. 23-24, (1993).
Ahmad et al., “Growth and Photosynthesis of Chlorella protothecoides Under Different Nutritional Conditions”, J. Phycology, 25, Supp. 2, (1989) (Abstract only).
Albertano et al., “Chlorella protothecoides Kruger var. acidicola, a new variety from very low pH environments”, Arch. Hydrobiol. Suppl. 67(4): 401-408 (1984).
Casselton et al., “Ammonium Assimilation Prototheca zopfii Kruger”, British Phycological Society 17(2): 230-231 (1982).
Kerfin et al., “Physiological and Biochemical Contributions to the Taxonomy of the Genus Chlorella”, Arch. Microbiol. 116: 97-103 (1978).
Vinayakumar M. & Kessler E. “Physiological and biochemical contributions to the taxonomy of the genus Chlorella. X. Products of glucose fermentation.” Arch. Microbiol. 103(1): 13-19 (1975).
Kessler, “Physiological and Biochemical Contributions to the Taxonomy of the Genus Prototheca, III. Utilization of Organic Carbon and Nitrogen Compounds”, Arch. Microbiol. 132: 103-106 (1982).
Kessler, “Comparative Physiology, Biochemistry, and the Taxonomy of Chlorella (Chlorophyceae)”, Plant Syst. Evol. 125: 129-138 (1976).
Kessler, “Physiologische and Biochemische Beitrage zur Taxonomie der Gattung Chlorella, IX. Salzresistenz als Taxonomisches Merkmal”, Arch. Microbiol. 100: 51-56 (1974).
Chisti Y. “Biodiesel from microalgae.” Biotechnol. Adv. 25(3): 294-306 (2007).
Chisti Y. “Biodiesel from microalgae beats bioethanol.” Trends Biotechnol. 26(3): 126-131 (2008).
Papazi A., et al. “Bioenergetic changes in the microalagal photosynthetic apparatus by extremely high CO2 concentrations induce an intense biomass production.” Physiol. Plant. 132(3): 338-349 (2008).
Miao X., et al. “Biodiesel production from heterotrophic microalgal oil.” Bioresource Technol. 97: 841-846 (2006).
Xu H., et al. “High quality biodiesel production from a microalga Chlorella protothecoides by heterotrophic growth in fermenters.” J. Biotechnol. 126(4): 499-507 (2006).
Ito T. et al., “Hydrogen and ethanol production from glycerol-containing wastes discharged after biodiesel manufacturing process” J. Biosci. Bioeng. 100(3):260-265 (2005).
Lee K.-S. et al., “H₂ production with anaerobic sludge using activated-carbon supported packed-bed bioreactors” Biotechnol. Lett. 25:133-138 (2003).
Thompson L. J. et al., “Biohydrogen production by Enterobacter cloacae and Citrobacter freundii in carrier induced granules” Biotechnol. Lett. 30:271-274 (2008).
Yokoi, H. et al., “H₂ production from starch by a mixed culture of Clostridium butyricum and enterobacter aerogenes” Biotechnol. Lett. 20(2):143-147 (1998).

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of increasing microbial hydrogen gas production, the method comprising the step of incubating a hydrogen producing microorganism, in the presence of absorptive material.

2. The method of claim 1, wherein
the rate and the amount of hydrogen gas production is increased;
the hydrogen producing microorganism is Enterobacter sp. SGT06-1™, Enterobacter aerogenes, Enterobacter cloacae, Citrobacter freundii, Clostridia sp., or Thermotogae sp.;
the absorptive material is diatomaceous earth (SiO₂), Celite®545, silica (SiO₂), silicates (SiO₄), mineral zeolites (aluminosilicates, olivine, feldspars, nepheline, vermiculite, or epidote), crystalline silicates, amorphous silicates, metal oxides, activated carbon (charcoal), cellulose, microcrystalline cellulose, crystalline cellulose, granular cellulose, or fibrous cellulose, and combinations and derivatives thereof;
the absorptive material is crude, semi-purified or purified silica (SiO₂) or silicates (SiO₄);
the silicate is a ring-silicate, a chain silicate, a sheet silicate, or a framework silicate;
the silica and silicates are in milli-, micro- or nanogranular solid or semi-solid form;
the absorptive material is crude, semi-purified or purified activated carbon (charcoal);
the activated carbon (charcoal) is in milli-, micro- or nanogranular solid or semi-solid form;
the absorptive material is a crude, semi-purified or purified metal oxide, wherein the metal oxide is titanium oxide (TiO₂), iron oxide (FeO, Fe₂O₃, Fe₃O₄), ilmenite (FeTiO₃), titanite (CaTiSiO₅), tin oxide (SnO or SnO₂), cerium oxide (CeO₂ or Ce₂O₃), zircon (ZrSiO₄), or aluminum oxide (Al₂O₃);
the metal oxides are in milli-, micro- or nanogranular solid or semi-solid form;
the absorptive material is crude, semi-purified or purified cellulose or derivatives thereof, optionally wherein one of the cellulose derivatives is carboxymethyl-cellulose (CMC) or the cellulose and derivatives thereof, are in milli-, micro- or nanogranular solid or semi-solid form; or
the absorptive material is integrated into a vessel in form of thin films, optionally wherein the thin film is a silica, metal oxide, cellulose or cellulose derivative thin film or the thin film is coated to a glass or ceramic surface.

3. The method of claim 1, further comprising incubating the hydrogen producing microorganism in diverse purified or crude feedstock,
wherein the feedstock is optionally arabinose, glucose, cellobiose, maltose, mannitol, rhamnose, sucrose, or xylose, or a combination thereof.

4. A method of collecting extraction and fermentation end- or by-products of a microorganism, comprising the step of incubating the microorganism in the presence of absorptive material.

5. The method of claim 4, wherein
the microorganism is a hydrogen producing microorganism;
the hydrogen producing microorganism is Enterobacter sp. SGT06-1™, Enterobacter aerogenes, Enterobacter cloacae, Citrobacter freundii, Clostridia sp., or Thermotogae sp.; and the absorptive material is diatomaceous earth (SiO₂), Celite®545, silica (SiO₂), silicates (SiO₄), mineral zeolites (aluminosilicates, olivine, feldspars, nepheline, vermiculite, or epidote), crystalline silicates, amorphous silicates, activated carbon (charcoal), metal oxides, cellulose, microcrystalline cellulose, crystalline cellulose, granular cellulose, or fibrous cellulose, and combinations and derivatives thereof; or
the fermentation end- or by-products are hydrogen gas (H₂), acetate, lactate, succinate, formate, butanediol, or butanol.

6. A method of culturing a microorganism, comprising the step of incubating the microorganism in the presence of absorptive material.

7. The method of claim 6, wherein
the microorganism is a hydrogen producing microorganism; or
the hydrogen producing microorganism is Enterobacter sp. SGT06-1™, Enterobacter aerogenes, Enterobacter cloacae, Citrobacter freundii, Clostridia sp., or Thermotogae sp.; and the absorptive material is diatomaceous earth (SiO₂), Celite®545, silica (SiO₂), silicates (SiO₄), mineral zeolites (aluminosilicates, olivine, feldspars, nepheline, vermiculite, or epidote), crystalline silicates, amorphous silicates, activated carbon (charcoal), metal oxides, cellulose, microcrystalline cellulose, crystalline cellulose, granular cellulose, or fibrous cellulose, and combinations and derivatives thereof.

8. A method of extracting fermentative microbial by- or end-products, the method comprising:
a) incubating a microorganism in the presence of absorptive material and growth media,
b) separating the absorptive material from the growth media, and
c) extracting the fermentative microbial by- or end-products from the absorptive material.

9. The method of claim 8, wherein
the microorganism is a hydrogen producing microorganism;
the hydrogen producing microorganism is Enterobacter sp. SGT06-1™, Enterobacter aerogenes, Enterobacter cloacae, Citrobacter freundii, Clostridia sp., or Thermotogae sp.; and the absorptive material is diatomaceous earth (SiO₂), Celite®545, silica (SiO₂), silicates (SiO₄), mineral zeolites (aluminosilicates, olivine, feldspars, nepheline, vermiculite, or epidote), crystalline silicates, amorphous silicates, activated carbon (charcoal), metal oxides, cellulose, microcrystalline cellulose, crystalline cellulose, granular cellulose, or fibrous cellulose, and combinations and derivatives thereof; or
the fermentative microbial by- or end-products are hydrogen gas (H₂), acetate, lactate, succinate, formate, butanediol, or butanol.

10. A method of producing high amounts of microalgal biomass and oil comprising cultivating a green microalga belonging to the Chlorella species under ambient light conditions or in the dark in a cultivation medium containing a defined volume of processed bacterial fermentation waste, or a waste produced by a method of claim 1, and recovering microalgal oil therefrom.

11. The method of claim 10, wherein
the cultivation medium contains 25% (v/v), 30% (v/v), 35% (v/v), 40% (v/v), 45% (v/v), 50% (v/v), 55% (v/v), 60% (v/v), 65% (v/v), 70% (v/v), or 75% (v/v) of processed bacterial fermentation waste;
the green microalga Chlorella used in the culture is Chlorella protothecoides UTEX strain #25;
the processed bacterial fermentation waste derives from a fermentation reaction using a bacterium belonging to the enterobacteriaceae family, optionally, the enterobacteria is the enterobacterium Enterobacter sp. SGT-T4 or the enterobacterium Enterobacter sp. SGT06-1;
the fermentation reaction contains the enterobacterium Enterobacter sp. SGT-T4 and defined amounts of bio-diesel refinery waste;
the fermentation reaction contains the enterobacterium Enterobacter sp. SGT-T4 and defined amounts of cellulosics- or starch-derived carbohydrates, preferentially but not exclusively glucose, cellobiose, cellotriose, cellotetraose, and/or maltose;
the fermentation reaction contains the enterobacterium Enterobacter sp. SGT-T4 and defined amounts of hemicellulosics-derived carbohydrates, preferentially but not exclusively xylose, arabinose and/or galactose;
the fermentation reaction contains the enterobacterium Enterobacter sp. SGT06-1 and defined amounts of cellulosics-derived carbohydrates, preferentially but not exclusively glucose, cellobiose, cellotriose, and/or cellotetraose;
the fermentation reaction contains the enterobacterium Enterobacter sp. SGT06-1 and defined amounts of hemicellulosics-derived carbohydrates, preferentially but not exclusively xylose, arabinose and/or galactose;
the fermentation reaction contains a bacterium belonging to the enterobacteriaceae family and defined volumes of animal or human urine;
the microalgal culture medium is continuously aerated with a pure gas or a gas mixture.

12. The method of claim 10, wherein the fermentation reaction contains defined amounts of a silicaceous material in macro-, micro- or nano-granular form, optionally wherein
the silicaceous material is diatomaceous earth, a natural zeolite or a synthetic zeolite;
the silicaceous material is added to the fermentation reaction at a concentration of about 1.5%, or about 2%, or about 2.5%, or about 3%, or about 3.5%, or about 4%;

13. The method of claim 10, wherein the fermentation reaction contains defined amounts of one or more alga growth and oil production promoting compounds, optionally wherein
the alga growth and oil production promoting compound is biotin, or panthothenic acid, or folic acid, or riboflavin, or nicotinic acid, and/or combinations thereof.

14. The method of claim 10, wherein the microalgal culture medium is continuously aerated with a pure gas or a gas mixture and
the pure gas is carbon dioxide, or
the gas mixture contains carbon dioxide, oxygen, and nitrogen in defined concentrations.
